(19) United States
(12) Patent Application Publication
(10) Pub. No.: US 2020/0385406 A1
(43) Pub. Date: Dec. 10, 2020

(54) SUBSTITUTED HETEROCYCLIC-PYRIDINONES AS HIV-1 NEF-HCK INHIBITORS

(71) Applicants: Southern Research Institute, Birmingham, AL (US); University of Pittsburgh-Of The Commonwealth System of Higher Education, Pittsburgh, PA (US)

(72) Inventors: Corinne E. Augelli-Szafran, Homewood, AL (US); Omar Moulkha-Chafiq, Birmomgjam, AL (US); Thomas E. Smithgall, Wexford, PA (US); Lori A. Emert-Sedlak, CranberryTwp., PA (US); Habin Shi, Pitsburgh, PA (US); Samuel Tanner, Hoover, AL (US); Vibha Pathak, Hoover, AL (US)

(21) Appl. No.: 16/769,941

(22) PCT Filed: Dec. 20, 2018
(86) PCT No.: PCT/US2018/066920

§ 371 Date: Jun. 4, 2020

Related U.S. Application Data
(60) Provisional application No. 62/609,991, filed on Dec. 22, 2017.

(51) Int. Cl. C07D 513/04

(52) U.S. Cl. CPC C07D 513/04

(57) ABSTRACT

Disclosed are heterocyclic-pyridinone analogs that are capable of inhibiting Nef-Hck and methods of treating viral infections such as, for example, HIV-1. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 62/609,991, filed on Dec. 22, 2017, the contents of which are incorporated herein by reference in their entirety.

### STATEMENT REGARDING FEDERALLY SPONSORED RESEARCH

This invention was made with government support under grant numbers HHSN272201400010I and HHSN27200022 awarded by the Department of Health and Human Services. The government has certain rights in the invention.

### BACKGROUND

HIV-1 nef encodes a small myristoylated protein required for optimal viral replication and AIDS pathogenesis (Fackler and Bauer (2002) Immunity 16: 493-497; Geyer et al. (2001) EMBO Rep. 2: 580-585). Deletion of nef from the HIV-related simian immunodeficiency virus prevents AIDS-like disease progression in rhesus macaques (Kestler et al. (1991) Cell 65: 651-662). In addition, expression of the nefgene alone is sufficient to induce an AIDS-like syndrome in transgenic mice very similar to that observed upon expression of the complete HIV-1 provirus (Hanna et al. (1998) Cell 95: 163-175; Hanna et al. (1998) J Virol. 72: 121-132). In humans, nef sequence variability and function correlate with HIV disease progression over the course of infection (Carl et al. (2001) J Virol. 75: 3657-3665; Kirchhoff et al. (1999) J. Virol. 73: 5497-5508). Indeed, long-term non-progressive HIV infection has been associated with nef-defective strains of HIV in some cases (Deacon et al. (1995) Science 270: 988-991; Kirchhoff et al. (1995) N. Engl. J. Med. 332: 228-232; Zou et al. (2012) Retrovirology 9:44). These and other studies identify the HIV-1 Nef accessory protein as a key molecular determinant of AIDS.

Nef lacks any known intrinsic enzymatic or biochemical function and instead exploits multiple host cell signaling pathways to optimize conditions for viral replication and AIDS progression (Malim and Emerman (2008) Cell Host Microbe 3: 388-398; Foster and Garcia (2008) Retrovirology 5: 84). Growing evidence identifies the Src family kinases (SFKs) as key molecular targets for Nef (Saksela, K. (2011) Curr. HIVRes. 9: 531-542). One important example is Hck, a Src family member expressed in macrophages that binds strongly to Nef via an SH3-mediated interaction (Lee et al. (1995) EMBO J. 14: 5006-5015; Arold et al. (1998) Biochemistry 37: 14683-14691). Nef binding leads to constitutive Hck activation (Moarefi et al. (1997) Nature 385: 650-653; Briggs et al. (1997) J. Biol. Chem. 272: 17899-17902), which may be important for macrophage survival (Briggs et al. (2001) J. Biol. CHem. 272: 17899-17902) and productive infection by M-tropic HIV (Komuro et al. (2003) J. Exp. Med. 198: 443-453). In addition, activation of Hck, Lyn or c-Src is a critical first step in the downregulation of cell-surface MHC-I by Nef, which enables immune escape of HIV-infected cells (Dikeakos et al. (2010) Mol. Biol. Cell 21: 3279-3292; Atkins et al. (2008) J Biol. Chem. 283: 11772-11784; Hung et al. (2007) Cell Host Microbe 1: 121-133). Transgenic mice expressing a Nef mutant lacking a highly conserved PxxPxR motif essential for activation of Hck and other SFKs showed no evidence of AIDS-like disease (Hanna et al. (2001) J. Virol. 75: 9378-9392). When the Nef-transgenic mice were crossed into a hck-null background, appearance of the AIDS-like phenotype was delayed with reduced mortality (Hanna et al. (2001) J. Virol. 75: 9378-9392). These observations support an essential role for Nef interactions with Hck and other SFKs in multiple aspects of AIDS pathogenesis.

Despite the established link between Nef-Hck inhibition and HIV-1, potent small molecules inhibitors of Nef-Hck have continued to remain elusive. Accordingly, there remains a need for small molecule inhibitors of Nef-Hck and methods of making and using same.

### SUMMARY

In accordance with the purpose(s) of the invention, as embodied and broadly described herein, the invention, in one aspect, relates to heterocyclic-pyridinone compounds useful in the treatment of disorders associated with a dysregulation of Nef-Hck including, but not limited to, viral infections, in particular, HIV-1.

Disclosed are compounds having a structure represented by a formula:

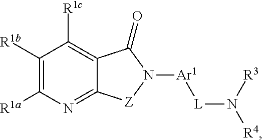

wherein Z is selected from O and S; wherein L is selected from C═O and SO₂; wherein each of R^{1a}, R^{1b}, and R^{1c} is independently selected from hydrogen, —NH₂, C1-C4 alkyl, C1-C4 haloalkyl, —CO₂H, —CO₂(C1-C4 alkyl), C1-C4 alkylamino, (C1-C4)(C1-C4) dialkylamino, and Cy¹; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; wherein Ar¹ is selected from 6-membered monocyclic aryl and pyridinyl, and is substituted with 0, 1, 2, 3, or 4 R² groups; wherein each occurrence of R², when present, is independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl); or wherein any two adjacent R² groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl); wherein R³ is hydrogen or C1-C4 alkyl; wherein R⁴ is selected from C1-C8 alkyl, Cy², and (C1-C4)Cy²; wherein Cy², when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; or wherein each of R³ and R⁴ are covalently bonded together and, together with the intermediate atoms, comprise a 3- to 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino, or a pharmaceutically acceptable salt thereof.

Also disclosed are pharmaceutical compositions comprising a therapeutically effective amount of at least one disclosed compound and a pharmaceutically acceptable carrier.

Also disclosed are methods for the treatment of a viral infection in a subject, the method comprising the step of administering to the subject an effective amount of at least one disclosed compound.

Also disclosed are kits kit comprising at least one disclosed compound and one or more of: (a) at least one antiviral agent; (b) a instructions for administering the at least one compound in connection with treating a viral infection; (c) instructions for administering the at least one compound in connection with reducing the risk of viral infection; and (d) instructions for treating a viral infection.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

Listed below are definitions of various terms used to describe this invention. These definitions apply to the terms as they are used throughout this specification, unless otherwise limited in specific instances, either individually or as part of a larger group.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a viral infection. The term “patient” includes human and veterinary subjects. In some aspects of the disclosed methods, the subject has been diagnosed with a need for treatment of one or more viral infections prior to the administering step. In various aspects, the one or more viral infections is HIV-1.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a viral infection prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “treating” refers to relieving the disease, disorder, or condition, i.e., causing regression of the disease, disorder, and/or condition. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein.

The term “comprising” (and its grammatical variations) as used herein is used in the inclusive sense of “having” or “including” and not in the exclusive sense of “consisting of.”

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

“Pharmaceutically acceptable salts” refer to derivatives of the disclosed compounds wherein the parent compound is modified by making acid or base salts thereof. The compounds of this disclosure form acid addition salts with a wide variety of organic and inorganic acids and include the physiologically acceptable salts which are often used in pharmaceutical chemistry. Such salts are also part of this disclosure. Typical inorganic acids used to form such salts include hydrochloric, hydrobromic, hydroiodic, nitric, sulfuric, phosphoric, hypophosphoric acid, and the like. Salts derived from organic acids, such as aliphatic mono- and dicarboxylic acids, phenyl substituted alkanoic acids, hydroxyalkanoic and hydroxyalkandioic acids, aromatic acids, aliphatic and aromatic sulfonic acids may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, O-hydroxybutyrate, butyne-1,4-dioate, hexyne-1,4-dioate, caprate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

It is understood that the compounds of the present disclosure relate to all optical isomers and stereo-isomers at the various possible atoms of the molecule, unless specified otherwise. Compounds may be separated or prepared as their pure enantiomers or diasteriomers by crystallization, chromatography or synthesis.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

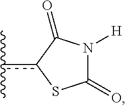

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. “Solvates” refers to the compound formed by the interaction of a solvent and a solute and includes hydrates. Solvates are usually crystalline solid adducts containing solvent molecules within the crystal structure, in either stoichiometric or nonstoichiometric proportions. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

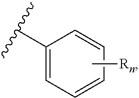

which is understood to be equivalent to a formula:

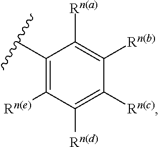

wherein n is typically an integer. That is, Rⁿ is understood to represent five independent substituents, R^{n(a)}, R^{n(b)}, R^{n(c)}, R^{n(d)}, R^{n(e)}. In each such case, each of the five R can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further aspects, a structure of a compound can be represented by a formula:

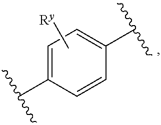

wherein R^{y} represents, for example, 0-2 independent substituents selected from A¹, A², and A³, which is understood to be equivalent to the groups of formulae:
wherein R^{y} represents 0 independent substituents

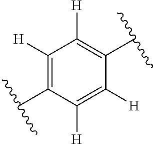

wherein R^{y} represents 1 independent substituent

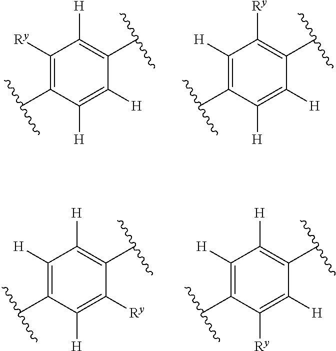

wherein R^{y} represents 2 independent substituents

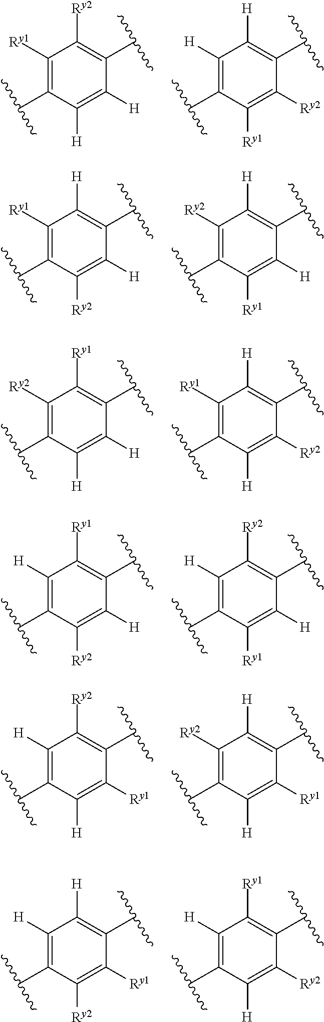

Again, by “independent substituents,” it is meant that each R substituent can be independently defined. For example, if in one instance R^{y1} is A¹, then R^{y2} is not necessarily A¹ in that instance.

In some further aspects, a structure of a compound can be represented by a formula,

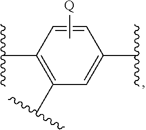

wherein, for example, Q comprises three substituents independently selected from hydrogen and A, which is understood to be equivalent to a formula:

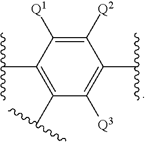

Again, by “independent substituents,” it is meant that each Q substituent is independently defined as hydrogen or A, which is understood to be equivalent to the groups of formulae:
wherein Q comprises three substituents independently selected from H and A

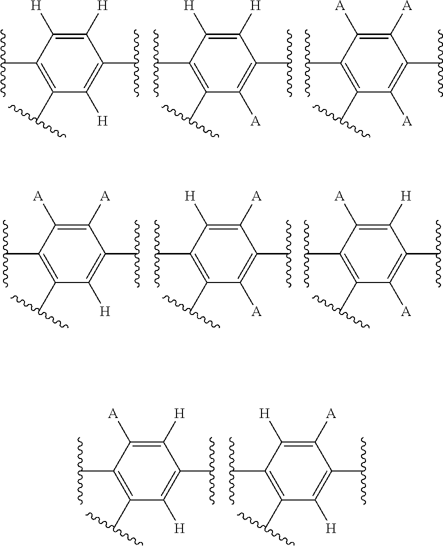

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Nef-HCK Inhibitors

In one aspect, the invention relates to compounds, or pharmaceutically acceptable derivatives thereof, useful as Nef-HCK inhibitors. In general, it is contemplated that each disclosed compound or derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

In one aspect, the compounds of the invention are useful in the treatment of viral infections. In a further aspect, the compounds are useful in the treatment of diseases associated with a viral infection. In a still further aspect, the compounds are useful in the treatment of HIV.

### 1. Structure

In one aspect, disclosed are compounds having a structure represented by a formula:

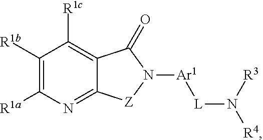

wherein Z is selected from O and S; wherein L is selected from C═O and SO₂; wherein each of R^{1a}, R^{1b}, and R^{1c} is independently selected from hydrogen, —NH₂, C1-C4 alkyl, C1-C4 haloalkyl, —CO₂H, —CO₂(C1-C4 alkyl), C1-C4 alkylamino, (C1-C4)(C1-C4) dialkylamino, and Cy¹; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; wherein Ar¹ is selected from 6-membered monocyclic aryl and pyridinyl, and is substituted with 0, 1, 2, 3, or 4 R² groups; wherein each occurrence of R², when present, is independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl); or wherein any two adjacent R² groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂(C1-C4 alkyl)(C1-C4 alkyl), —SO₂NH₂, —SO₂NH(C1-C4 alkyl), and —SO₂NH(C1-C4 alkyl)(C1-C4 alkyl); wherein R³ is hydrogen or C1-C4 alkyl; wherein R⁴ is selected from C1-C8 alkyl, Cy², and (C1-C4)Cy²; wherein Cy², when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; or wherein each of R³ and R⁴ are covalently bonded together and, together with the intermediate atoms, comprise a 3- to 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino, or a pharmaceutically acceptable salt thereof.

In a further aspect, the compound has a structure represented by a formula selected from:

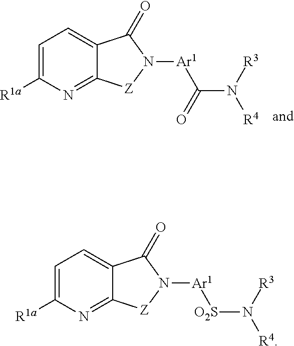

In a further aspect, the compound has a structure represented by a formula:

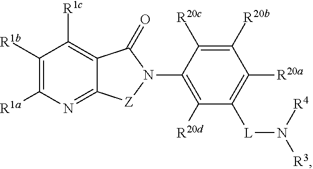

wherein each of R^{20a}, R^{20b}, R^{20c}, and R^{20d} is independently selected from hydrogen, halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂(C1-C4 alkyl)(C1-C4 alkyl), —SO₂NH₂, —SO₂NH(C1-C4 alkyl), and —SO₂NH(C1-C4 alkyl)(C1-C4 alkyl); or wherein any two adjacent R^{20a}, R^{20b}, R^{20c}, and R^{20d} groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂(C1-C4 alkyl)(C1-C4 alkyl), —SO₂NH₂, —SO₂NH(C1-C4 alkyl), and —SO₂NH(C1-C4 alkyl)(C1-C4 alkyl).

In a further aspect, the compound has a structure represented by a formula selected from:

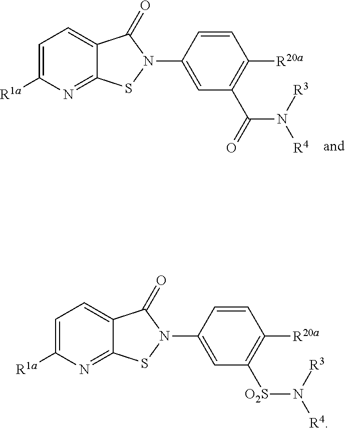

In a further aspect, the compound has a structure represented by a formula:

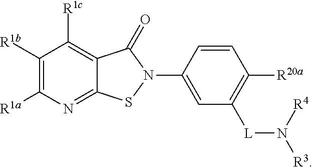

In a further aspect, the compound has a structure represented by a formula:

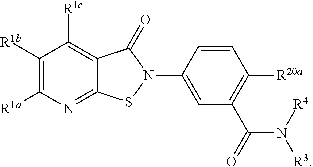

In a further aspect, the compound has a structure represented by a formula selected from:

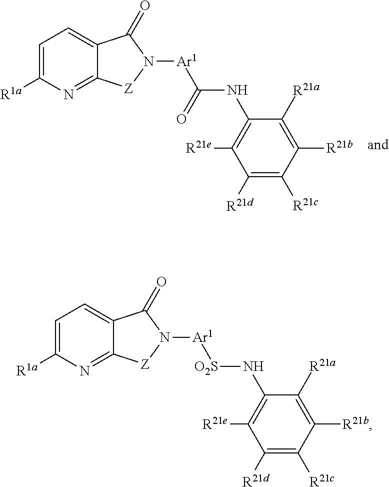

wherein each of R^{21a}, R^{21b}, R^{21c}, and R^{21d} is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino.

In a further aspect, the compound has a structure represented by a formula:

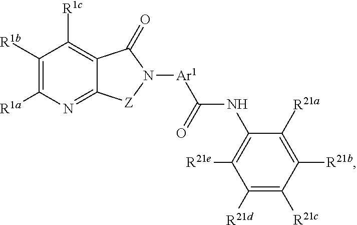

wherein each of R^{21a}, R^{21b}, R^{21c}, and R^{21d} is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino.

In a further aspect, the compound has a structure represented by a formula:

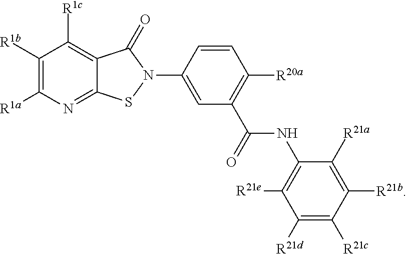

In a further aspect, the compound has a structure represented by a formula selected from:

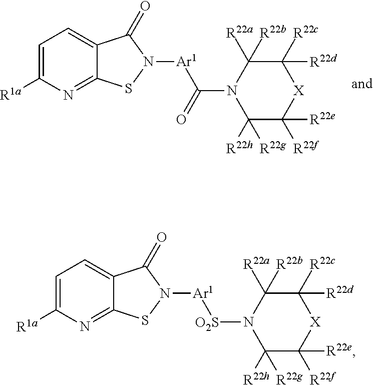

wherein X is selected from N, O, and CR^{30a}R^{30b}; wherein each of R^{30a} and R^{30b}, when present, is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; wherein each of R^{22a}, R^{22b}, R^{22c}, R^{22d}, R^{22e}, R^{22f}, R^{22g}, and R^{22f} is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino.

In a further aspect, the compound has a structure represented by a formula:

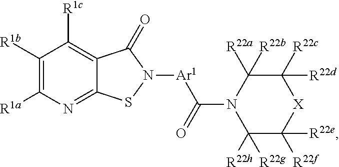

wherein X is selected from N, O, and CR^{30a}R^{30b}; wherein each of R^{30a} and R^{30b}, when present, is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; wherein each of R^{22a}, R^{22b}, R^{22c}, R^{22d}, R^{22e}, R^{22f}, R^{22g}, and R^{22f} is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino.

In a further aspect, the compound has a structure represented by a formula selected from:

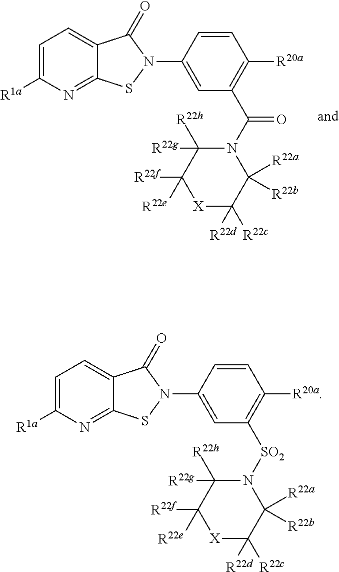

In a further aspect, the compound has a structure represented by a formula:

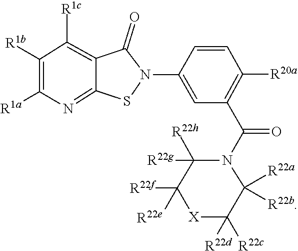

In a further aspect, the compound has a structure represented by a formula:

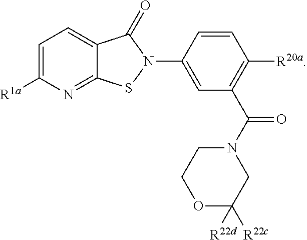

In a further aspect, the compound is selected from:

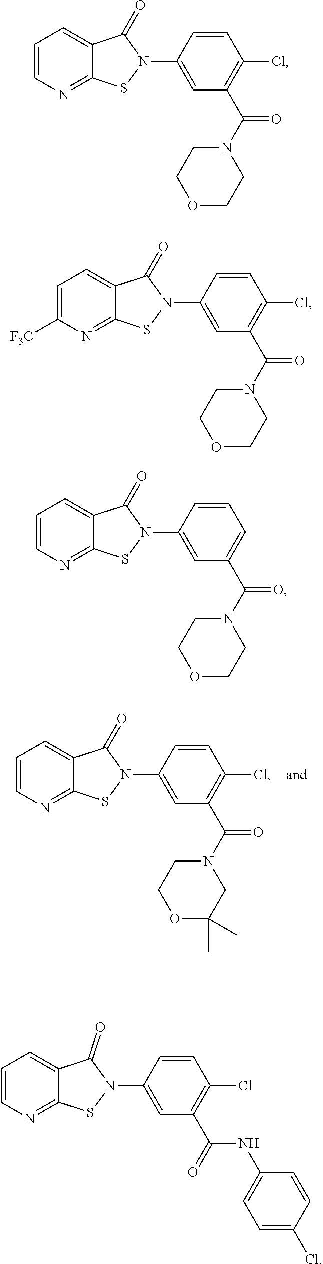 [SMILES: O=C(c1cc(-n2sc3ncccc3c2=O)ccc1Cl)N1CCOCC1 ; O=C(c1cc(-n2sc3nc(C(F)(F)F)ccc3c2=O)ccc1Cl)N1CCOCC1 ; O=C(c1cccc(-n2sc3ncccc3c2=O)c1)N1CCOCC1 ; CC1(C)CN(C(=O)c2cc(-n3sc4ncccc4c3=O)ccc2Cl)CCO1 ; "and" ; O=C(Nc1ccc(Cl)cc1)c1cc(-n2sc3ncccc3c2=O)ccc1Cl]

In one aspect, the compound inhibits Nef-Hck activity. In a further aspect, the compound inhibits in vitro NeF-Hck response. For example, the compound can have a NeF-Hck IC₅₀ of less than about 10 M, of less than about 5 M, of less than about 1 M, of less than about 500 nM, of less than about 100 nM, or of less than about 50 nM. In a further aspect, the compound can have a NeF-Hck IC₅₀ of less than about 10 M, of less than about 1 μM, of less than about 500 nM, of less than about 100 nM, of less than about 60 nM, or of less than about 20 nM.

It is understood that the disclosed compounds can be used in connection with the disclosed methods, compositions, kits, and uses.

a. L Groups

In one aspect, L is selected from CO and SO₂. In a further aspect, L is CO. In a still further aspect, L is SO₂.

b. X Groups

In one aspect, X is selected from N, O, and CR^{30a}R^{30b}. In a further aspect, X is selected from N and O. In a still further aspect, X is selected from N and CR^{30a}R^{30b}. In yet a further aspect, X is selected from O and CR^{30a}R^{30b}. In an even further aspect, X is N. In a still further aspect, X is O. In yet a further aspect, X is CR^{30a}R^{30b}.

In a further aspect, X is selected from N, O, and CH₂. In a still further aspect, X is selected from N and CH₂. In yet a further aspect, X is selected from O and CH₂. In an even further aspect, X is CH₂.

c. Z Groups

In one aspect, Z is selected from O and S. In a further aspect, Z is O. In a still further aspect, Z is S.

d. R^{1a}, R^{1b}, and R^{1c} Groups

In one aspect, each of R^{1a}, R^{1b}, and R^{1c} is independently selected from hydrogen, —NH₂, C1-C4 alkyl, C1-C4 haloalkyl, —CO₂H, —CO₂(C1-C4 alkyl), C1-C4 alkylamino, (C1-C4)(C1-C4) dialkylamino, and Cy¹. In a further aspect, each of R^{1a}, R^{1b}, and R^{1c} is hydrogen.

In a further aspect, each of R^{1a}, R^{1b}, and R^{1c} is independently selected from hydrogen-NH₂, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CO₂H, —CO₂CH₃, —CO₂CH₂CH₃, —CO₂CH₂CH₂CH₃, —CO₂CH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), and Cy¹. In a still further aspect, each of R^{1a}, R^{1b}, and R^{1c} is independently selected from hydrogen, —NH₂, methyl, ethyl, —CH₂F, —CH₂Cl, —CH₂CH₂F, —CH₂CH₂Cl, —CH(CH₃)CH₂Cl, —CO₂H, —CO₂CH₃, —CO₂CH₂CH₃, —NHCH₃, —NHCH₂CH₃, —N(CH₃)₂, —N(CH₂CH₃)₂, —N(CH₃)(CH₂CH₃), and Cy¹. In yet a further aspect, each of R^{1a}, R^{1b}, and R^{1c} is independently selected from hydrogen, —NH₂, methyl, —CH₂F, —CH₂Cl, —CO₂H, —CO₂CH₃, —NHCH₃, —N(CH₃)₂, and Cy¹.

In a further aspect, each of R^{1a}, R^{1b}, and R^{1c} is independently selected from hydrogen, C1-C4 alkyl, C1-C4 haloalkyl, —CO₂H, —CO₂(C1-C4 alkyl), C1-C4 alkylamino, (C1-C4)(C1-C4) dialkylamino, and Cy¹. In a still further aspect, each of R^{1a}, R^{1b}, and R^{1c} is independently selected from hydrogen, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CO₂H, —CO₂CH₃, —CO₂CH₂CH₃, —CO₂CH₂CH₂CH₃, —CO₂CH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), and Cy¹. In yet a further aspect, each of R^{1a}, R^{1b}, and R^{1c} is independently selected from hydrogen, methyl, ethyl, —CH₂F, —CH₂Cl, —CH₂CH₂F, —CH₂CH₂Cl, —CH(CH₃)CH₂Cl, —CO₂H, —CO₂CH₃, —CO₂CH₂CH₃, —NHCH₃, —NHCH₂CH₃, —N(CH₃)₂, —N(CH₂CH₃)₂, —N(CH₃)(CH₂CH₃), and Cy¹. In an even further aspect, each of R^{1a}, R^{1b}, and R^{1c} is independently selected from hydrogen, methyl, —CH₂F, —CH₂Cl, —CO₂H, —CO₂CH₃, —NHCH₃, —N(CH₃)₂, and Cy¹.

In a further aspect, each of R^{1a}, R^{1b}, and R^{1c} is independently selected from hydrogen, —NH₂, C1-C4 alkyl, C1-C4 haloalkyl, C1-C4 alkylamino, (C1-C4)(C1-C4) dialkylamino, and Cy¹. In a still further aspect, each of R^{1a}, R^{1b}, and R^{1c} is independently selected from hydrogen, —NH₂, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), and Cy¹. In a still further aspect, each of R^{1a}, R^{1b}, and R^{1c} is independently selected from hydrogen, —NH₂, methyl, ethyl, —CH₂F, —CH₂Cl, —CH₂CH₂F, —CH₂CH₂Cl, —CH(CH₃)CH₂Cl, —NHCH₃, —NHCH₂CH₃, —N(CH₃)₂, —N(CH₂CH₃)₂, —N(CH₃)(CH₂CH₃), and Cy¹. In yet a further aspect, each of R^{1a}, R^{1b}, and R^{1c} is independently selected from hydrogen, —NH₂, methyl, —CH₂F, —CH₂Cl, —NHCH₃, —N(CH₃)₂, and Cy¹.

In a further aspect, each of R^{1a}, R^{1b}, and R^{1c} is independently selected from hydrogen, —NH₂, C1-C4 alkyl, and Cy¹. In a still further aspect, each of R^{1a}, R^{1b}, and R^{1c} is independently selected from hydrogen, —NH₂, methyl, ethyl, n-propyl, i-propyl, and Cy¹. In a still further aspect, each of R^{1a}, R^{1b}, and R^{1c} is independently selected from hydrogen, —NH₂, methyl, ethyl, and Cy¹. In yet a further aspect, each of R^{1a}, R^{1b}, and R^{1c} is independently selected from hydrogen, —NH₂, methyl, and Cy¹.

In a further aspect, each of R^{1a}, R^{1b}, and R^{1c} is independently selected from hydrogen, —CO₂H, and —CO₂(C1-C4 alkyl). In a still further aspect, each of R^{1a}, R^{1b}, and R^{1c} is independently selected from hydrogen, —CO₂H, —CO₂CH₃, —CO₂CH₂CH₃, —CO₂CH₂CH₂CH₃, and —CO₂CH(CH₃)CH₃. In yet a further aspect, each of R^{1a}, R^{1b}, and R^{1c} is independently selected from hydrogen, —CO₂H, —CO₂CH₃, and —CO₂CH₂CH₃. In an even further aspect, each of R^{1a}, R^{1b}, and R^{1c} is independently selected from hydrogen, —CO₂H, and —CO₂CH₃.

In a further aspect, each of R^{1a}, R^{1b}, and R^{1c} is independently selected from hydrogen, C1-C4 alkyl, and C1-C4 haloalkyl. In a still further aspect, each of R^{1a}, R^{1b}, and R^{1c} is independently selected from hydrogen, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, and —CH(CH₃)CH₂Cl. In yet a further aspect, each of R^{1a}, R^{1b}, and R^{1c} is independently selected from hydrogen, methyl, ethyl, —CH₂F, —CH₂Cl, —CH₂CH₂F, and —CH₂CH₂Cl. In yet a further aspect, each of R^{1a}, R^{1b}, and R^{1c} is independently selected from hydrogen, methyl, —CH₂F, and —CH₂Cl.

In a further aspect, each of R^{1a}, R^{1b}, and R^{1c} is independently selected from hydrogen and Cy¹.

In a further aspect, each of R^{1a}, R^{1b}, and R^{1c} is independently selected from hydrogen and C1-C4 haloalkyl. In a still further aspect, each of R^{1a}, R^{1b}, and R^{1c} is independently selected from hydrogen, —CH₂F, —CH₂Cl, —CH₂CH₂F, —CH₂CH₂Cl, —CH₂CH₂CH₂F, —CH₂CH₂CH₂Cl, —CH(CH₃)CH₂F, and —CH(CH₃)CH₂Cl. In yet a further aspect, each of R^{1a}, R^{1b}, and R^{1c} is independently selected from hydrogen, —CH₂F, —CH₂Cl, —CH₂CH₂F, and —CH₂CH₂Cl. In yet a further aspect, each of R^{1a}, R^{1b}, and R^{1c} is independently selected from hydrogen, —CH₂F and —CH₂Cl.

In a further aspect, each of R^{1a}, R^{1b}, and R^{1c} is independently selected from hydrogen and C1-C4 alkyl. In a still further aspect, each of R^{1a}, R^{1b}, and R^{1c} is independently selected from hydrogen, methyl, ethyl, n-propyl, i-propyl, n-butyl, i-butyl, s-butyl, and t-butyl. In yet a further aspect, each of R^{1a}, R^{1b}, and R^{1c} is independently selected from hydrogen, methyl, ethyl, n-propyl, and i-propyl. In an even further aspect, each of R^{1a}, R^{1b}, and R^{1c} is independently selected from hydrogen, methyl and ethyl. In a still further aspect, each of R^{1a}, R^{1b}, and R^{1c} is independently selected from hydrogen and ethyl. In yet a further aspect, each of R^{1a}, R^{1b}, and R^{1c} is independently selected from hydrogen and methyl.

In a further aspect, each of R^{1a}, R^{1b}, and R^{1c} is independently selected from hydrogen, —NH₂, C1-C4 alkyl, C1-C4 alkylamino, and (C1-C4)(C1-C4) dialkylamino. In a further aspect, each of R^{1a}, R^{1b}, and R^{1c} is independently selected from hydrogen, —NH₂, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{1a}, R^{1b}, and R^{1c} is independently selected from hydrogen, —NH₂, methyl, ethyl, —CH₂F, —CH₂Cl, —CH₂CH₂F, —CH₂CH₂Cl, —NHCH₃, —NHCH₂CH₃, —N(CH₃)₂, —N(CH₂CH₃)₂, and —N(CH₃)(CH₂CH₃). In yet a further aspect, each of R^{1a}, R^{1b}, and R^{1c} is independently selected from hydrogen, —NH₂, methyl, —CH₂F, —CH₂Cl, —NHCH₃, and —N(CH₃)₂.

In a further aspect, each of R^{1a}, R^{1b}, and R^{1c} is independently selected from hydrogen, —NH₂, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), and Cy¹. In a still further aspect, each of R^{1a}, R^{1b}, and R^{1c} is independently selected from hydrogen, —NH₂, methyl, ethyl, —CH₂F, —CH₂Cl, —CH₂CH₂F, —CH₂CH₂Cl, —NHCH₃, —NHCH₂CH₃, —N(CH₃)₂, —N(CH₂CH₃)₂, —N(CH₃)(CH₂CH₃), and Cy¹. In yet a further aspect, each of R^{1a}, R^{1b}, and R^{1c} is independently selected from hydrogen, —NH₂, methyl, —CH₂F, —CH₂Cl, —NHCH₃, —N(CH₃)₂, and Cy¹.

e. R² Groups

In one aspect, each occurrence of R², when present, is independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl), or wherein any two adjacent R² groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl).

In a further aspect, each occurrence of R², when present, is independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, each occurrence of R², when present, is independently selected from —F, —Cl, —CN, —NH₂, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —CO₂H, —CO₂CH₃, —CO₂CH₂CH₃, —CO₂CH₂CH₂CH₃, —CO₂CH(CH₃)CH₃, —SO₂H, —SO₂CH₃, —SO₂CH₂CH₃, —SO₂CH₂CH₂CH₃, —SO₂CH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CO₂NH₂, —CO₂NHCH₃, —CO₂NHCH₂CH₃, —CO₂NHCH₂CH₂CH₃, —CO₂NHCH(CH₃)CH₃, —CO₂N(CH₃)₂, —CO₂N(CH₂CH₃)₂, —CO₂N(CH₂CH₂CH₃)₂, —CO₂N(CH(CH₃)CH₃)₂, —CO₂N(CH₃)(CH₂CH₃), —SO₂NH₂, —SO₂NHCH₃, —SO₂NHCH₂CH₃, —SO₂NHCH₂CH₂CH₃, —SO₂NHCH(CH₃)CH₃, —SO₂N(CH₃)₂, —SO₂N(CH₂CH₃)₂, —SO₂N(CH₂CH₂CH₃)₂, —SO₂N(CH(CH₃)CH₃)₂, and —SO₂N(CH₃)(CH₂CH₃). In yet a further aspect, each occurrence of R², when present, is independently selected from —F, —Cl, —CN, —NH₂, —OH, methyl, ethyl, —CH₂F, —CH₂Cl, —CH₂CH₂F, —CH₂CH₂Cl, —CH₂OH, —CH₂CH₂OH, —OCH₃, —OCH₂CH₃, —CO₂H, —CO₂CH₃, —CO₂CH₂CH₃, —SO₂H, —SO₂CH₃, —SO₂CH₂CH₃, —NHCH₃, —NHCH₂CH₃, —N(CH₃)₂, —N(CH₂CH₃)₂, —N(CH₃)(CH₂CH₃), —CO₂NH₂, —CO₂NHCH₃, —CO₂NHCH₂CH₃, —CO₂N(CH₃)₂, —CO₂N(CH₂CH₃)₂, —CO₂N(CH₃)(CH₂CH₃), —SO₂NH₂, —SO₂NHCH₃, —SO₂NHCH₂CH₃, —SO₂N(CH₃)₂, —SO₂N(CH₂CH₃)₂, and —SO₂N(CH₃)(CH₂CH₃). In an even further aspect, each occurrence of R², when present, is independently selected from —F, —Cl, —CN, —NH₂, —OH, methyl, —CH₂F, —CH₂Cl, —CH₂OH, —OCH₃, —CO₂H, —CO₂CH₃, —SO₂H, —SO₂CH₃, —NHCH₃, —N(CH₃)₂, —CO₂NH₂, —C₂NHCH₃, —CO₂N(CH₃)₂, —SO₂NH₂, —SO₂NHCH₃, and —SO₂N(CH₃)₂.

In a further aspect, each occurrence of R², when present, is independently selected from halogen, —CN, —NH₂, —OH, C1-C4 alkyl, C1-C4 haloalkyl, C1-C4 hydroxyalkyl, and C1-C4 alkoxy. In a still further aspect, each occurrence of R², when present, is independently selected from —F, —Cl, —CN, —NH₂, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In yet a further aspect, each occurrence of R², when present, is independently selected from —F, —Cl, —CN, —NH₂, —OH, methyl, ethyl, —CH₂F, —CH₂Cl, —CH₂CH₂F, —CH₂CH₂Cl, —CH₂OH, —CH₂CH₂OH, —OCH₃, and —OCH₂CH₃. In an even further aspect, each occurrence of R², when present, is independently selected from —F, —Cl, —CN, —NH₂, —OH, methyl, —CH₂F, —CH₂Cl, —CH₂OH, and —OCH₃.

In a further aspect, each occurrence of R², when present, is independently selected from —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, each occurrence of R², when present, is independently selected from —CO₂H, —CO₂CH₃, —CO₂CH₂CH₃, —CO₂CH₂CH₂CH₃, —CO₂CH(CH₃)CH₃, —SO₂H, —SO₂CH₃, —SO₂CH₂CH₃, —SO₂CH₂CH₂CH₃, —SO₂CH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CO₂NH₂, —CO₂NHCH₃, —CO₂NHCH₂CH₃, —CO₂NHCH₂CH₂CH₃, —CO₂NHCH(CH₃)CH₃, —CO₂N(CH₃)₂, —CO₂N(CH₂CH₃)₂, —CO₂N(CH₂CH₂CH₃)₂, —CO₂N(CH(CH₃)CH₃)₂, —CO₂N(CH₃)(CH₂CH₃), —SO₂NH₂, —SO₂NHCH₃, —SO₂NHCH₂CH₃, —SO₂NHCH₂CH₂CH₃, —SO₂NHCH(CH₃)CH₃, —SO₂N(CH₃)₂, —SO₂N(CH₂CH₃)₂, —SO₂N(CH₂CH₂CH₃)₂, —SO₂N(CH(CH₃)CH₃)₂, and —SO₂N(CH₃)(CH₂CH₃). In yet a further aspect, each occurrence of R², when present, is independently selected from —CO₂H, —CO₂CH₃, —CO₂CH₂CH₃, —SO₂H, —SO₂CH₃, —SO₂CH₂CH₃, —NHCH₃, —NHCH₂CH₃, —N(CH₃)₂, —N(CH₂CH₃)₂, —N(CH₃)(CH₂CH₃), —CO₂NH₂, —CO₂NHCH₃, —CO₂NHCH₂CH₃, —CO₂N(CH₃)₂, —CO₂N(CH₂CH₃)₂, —CO₂N(CH₃)(CH₂CH₃), —SO₂NH₂, —SO₂NHCH₃, —SO₂NHCH₂CH₃, —SO₂N(CH₃)₂, —SO₂N(CH₂CH₃)₂, and —SO₂N(CH₃)(CH₂CH₃). In an even further aspect, each occurrence of R², when present, is independently selected from —CO₂H, —CO₂CH₃, —SO₂H, —SO₂CH₃, —NHCH₃, —N(CH₃)₂, —CO₂NH₂, —CO₂NHCH₃, —CO₂N(CH₃)₂, —SO₂NH₂, —SO₂NHCH₃, and —SO₂N(CH₃)₂.

In a further aspect, each occurrence of R², when present, is independently selected from halogen and C1-C4 alkyl. In a still further aspect, each occurrence of R², when present, is independently selected from —F, —Cl, methyl, ethyl, n-propyl, and i-propyl. In yet a further aspect, each occurrence of R², when present, is independently selected from —F, —Cl, methyl, and ethyl. In an even further aspect, each occurrence of R², when present, is independently selected from —F, —Cl, and methyl.

In a further aspect, each occurrence of R², when present, is independently halogen. In a still further aspect, each occurrence of R², when present, is independently selected from —Br, —F, and —Cl. In yet a further aspect, each occurrence of R², when present, is independently selected from —F and —Cl. In an even further aspect, each occurrence of R², when present, is —F. In a still further aspect, each occurrence of R², when present, is —Cl.

In a further aspect, each occurrence of R², when present, is independently C1-C4 alkyl. In a still further aspect, each occurrence of R², when present, is independently selected from methyl, ethyl, n-propyl, and i-propyl. In yet a further aspect, each occurrence of R², when present, is independently selected from methyl and ethyl. In an even further aspect, each occurrence of R², when present, is ethyl. In a still further aspect, each occurrence of R², when present, is methyl.

In a further aspect, two adjacent R² groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 5- to 6-membered aryl or a 5- to 6-membered heteroaryl, and are substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R² groups are covalently bonded together and, together with the intermediate atoms, comprise a 5- to 6-membered aryl or a 5- to 6-membered heteroaryl, and are substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R² groups are covalently bonded together and, together with the intermediate atoms, comprise a 5- to 6-membered aryl or a 5- to 6-membered heteroaryl, and are monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 5- to 6-membered aryl or a 5- to 6-membered heteroaryl, and are unsubstituted.

In a further aspect, two adjacent R² groups are covalently bonded together and, together with the intermediate atoms, comprise a 5- to 6-membered aryl substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 5- to 6-membered aryl substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R² groups are covalently bonded together and, together with the intermediate atoms, comprise a 5- to 6-membered aryl substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R² groups are covalently bonded together and, together with the intermediate atoms, comprise a 5- to 6-membered aryl monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise an unsubstituted 5- to 6-membered aryl.

In a further aspect, two adjacent R² groups are covalently bonded together and, together with the intermediate atoms, comprise a 5-membered aryl substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 5-membered aryl substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R² groups are covalently bonded together and, together with the intermediate atoms, comprise a 5-membered aryl substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R² groups are covalently bonded together and, together with the intermediate atoms, comprise a 5-membered aryl monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise an unsubstituted 5-membered aryl.

In a further aspect, two adjacent R² groups are covalently bonded together and, together with the intermediate atoms, comprise a 6-membered aryl substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 6-membered aryl substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R² groups are covalently bonded together and, together with the intermediate atoms, comprise a 6-membered aryl substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R² groups are covalently bonded together and, together with the intermediate atoms, comprise a 6-membered aryl monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise an unsubstituted 6-membered aryl.

In a further aspect, two adjacent R² groups are covalently bonded together and, together with the intermediate atoms, comprise a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 5- to 6-membered heteroaryl, and are substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R² groups are covalently bonded together and, together with the intermediate atoms, comprise a 5- to 6-membered heteroaryl, and are substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R² groups are covalently bonded together and, together with the intermediate atoms, comprise a 5- to 6-membered heteroaryl, and are monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 5- to 6-membered heteroaryl, and are unsubstituted.

In a further aspect, two adjacent R² groups are covalently bonded together and, together with the intermediate atoms, comprise a 5-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 5-membered heteroaryl, and are substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R² groups are covalently bonded together and, together with the intermediate atoms, comprise a 5-membered heteroaryl, and are substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R² groups are covalently bonded together and, together with the intermediate atoms, comprise a 5-membered heteroaryl, and are monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 5-membered heteroaryl, and are unsubstituted.

In a further aspect, two adjacent R² groups are covalently bonded together and, together with the intermediate atoms, comprise a 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 6-membered heteroaryl, and are substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R² groups are covalently bonded together and, together with the intermediate atoms, comprise a 6-membered heteroaryl, and are substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R² groups are covalently bonded together and, together with the intermediate atoms, comprise a 6-membered heteroaryl, and are monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R² groups are covalently bonded together and, together with the intermediate atoms, comprise a 6-membered heteroaryl, and are unsubstituted.

f. R³ Groups

In one aspect, R³ is hydrogen or C1-C4 alkyl or wherein each of R³ and R⁴ are covalently bonded together and, together with the intermediate atoms, comprise a 3- to 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino.

In a further aspect, R³ is hydrogen or C1-C4 alkyl. In a still further aspect, R³ is hydrogen.

In a further aspect, R³ is hydrogen, methyl, ethyl, n-propyl, i-propyl, n-butyl, i-butyl, s-butyl, or t-butyl. In a still further aspect, R³ is hydrogen, methyl, ethyl, n-propyl, or i-propyl. In yet a further aspect, R³ is hydrogen, methyl, or ethyl. In an even further aspect, R³ is hydrogen or ethyl. In a still further aspect, R³ is hydrogen or methyl.

In a further aspect, R³ is methyl, ethyl, n-propyl, i-propyl, n-butyl, i-butyl, s-butyl, or t-butyl. In a still further aspect, R³ is methyl, ethyl, n-propyl, or i-propyl. In yet a further aspect, R³ is methyl or ethyl. In an even further aspect, R³ is ethyl. In a still further aspect, R³ is methyl.

In a further aspect, each of R³ and R⁴ are covalently bonded together and, together with the intermediate atoms, comprise a 3- to 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise a 3- to 6-membered heterocycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, each of R³ and R⁴ are covalently bonded together and, together with the intermediate atoms, comprise a 3- to 6-membered heterocycloalkyl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, each of R³ and R⁴ are covalently bonded together and, together with the intermediate atoms, comprise a 3- to 6-membered heterocycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise an unsubstituted 3- to 6-membered heterocycloalkyl.

In a further aspect, each of R³ and R⁴ are covalently bonded together and, together with the intermediate atoms, comprise a 3-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise a 3-membered heterocycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, each of R³ and R⁴ are covalently bonded together and, together with the intermediate atoms, comprise a 3-membered heterocycloalkyl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, each of R³ and R⁴ are covalently bonded together and, together with the intermediate atoms, comprise a 3-membered heterocycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise an unsubstituted 3-membered heterocycloalkyl.

In a further aspect, each of R³ and R⁴ are covalently bonded together and, together with the intermediate atoms, comprise a 4-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise a 4-membered heterocycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, each of R³ and R⁴ are covalently bonded together and, together with the intermediate atoms, comprise a 4-membered heterocycloalkyl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, each of R³ and R⁴ are covalently bonded together and, together with the intermediate atoms, comprise a 4-membered heterocycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise an unsubstituted 4-membered heterocycloalkyl.

In a further aspect, each of R³ and R⁴ are covalently bonded together and, together with the intermediate atoms, comprise a 5-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise a 5-membered heterocycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, each of R³ and R⁴ are covalently bonded together and, together with the intermediate atoms, comprise a 5-membered heterocycloalkyl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, each of R³ and R⁴ are covalently bonded together and, together with the intermediate atoms, comprise a 5-membered heterocycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise an unsubstituted 5-membered heterocycloalkyl.

In a further aspect, each of R³ and R⁴ are covalently bonded together and, together with the intermediate atoms, comprise a 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise a 6-membered heterocycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, each of R³ and R⁴ are covalently bonded together and, together with the intermediate atoms, comprise a 6-membered heterocycloalkyl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, each of R³ and R⁴ are covalently bonded together and, together with the intermediate atoms, comprise a 6-membered heterocycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R³ and R⁴ are covalently bonded together and, together with the intermediate atoms, comprise an unsubstituted 6-membered heterocycloalkyl.

g. R⁴ Groups

In one aspect, R⁴ is selected from C1-C8 alkyl, Cy², and (C1-C4)Cy². In a further aspect, R⁴ is selected from C1-C4 alkyl, Cy², and (C1-C4)Cy². In a still further aspect, R⁴ is selected from methyl, ethyl, n-propyl, i-propyl, Cy², —CH₂Cy², —CH₂CH₂Cy², —CH₂CH₂CH₂Cy², and —CH(CH₃)CH₂Cy². In yet a further aspect, R⁴ is selected from methyl, ethyl, Cy², —CH₂Cy², and —CH₂CH₂Cy². In an even further aspect, R⁴ is selected from methyl, Cy², and —CH₂Cy².

In a further aspect, R⁴ is Cy².

In a further aspect, R⁴ is selected from C1-C8 alkyl and Cy². In a still further aspect, R⁴ is selected from C1-C4 alkyl and Cy². In yet a further aspect, R⁴ is selected from methyl, ethyl, n-propyl, i-propyl, and Cy². In yet a further aspect, R⁴ is selected from methyl, ethyl, and Cy². In an even further aspect, R⁴ is selected from methyl and Cy².

In a further aspect, R⁴ is selected from C1-C8 alkyl and (C1-C4)Cy². In a still further aspect, R⁴ is selected from C1-C4 alkyl and (C1-C4)Cy². In yet a further aspect, R⁴ is selected from methyl, ethyl, n-propyl, i-propyl, —CH₂Cy², —CH₂CH₂Cy², —CH₂CH₂CH₂Cy², and —CH(CH₃) CH₂Cy². In an even further aspect, R⁴ is selected from methyl, ethyl, —CH₂Cy², and —CH₂CH₂Cy². In a still further aspect, R⁴ is selected from methyl and —CH₂Cy².

In a further aspect, R⁴ is selected from Cy² and (C1-C4)Cy². In a still further aspect, R⁴ is selected from Cy², —CH₂Cy², —CH₂CH₂Cy², —CH₂CH₂CH₂Cy², and —CH(CH₃) CH₂Cy². In yet a further aspect, R⁴ is selected from Cy², —CH₂Cy², and —CH₂CH₂Cy². In an even further aspect, R⁴ is selected from Cy² and —CH₂Cy².

In a further aspect, R⁴ is (C1-C4)Cy². In a still further aspect, R⁴ is selected from —CH₂Cy², —CH₂CH₂Cy², —CH₂CH₂CH₂Cy², and —CH(CH₃) CH₂Cy². In yet a further aspect, R⁴ is selected from —CH₂Cy² and —CH₂CH₂Cy². In an even further aspect, R⁴ is —CH₂Cy². In a still further aspect, R⁴ is —CH₂CH₂Cy².

In a further aspect, R⁴ is C1-C8 alkyl. In a still further aspect, R⁴ is C1-C4 alkyl. In yet a further aspect, R⁴ is selected from methyl, ethyl, n-propyl, and i-propyl. In an even further aspect, R⁴ is selected from methyl and ethyl. In a still further aspect, R⁴ is ethyl. In yet a further aspect, R⁴ is methyl.

h. R^{20a}, R^{20b}, R^{20c}, and R^{20d} Groups

In one aspect, each of R^{20a}, R^{20b}, R^{20c}, and R^{20d} is independently selected from hydrogen, halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl); or wherein any two adjacent R^{20a}, R^{20b}, R^{20c}, and R^{20d} groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a further aspect, each of R^{20a}, R^{20b}, R^{20c}, and R^{20d} is hydrogen.

In a further aspect, each of R^{20a}, R^{20b}, R^{20c}, and R^{20d} is independently selected from hydrogen, halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, each of R^{20a}, R^{20b}, R^{20c}, and R^{20d} is independently selected from hydrogen, —F, —Cl, —CN, —NH₂, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —CO₂H, —CO₂CH₃, —CO₂CH₂CH₃, —CO₂CH₂CH₂CH₃, —CO₂CH(CH₃)CH₃, —SO₂H, —SO₂CH₃, —SO₂CH₂CH₃, —SO₂CH₂CH₂CH₃, —SO₂CH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CO₂NH₂, —CO₂NHCH₃, —CO₂NHCH₂CH₃, —CO₂NHCH₂CH₂CH₃, —CO₂NHCH(CH₃)CH₃, —CO₂N(CH₃)₂, —CO₂N(CH₂CH₃)₂, —CO₂N(CH₂CH₂CH₃)₂, —CO₂N(CH(CH₃)CH₃)₂, —CO₂N(CH₃)(CH₂CH₃), —SO₂NH₂, —SO₂NHCH₃, —SO₂NHCH₂CH₃, —SO₂NHCH₂CH₂CH₃, —SO₂NHCH(CH₃)CH₃, —SO₂N(CH₃)₂, —SO₂N(CH₂CH₃)₂, —SO₂N(CH₂CH₂CH₃)₂, —SO₂N(CH(CH₃)CH₃)₂, and —SO₂N(CH₃)(CH₂CH₃). In yet a further aspect, each of R^{20a}, R^{20b}, R^{20c}, and R^{20d} is independently selected from hydrogen, —F, —Cl, —CN, —NH₂, —OH, methyl, ethyl, —CH₂F, —CH₂Cl, —CH₂CH₂F, —CH₂CH₂Cl, —CH₂OH, —CH₂CH₂OH, —OCH₃, —OCH₂CH₃, —CO₂H, —CO₂CH₃, —CO₂CH₂CH₃, —SO₂H, —SO₂CH₃, —SO₂CH₂CH₃, —NHCH₃, —NHCH₂CH₃, —N(CH₃)₂, —N(CH₂CH₃)₂, —N(CH₃)(CH₂CH₃), —CO₂NH₂, —CO₂NHCH₃, —CO₂NHCH₂CH₃, —CO₂N(CH₃)₂, —CO₂N(CH₂CH₃)₂, —CO₂N(CH₃)(CH₂CH₃), —SO₂NH₂, —SO₂NHCH₃, —SO₂NHCH₂CH₃, —SO₂N(CH₃)₂, —SO₂N(CH₂CH₃)₂, and —SO₂N(CH₃)(CH₂CH₃). In an even further aspect, each of R^{20a}, R^{20b}, R^{20c}, and R^{20d} is independently selected from hydrogen, —F, —Cl, —CN, —NH₂, —OH, methyl, —CH₂F, —CH₂Cl, —CH₂OH, —OCH₃, —CO₂H, —CO₂CH₃, —SO₂H, —SO₂CH₃, —NHCH₃, —N(CH₃)₂, —CO₂NH₂, —CO₂NHCH₃, —CO₂N(CH₃)₂, —SO₂NH₂, —SO₂NHCH₃, and —SO₂N(CH₃)₂.

In a further aspect, each of R^{20a}, R^{20b}, R^{20c}, and R^{20d} is independently selected from hydrogen, halogen, —CN, —NH₂, —OH, C1-C4 alkyl, C1-C4 haloalkyl, C1-C4 hydroxyalkyl, and C1-C4 alkoxy. In a still further aspect, each of R^{20a}, R^{20b}, R^{20c}, and R^{20d} is independently selected from hydrogen, —F, —Cl, —CN, —NH₂, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In yet a further aspect, each of R^{20a}, R^{20b}, R^{20c}, and R^{20d} is independently selected from hydrogen, —F, —Cl, —CN, —NH₂, —OH, methyl, ethyl, —CH₂F, —CH₂Cl, —CH₂CH₂F, —CH₂CH₂Cl, —CH₂OH, —CH₂CH₂OH, —OCH₃, and —OCH₂CH₃. In an even further aspect, each of R^{20a}, R^{20b}, R^{20c}, and R^{20d} is independently selected from hydrogen, —F, —Cl, —CN, —NH₂, —OH, methyl, —CH₂F, —CH₂Cl, —CH₂OH, and —OCH₃.

In a further aspect, each of R^{20a}, R^{20b}, R^{20c}, and R^{20d} is independently selected from hydrogen, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, each of R^{20a}, R^{20b}, R^{20c}, and R^{20d} is independently selected from hydrogen, —CO₂H, —CO₂CH₃, —CO₂CH₂CH₃, —CO₂CH₂CH₂CH₃, —CO₂CH(CH₃)CH₃, —SO₂H, —SO₂CH₃, —SO₂CH₂CH₃, —SO₂CH₂CH₂CH₃, —SO₂CH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CO₂NH₂, —CO₂NHCH₃, —CO₂NHCH₂CH₃, —CO₂NHCH₂CH₂CH₃, —CO₂NHCH(CH₃)CH₃, —CO₂N(CH₃)₂, —CO₂N(CH₂CH₃)₂, —CO₂N(CH₂CH₂CH₃)₂, —CO₂N(CH(CH₃)CH₃)₂, —CO₂N(CH₃)(CH₂CH₃), —SO₂NH₂, —SO₂NHCH₃, —SO₂NHCH₂CH₃, —SO₂NHCH₂CH₂CH₃, —SO₂NHCH(CH₃)CH₃, —SO₂N(CH₃)₂, —SO₂N(CH₂CH₃)₂, —SO₂N(CH₂CH₂CH₃)₂, —SO₂N(CH(CH₃)CH₃)₂, and —SO₂N(CH₃)(CH₂CH₃). In yet a further aspect, each of R^{20a}, R^{20b}, R^{20c}, and R^{20d} is independently selected from hydrogen, —CO₂H, —CO₂CH₃, —CO₂CH₂CH₃, —SO₂H, —SO₂CH₃, —SO₂CH₂CH₃, —NHCH₃, —NHCH₂CH₃, —N(CH₃)₂, —N(CH₂CH₃)₂, —N(CH₃)(CH₂CH₃), —CO₂NH₂, —CO₂NHCH₃, —CO₂NHCH₂CH₃, —CO₂N(CH₃)₂, —CO₂N(CH₂CH₃)₂, —CO₂N(CH₃)(CH₂CH₃), —SO₂NH₂, —SO₂NHCH₃, —SO₂NHCH₂CH₃, —SO₂N(CH₃)₂, —SO₂N(CH₂CH₃)₂, and —SO₂N(CH₃)(CH₂CH₃). In an even further aspect, each of R^{20a}, R^{20b}, R^{20c}, and R^{20d} is independently selected from hydrogen, —CO₂H, —CO₂CH₃, —SO₂H, —SO₂CH₃, —NHCH₃, —N(CH₃)₂, —CO₂NH₂, —CO₂NHCH₃, —CO₂N(CH₃)₂, —SO₂NH₂, —SO₂NHCH₃, and —SO₂N(CH₃)₂.

In a further aspect, each of R^{20a}, R^{20b}, R^{20c}, and R^{20d} is independently selected from hydrogen, halogen, and C1-C4 alkyl. In a still further aspect, each of R^{20a}, R^{20b}, R^{20c}, and R^{20d} is independently selected from hydrogen, —F, —Cl, methyl, ethyl, n-propyl, and i-propyl. In yet a further aspect, each of R^{20a}, R^{20b}, R^{20c}, and R^{20d} is independently selected from hydrogen, —F, —Cl, methyl, and ethyl. In an even further aspect, each of R^{20a}, R^{20b}, R^{20c}, and R^{20d} is independently selected from hydrogen, —F, —Cl, and methyl.

In a further aspect, each of R^{20a}, R^{20b}, R^{20c}, and R^{20d} is independently selected from hydrogen and halogen. In a still further aspect, each of R^{20a}, R^{20b}, R^{20c}, and R^{20d} is independently selected from hydrogen, —Br, —F, and —Cl. In yet a further aspect, each of R^{20a}, R^{20b}, R^{20c}, and R^{20d} is independently selected from hydrogen, —F, and —Cl. In an even further aspect, each of R^{20a}, R^{20b}, R^{20c}, and R^{20d} is independently selected from hydrogen and —F. In a still further aspect, each of R^{20a}, R^{20b}, R^{20c}, and R^{20d} is independently selected from hydrogen and —Cl.

In a further aspect, each of R^{20a}, R^{20b}, R^{20c}, and R^{20d} is independently selected from hydrogen and C1-C4 alkyl. In a still further aspect, each of R^{20a}, R^{20b}, R^{20c}, and R^{20d} is independently selected from hydrogen, methyl, ethyl, n-propyl, and i-propyl. In yet a further aspect, each of R^{20a}, R^{20b}, R^{20c}, and R^{20d} is independently selected from hydrogen, methyl, and ethyl. In an even further aspect, each of R^{20a}, R^{20b}, R^{20c}, and R^{20d} is independently selected from hydrogen and ethyl. In a still further aspect, each of R^{20a}, R^{20b}, R^{20c}, and R^{20d} is independently selected from hydrogen and methyl.

In a further aspect, each of R^{20b}, R^{20c}, and R^{20d} is hydrogen and R^{20a} is selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, each of R^{20b}, R^{20c}, and R^{20d} is hydrogen and R^{20a} is selected from —F, —Cl, —CN, —NH₂, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —CO₂H, —CO₂CH₃, —CO₂CH₂CH₃, —CO₂CH₂CH₂CH₃, —CO₂CH(CH₃)CH₃, —SO₂H, —SO₂CH₃, —SO₂CH₂CH₃, —SO₂CH₂CH₂CH₃, —SO₂CH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CO₂NH₂, —CO₂NHCH₃, —CO₂NHCH₂CH₃, —CO₂NHCH₂CH₂CH₃, —CO₂NHCH(CH₃)CH₃, —CO₂N(CH₃)₂, —CO₂N(CH₂CH₃)₂, —CO₂N(CH₂CH₂CH₃)₂, —CO₂N(CH(CH₃)CH₃)₂, —CO₂N(CH₃)(CH₂CH₃), —SO₂NH₂, —SO₂NHCH₃, —SO₂NHCH₂CH₃, —SO₂NHCH₂CH₂CH₃, —SO₂NHCH(CH₃)CH₃, —SO₂N(CH₃)₂, —SO₂N(CH₂CH₃)₂, —SO₂N(CH₂CH₂CH₃)₂, —SO₂N(CH(CH₃)CH₃)₂, and —SO₂N(CH₃)(CH₂CH₃). In yet a further aspect, each of R^{20b}, R^{20c}, and R^{20d} is hydrogen and R^{20a} is selected from —F, —Cl, —CN, —NH₂, —OH, methyl, ethyl, —CH₂F, —CH₂Cl, —CH₂CH₂F, —CH₂CH₂Cl, —CH₂OH, —CH₂CH₂OH, —OCH₃, —OCH₂CH₃, —CO₂H, —CO₂CH₃, —CO₂CH₂CH₃, —SO₂H, —SO₂CH₃, —SO₂CH₂CH₃, —NHCH₃, —NHCH₂CH₃, —N(CH₃)₂, —N(CH₂CH₃)₂, —N(CH₃)(CH₂CH₃), —CO₂NH₂, —CO₂NHCH₃, —CO₂NHCH₂CH₃, —CO₂N(CH₃)₂, —CO₂N(CH₂CH₃)₂, —CO₂N(CH₃)(CH₂CH₃), —SO₂NH₂, —SO₂NHCH₃, —SO₂NHCH₂CH₃, —SO₂N(CH₃)₂, —SO₂N(CH₂CH₃)₂, and —SO₂N(CH₃)(CH₂CH₃). In an even further aspect, each of R^{20b}, R^{20c}, and R^{20d} is hydrogen and R^{20a} is selected from —F, —Cl, —CN, —NH₂, —OH, methyl, —CH₂F, —CH₂Cl, —CH₂OH, —OCH₃, —CO₂H, —CO₂CH₃, —SO₂H, —SO₂CH₃, —NHCH₃, —N(CH₃)₂, —CO₂NH₂, —CO₂NHCH₃, —CO₂N(CH₃)₂, —SO₂NH₂, —SO₂NHCH₃, and —SO₂N(CH₃)₂.

In a further aspect, each of R^{20b}, R^{20c}, and R^{20d} is hydrogen and R^{20a} is halogen. In a still further aspect, each of R^{20b}, R^{20c}, and R^{20d} is hydrogen and R^{20a} is selected from —Br, —Cl, and —F. In yet a further aspect, each of R^{20b}, R^{20c}, and R^{20d} is hydrogen and R^{20a} is selected from —Cl and —F. In an even further aspect, each of R^{20b}, R^{20c}, and R^{20d} is hydrogen and R^{20a} is —Cl. In a still further aspect, each of R^{20b}, R^{20c}, and R^{20d} is hydrogen and R^{20a} is —F.

In a further aspect, two adjacent R^{20a}, R^{20b}, R^{20c}, and R^{20d} groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 5- to 6-membered aryl or a 5- to 6-membered heteroaryl, and are substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R^{20a}, R^{20b}, R^{20c}, and R^{20d} groups are covalently bonded together and, together with the intermediate atoms, comprise a 5- to 6-membered aryl or a 5- to 6-membered heteroaryl, and are substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R^{20a}, R^{20b}, R^{20c}, and R^{20d} groups are covalently bonded together and, together with the intermediate atoms, comprise a 5- to 6-membered aryl or a 5- to 6-membered heteroaryl, and are monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 5- to 6-membered aryl or a 5- to 6-membered heteroaryl, and are unsubstituted.

In a further aspect, two adjacent R^{20a}, R^{20b}, R^{20c}, and R^{20d} groups are covalently bonded together and, together with the intermediate atoms, comprise a 5- to 6-membered aryl substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 5- to 6-membered aryl substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R^{20a}, R^{20b}, R^{20c}, and R^{20d} groups are covalently bonded together and, together with the intermediate atoms, comprise a 5- to 6-membered aryl substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R^{20a}, R^{20b}, R^{20c}, and R^{20d} groups are covalently bonded together and, together with the intermediate atoms, comprise a 5- to 6-membered aryl monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise an unsubstituted 5- to 6-membered aryl.

In a further aspect, two adjacent R^{20a}, R^{20b}, R^{20c}, and R^{20d} groups are covalently bonded together and, together with the intermediate atoms, comprise a 5-membered aryl substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 5-membered aryl substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R^{20a}, R^{20b}, R^{20c}, and R^{20d} groups are covalently bonded together and, together with the intermediate atoms, comprise a 5-membered aryl substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R^{20a}, R^{20b}, R^{20c}, and R^{20d} groups are covalently bonded together and, together with the intermediate atoms, comprise a 5-membered aryl monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise an unsubstituted 5-membered aryl.

In a further aspect, two adjacent R^{20a}, R^{20b}, R^{20c}, and R^{20d} groups are covalently bonded together and, together with the intermediate atoms, comprise a 6-membered aryl substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 6-membered aryl substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R^{20a}, R^{20b}, R^{20c}, and R^{20d} groups are covalently bonded together and, together with the intermediate atoms, comprise a 6-membered aryl substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R^{20a}, R^{20b}, R^{20c}, and R^{20d} groups are covalently bonded together and, together with the intermediate atoms, comprise a 6-membered aryl monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise an unsubstituted 6-membered aryl.

In a further aspect, two adjacent R^{20a}, R^{20b}, R^{20c}, and R^{20d} groups are covalently bonded together and, together with the intermediate atoms, comprise a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 5- to 6-membered heteroaryl, and are substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R^{20a}, R^{20b}, R^{20c}, and R^{20d} groups are covalently bonded together and, together with the intermediate atoms, comprise a 5- to 6-membered heteroaryl, and are substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R^{20a}, R^{20b}, R^{20c} and R^{20d} groups are covalently bonded together and, together with the intermediate atoms, comprise a 5- to 6-membered heteroaryl, and are monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 5- to 6-membered heteroaryl, and are unsubstituted.

In a further aspect, two adjacent R^{20a}, R^{20b}, R^{20c}, and R^{20d} groups are covalently bonded together and, together with the intermediate atoms, comprise a 5-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 5-membered heteroaryl, and are substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R^{20a}, R^{20b}, R^{20c}, and R^{20d} groups are covalently bonded together and, together with the intermediate atoms, comprise a 5-membered heteroaryl, and are substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R^{20a}, R^{20b}, R^{20c}, and R^{20d} groups are covalently bonded together and, together with the intermediate atoms, comprise a 5-membered heteroaryl, and are monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 5-membered heteroaryl, and are unsubstituted.

In a further aspect, two adjacent R^{20a}, R^{20b}, R^{20c}, and R^{20d} groups are covalently bonded together and, together with the intermediate atoms, comprise a 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 6-membered heteroaryl, and are substituted with 0, 1, or 2 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In yet a further aspect, two adjacent R^{20a}, R^{20b}, R^{20c}, and R^{20d} groups are covalently bonded together and, together with the intermediate atoms, comprise a 6-membered heteroaryl, and are substituted with 0 or 1 group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In an even further aspect, two adjacent R^{20a}, R^{20b}, R^{20c}, and R^{20d} groups are covalently bonded together and, together with the intermediate atoms, comprise a 6-membered heteroaryl, and are monosubstituted with a group selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl). In a still further aspect, two adjacent R^{20a}, R^{20b}, R^{20c}, and R^{20d} groups are covalently bonded together and, together with the intermediate atoms, comprise a 6-membered heteroaryl, and are unsubstituted.

In a further aspect, R^{20a} is halogen. In a still further aspect, R^{20a} is —Cl.

i. R^{21a}, R^{21b}, R^{21c}, and R^{21d} Groups

In one aspect, each of R^{21a}, R^{21b}, R^{21c}, and R^{21d} is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a further aspect, each of R^{21a}, R^{21b}, R^{21c}, and R^{21d} is hydrogen.

In a further aspect, each of R^{21a}, R^{21b}, R^{21c}, and R^{21d} is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{21a}, R^{21b}, R^{21c}, and R^{21d} is independently selected from hydrogen, —F, —Cl, —NH₂, —CN, —OH, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{21a}, R^{21b}, R^{21c}, and R^{21d} is independently from hydrogen, —F, —Cl, —NH₂, —CN, —OH, methyl, —CH₂F, —CH₂Cl, —CH₂CN, —CH₂OH, —OCH₃, —NHCH₃, and —N(CH₃)₂.

In a further aspect, each of R^{21a}, R^{21b}, R^{21c}, and R^{21d} is independently selected from hydrogen, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R^{21a}, R^{21b}, R^{21c}, and R^{21d} is independently selected from hydrogen, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In yet a further aspect, each of R^{21a}, R^{21b}, R^{21c}, and R^{21d} is independently selected from hydrogen, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In an even further aspect, each of R^{21a}, R^{21b}, R^{21c}, and R^{21d} is independently selected from hydrogen, methyl, —CH₂F, —CH₂Cl, —CH₂CN, —CH₂OH, —OCH₃, —NHCH₃, and —N(CH₃)₂.

In a further aspect, each of R^{21a}, R^{21b}, R^{21c}, and R^{21d} is independently selected from hydrogen, —F, —Cl, —NH₂, —CN, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R^{21a}, R^{21b}, R^{21c}, and R^{21d} is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{21a}, R^{21b}, R^{21c}, and R^{21d} is independently selected from hydrogen, —F, —Cl, —NH₂, —CN, —OH, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{21a}, R^{21b}, R^{21c}, and R^{21d} is independently selected from hydrogen, —F, —Cl, —NH₂, —CN, —OH, methyl, —CH₂F, —CH₂Cl, —CH₂CN, —CH₂OH, —OCH₃, —NHCH₃, and —N(CH₃)₂.

In a further aspect, each of R^{21a}, R^{21b}, R^{21c}, and R^{21d} is independently selected from hydrogen, —F, —Cl, —NH₂, —CN, —OH, C1-C4 alkyl, and C1-C4 alkoxy. In a still further aspect, each of R^{21a}, R^{21b}, R^{21c}, and R^{21d} is independently selected from hydrogen, —F, —Cl, —NH₂, —CN, —OH, methyl, ethyl, n-propyl, i-propyl, —OCH₃, —OCH₂CH₃, —OCH₂CH₂CH₃, and —OCH(CH₃)CH₃. In a still further aspect, each of R^{21a}, R^{21b}, R^{21c}, and R^{21d} is independently selected from hydrogen, —F, —Cl, —NH₂, —CN, —OH, methyl, ethyl, —OCH₃, and —OCH₂CH₃. In yet a further aspect, each of R^{21a}, R^{21b}, R^{21c}, and R^{21d} is independently selected from hydrogen, —F, —Cl, —NH₂, —CN, —OH, methyl, and —OCH₃.

In a further aspect, each of R^{21a}, R^{21b}, R^{21c}, and R^{21d} is independently selected from hydrogen, C1-C4 alkyl, C1-C4 haloalkyl, and C1-C4 alkoxy. In a still further aspect, each of R^{21a}, R^{21b}, R^{21c}, and R^{21d} is independently selected from hydrogen, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, and —OCH(CH₃)CH₃. In yet a further aspect, each of R^{21a}, R^{21b}, R^{21c}, and R^{21d} is independently selected from hydrogen, methyl, ethyl, —CH₂F, —CH₂Cl, —CH₂CH₂F, —CH₂CH₂Cl, —OCH₃, and —OCH₂CH₃. In yet a further aspect, each of R^{21a}, R^{2lb}, R^{21c}, and R^{2ld} is independently selected from hydrogen, methyl, —CH₂F, —CH₂Cl, and —OCH₃.

In a further aspect, each of R^{21a}, R^{21b}, R^{21c}, and R^{21d} is independently selected from hydrogen and C1-C4 haloalkyl. In a still further aspect, each of R^{21a}, R^{2lb}, R^{21c}, and R^{21d} is independently selected from hydrogen, —CH₂F, —CH₂Cl, —CH₂CH₂F, —CH₂CH₂Cl, —CH₂CH₂CH₂F, —CH₂CH₂CH₂Cl, —CH(CH₃)CH₂F, and —CH(CH₃)CH₂Cl. In yet a further aspect, each of R^{21a}, R^{21b}, R^{21c}, and R^{21d} is independently selected from hydrogen, —CH₂F, —CH₂Cl, —CH₂CH₂F, and —CH₂CH₂Cl. In yet a further aspect, each of R^{21a}, R^{21b}, R^{21c}, and R^{21d} is independently selected from hydrogen, —CH₂F and —CH₂Cl.

In a further aspect, each of R^{21a}, R^{21b}, R^{21c}, and R^{21d} is independently selected from hydrogen and C1-C4 alkyl. In a still further aspect, each of R^{21a}, R^{21b}, R^{21c}, and R^{21d} is independently selected from hydrogen, methyl, ethyl, n-propyl, i-propyl, n-butyl, i-butyl, s-butyl, and t-butyl. In yet a further aspect, each of R^{21a}, R^{21b}, R^{21c}, and R^{21d} is independently selected from hydrogen, methyl, ethyl, n-propyl, and i-propyl. In an even further aspect, each of R^{21a}, R^{21b}, R^{21c}, and R^{21d} is independently selected from hydrogen, methyl and ethyl. In a still further aspect, each of R^{21a}, R^{21b}, R^{21c}, and R^{21d} is independently selected from hydrogen and ethyl. In yet a further aspect, each of R^{21a}, R^{21b}, R^{21c}, and R^{21d} is independently selected from hydrogen and methyl.

In a further aspect, each of R^{21a}, R^{21b}, R^{21c}, and R^{21d} is independently selected from hydrogen, halogen, —NH₂, —CN, —OH, C1-C4 alkyl, C1-C4 alkoxy, C1-C4 alkylamino, and (C1-C4)(C1-C4) dialkylamino. In a further aspect, each of R^{21a}, R^{21b}, R^{21c}, and R^{21d} is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{21a}, R^{21b}, R^{21c}, and R^{21d} is independently selected from hydrogen, —F, —Cl, —NH₂, —CN, —OH, methyl, ethyl, —CH₂F, —CH₂Cl, —CH₂CH₂F, —CH₂CH₂Cl, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{21a}, R^{21b}, R^{21c}, and R^{21d} is independently selected from hydrogen, —F, —Cl, —NH₂, —CN, —OH, methyl, —CH₂F, —CH₂Cl, —OCH₃, —NHCH₃, and —N(CH₃)₂.

In a further aspect, each of R^{21a}, R^{21b}, R^{21c}, and R^{21d} is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{21a}, R^{21b}, R^{21c}, and R^{21d} is independently selected from hydrogen, —F, —Cl, —NH₂, —CN, —OH, methyl, ethyl, —CH₂F, —CH₂Cl, —CH₂CH₂F, —CH₂CH₂Cl, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{21a}, R^{21b}, R^{21c}, and R^{21d} is independently selected from hydrogen, —F, —Cl, —NH₂, —CN, —OH, methyl, —CH₂F, —CH₂Cl, —OCH₃, —NHCH₃, and —N(CH₃)₂.

j. R^{22a}, R^{22b}, R^{22c}, R^{22d}, R^{22e}, R^{22f}, R^{22g}, and R^{22h} Groups

In one aspect, each of R^{22a}, R^{22b}, R^{22c}, R^{22d}, R^{22e}, R^{22f}, R^{22g}, and R^{22f} is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a further aspect, each of R^{21a}, R^{21b}, R^{21c}, and R^{21d} is hydrogen.

In a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{22a}, R^{22b}, R^{22c}, R^{22d}, R^{22e}, R^{22f}, R^{22g}, and R^{22f} is independently from hydrogen, —F, —Cl, —NH₂, —CN, —OH, methyl, —CH₂F, —CH₂Cl, —CH₂CN, —CH₂OH, —OCH₃, —NHCH₃, and —N(CH₃)₂.

In a further aspect, each of R^{22a}, R^{22b}, R^{22c}, R^{22d}, R^{22e}, R^{22f}, R^{22g}, and R^{22f} is independently selected from hydrogen, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R^{22a}, R^{22b}, R^{22c}, R^{22d}, R^{22e}, R^{22f}, R^{22g}, and R^{22f} is independently selected from hydrogen, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In yet a further aspect, each of R^{22a}, R^{22b}, R^{22c}, R^{22d}, R^{22e}, R^{22f}, R^{22g}, and R^{22f} is independently selected from hydrogen, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In an even further aspect, each of R^{22a}, R^{22b}, R^{22c}, R^{22d}, R^{22e}, R^{22f}, R^{22g}, and R^{22f} is independently selected from hydrogen, methyl, —CH₂F, —CH₂Cl, —CH₂CN, —CH₂OH, —OCH₃, —NHCH₃, and —N(CH₃)₂.

In a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, —CH₂F, —CH₂Cl, —CH₂CN, —CH₂OH, —OCH₃, —NHCH₃, and —N(CH₃)₂.

In a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, C1-C4 alkyl, and C1-C4 alkoxy. In a still further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, n-propyl, i-propyl, —OCH₃, —OCH₂CH₃, —OCH₂CH₂CH₃, and —OCH(CH₃)CH₃. In a still further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, —OCH₃, and —OCH₂CH₃. In yet a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, and —OCH₃.

In a further aspect, each of R^{22a}, R^{22b}, R^{22c}, R^{22d}, R^{22e}, R^{22f}, R^{22g}, and R^{22f} is independently selected from hydrogen, C1-C4 alkyl, C1-C4 haloalkyl, and C1-C4 alkoxy. In a still further aspect, each of R^{22a}, R^{22b}, R^{22c}, R^{22d}, R^{22e}, R^{22f}, R^{22g}, and R^{22f} is independently selected from hydrogen, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, and —OCH(CH₃)CH₃. In yet a further aspect, each of R^{22a}, R^{22b}, R^{22c}, R^{22d}, R^{22e}, R^{22f}, R^{22g}, and R^{22f} is independently selected from hydrogen, methyl, ethyl, —CH₂F, —CH₂Cl, —CH₂CH₂F, —CH₂CH₂Cl, —OCH₃, and —OCH₂CH₃. In yet a further aspect, each of R^{22a}, R^{22b}, R^{22c}, R^{22d}, R^{22e}, R^{22f}, R^{22g}, and R^{22f} is independently selected from hydrogen, methyl, —CH₂F, —CH₂Cl, and —OCH₃.

In a further aspect, each of R^{22a}, R^{22b}, R^{22c}, R^{22d}, R^{22e}, R^{22f}, R^{22g}, and R^{22f} is independently selected from hydrogen and C1-C4 haloalkyl. In a still further aspect, each of R^{22a}, R^{22b}, R^{22c}, R^{22d}, R^{22e}, R^{22f}, R^{22g}, and R^{22f} is independently selected from hydrogen, —CH₂F, —CH₂Cl, —CH₂CH₂F, —CH₂CH₂Cl, —CH₂CH₂CH₂F, —CH₂CH₂CH₂Cl, —CH(CH₃)CH₂F, and —CH(CH₃)CH₂Cl. In yet a further aspect, each of R^{22a}, R^{22b}, R^{22c}, R^{22d}, R^{22e}, R^{22f}, R^{22g}, and R^{22f} is independently selected from hydrogen, —CH₂F, —CH₂Cl, —CH₂CH₂F, and —CH₂CH₂Cl. In yet a further aspect, each of R^{22a}, R^{22b}, R^{22c}, R^{22d}, R^{22e}, R^{22f}, R^{22g}, and R^{22f} is independently selected from hydrogen, —CH₂F and —CH₂Cl.

In a further aspect, each of R^{22a}, R^{22b}, R^{22c}, R^{22d}, R^{22e}, R^{22f}, R^{22g}, and R^{22f} is independently selected from hydrogen and C1-C4 alkyl. In a still further aspect, each of R^{22a}, R^{22b}, R^{22c}, R^{22d}, R^{22e}, R^{22f}, R^{22g}, and R^{22f} is independently selected from hydrogen, methyl, ethyl, n-propyl, i-propyl, n-butyl, i-butyl, s-butyl, and t-butyl. In yet a further aspect, each of R^{22a}, R^{22b}, R^{22c}, R^{22d}, R^{22e}, R^{22f}, R^{22g}, and R^{22f} is independently selected from hydrogen, methyl, ethyl, n-propyl, and i-propyl. In an even further aspect, each of R^{22a}, R^{22b}, R^{22c}, R^{22d}, R^{22e}, R^{22f}, R^{22g}, and R^{22f} is independently selected from hydrogen, methyl and ethyl. In a still further aspect, each of R^{22a}, R^{22b}, R^{22c}, R^{22d}, R^{22e}, R^{22f}, R^{22g}, and R^{22f} is independently selected from hydrogen and ethyl. In yet a further aspect, each of R^{22a}, R^{22b}, R^{22c}, R^{22d}, R^{22e}, R^{22f}, R^{22g}, and R^{22f} is independently selected from hydrogen and methyl.

In a further aspect, each of R^{22a}, R^{22b}, R^{22c}, R^{22d}, R^{22e}, R^{22f}, R^{22g}, and R^{22f} is independently selected from hydrogen, halogen, —NH₂, —CN, —OH, C1-C4 alkyl, C1-C4 alkoxy, C1-C4 alkylamino, and (C1-C4)(C1-C4) dialkylamino. In a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, —CH₂F, —CH₂Cl, —CH₂CH₂F, —CH₂CH₂Cl, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, —CH₂F, —CH₂Cl, —OCH₃, —NHCH₃, and —N(CH₃)₂.

In a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, ethyl, —CH₂F, —CH₂Cl, —CH₂CH₂F, —CH₂CH₂Cl, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{22a}, R^{22b}, R^{22c}, R^{22d}, R^{22e}, R^{22f}, R^{22g}, and R^{22f} is independently selected from hydrogen, —F, —Cl, —NH₂, —CN, —OH, methyl, —CH₂F, —CH₂Cl, —OCH₃, —NHCH₃, and —N(CH₃)₂.

k. R^{30a} and R^{30b} Groups

In one aspect, each of R^{30a} and R^{30b}, when present, is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a further aspect, each of R^{30a} and R^{30b}, when present, is hydrogen.

In a further aspect, each of R^{30a} and R^{30b}, when present, is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{30a} and R^{30b}, when present, is independently selected from hydrogen, —F, —Cl, —NH₂, —CN, —OH, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{30a} and R^{30b}, when present, is independently from hydrogen, —F, —Cl, —NH₂, —CN, —OH, methyl, —CH₂F, —CH₂Cl, —CH₂CN, —CH₂OH, —OCH₃, —NHCH₃, and —N(CH₃)₂.

In a further aspect, each of R^{30a} and R^{30b}, when present, is independently selected from hydrogen, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R^{30a} and R^{30b}, when present, is independently selected from hydrogen, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In yet a further aspect, each of R^{30a} and R^{30b}, when present, is independently selected from hydrogen, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In an even further aspect, each of R^{30a} and R^{30b}, when present, is independently selected from hydrogen, methyl, —CH₂F, —CH₂Cl, —CH₂CN, —CH₂OH, —OCH₃, —NHCH₃, and —N(CH₃)₂.

In a further aspect, each of R^{30a} and R^{30b}, when present, is independently selected from hydrogen, —F, —Cl, —NH₂, —CN, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, each of R^{30a} and R^{30b}, when present, is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{30a} and R^{30b}, when present, is independently selected from hydrogen, —F, —Cl, —NH₂, —CN, —OH, methyl, ethyl, —CH₂F, —CH₂Cl, —CH₂CH₂F, —CH₂CH₂Cl, —CH(CH₃)CH₂Cl, —CH₂CN, —CH₂CH₂CN, —CH₂OH, —CH₂CH₂OH, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{30a} and R^{30b}, when present, is independently selected from hydrogen, —F, —Cl, —NH₂, —CN, —OH, methyl, —CH₂F, —CH₂Cl, —CH₂CN, —CH₂OH, —OCH₃, —NHCH₃, and —N(CH₃)₂.

In a further aspect, each of R^{30a} and R^{30b}, when present, is independently selected from hydrogen, —F, —Cl, —NH₂, —CN, —OH, C1-C4 alkyl, and C1-C4 alkoxy. In a still further aspect, each of R^{30a} and R^{30b}, when present, is independently selected from hydrogen, —F, —Cl, —NH₂, —CN, —OH, methyl, ethyl, n-propyl, i-propyl, —OCH₃, —OCH₂CH₃, —OCH₂CH₂CH₃, and —OCH(CH₃)CH₃. In a still further aspect, each of R^{30a} and R^{30b}, when present, is independently selected from hydrogen, —F, —Cl, —NH₂, —CN, —OH, methyl, ethyl, —OCH₃, and —OCH₂CH₃. In yet a further aspect, each of R^{30a} and R^{30b}, when present, is independently selected from hydrogen, —F, —Cl, —NH₂, —CN, —OH, methyl, and —OCH₃.

In a further aspect, each of R^{30a} and R^{30b}, when present, is independently selected from hydrogen, C1-C4 alkyl, C1-C4 haloalkyl, and C1-C4 alkoxy. In a still further aspect, each of R^{30a} and R^{30b}, when present, is independently selected from hydrogen, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, and —OCH(CH₃)CH₃. In yet a further aspect, each of R^{30a} and R^{30b}, when present, is independently selected from hydrogen, methyl, ethyl, —CH₂F, —CH₂Cl, —CH₂CH₂F, —CH₂CH₂Cl, —OCH₃, and —OCH₂CH₃. In yet a further aspect, each of R^{30a} and R^{30b}, when present, is independently selected from hydrogen, methyl, —CH₂F, —CH₂Cl, and —OCH₃.

In a further aspect, each of R^{30a} and R^{30b}, when present, is independently selected from hydrogen and C1-C4 haloalkyl. In a still further aspect, each of R^{30a} and R^{30b}, when present, is independently selected from hydrogen, —CH₂F, —CH₂Cl, —CH₂CH₂F, —CH₂CH₂Cl, —CH₂CH₂CH₂F, —CH₂CH₂CH₂Cl, —CH(CH₃)CH₂F, and —CH(CH₃)CH₂Cl. In yet a further aspect, each of R^{30a} and R^{30b}, when present, is independently selected from hydrogen, —CH₂F, —CH₂Cl, —CH₂CH₂F, and —CH₂CH₂Cl. In yet a further aspect, each of R^{30a} and R^{30b}, when present, is independently selected from hydrogen, —CH₂F and —CH₂Cl.

In a further aspect, each of R^{30a} and R^{30b}, when present, is independently selected from hydrogen and C1-C4 alkyl. In a still further aspect, each of R^{30a} and R^{30b}, when present, is independently selected from hydrogen, methyl, ethyl, n-propyl, i-propyl, n-butyl, i-butyl, s-butyl, and t-butyl. In yet a further aspect, each of R^{30a} and R^{30b}, when present, is independently selected from hydrogen, methyl, ethyl, n-propyl, and i-propyl. In an even further aspect, each of R^{30a} and R^{30b}, when present, is independently selected from hydrogen, methyl and ethyl. In a still further aspect, each of R^{30a} and R^{30b}, when present, is independently selected from hydrogen and ethyl. In yet a further aspect, each of R^{30a} and R^{30b}, when present, is independently selected from hydrogen and methyl.

In a further aspect, each of R^{30a} and R^{30b}, when present, is independently selected from hydrogen, halogen, —NH₂, —CN, —OH, C1-C4 alkyl, C1-C4 alkoxy, C1-C4 alkylamino, and (C1-C4)(C1-C4) dialkylamino. In a further aspect, each of R^{30a} and R^{30b}, when present, is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{30a} and R^{30b}, when present, is independently selected from hydrogen, —F, —Cl, —NH₂, —CN, —OH, methyl, ethyl, —CH₂F, —CH₂Cl, —CH₂CH₂F, —CH₂CH₂Cl, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{30a} and R^{30b}, when present, is independently selected from hydrogen, —F, —Cl, —NH₂, —CN, —OH, methyl, —CH₂F, —CH₂Cl, —OCH₃, —NHCH₃, and —N(CH₃)₂.

In a further aspect, each of R^{30a} and R^{30b}, when present, is independently selected from hydrogen, —F, —Cl, —NH₂, —CN, —OH, methyl, ethyl, n-propyl, i-propyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, and —N(CH₃)(CH₂CH₃). In a still further aspect, each of R^{30a} and R^{30b}, when present, is independently selected from hydrogen, —F, —Cl, —NH₂, —CN, —OH, methyl, ethyl, —CH₂F, —CH₂Cl, —CH₂CH₂F, —CH₂CH₂Cl, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, each of R^{30a} and R^{30b}, when present, is independently selected from hydrogen, —F, —Cl, —NH₂, —CN, —OH, methyl, —CH₂F, —CH₂Cl, —OCH₃, —NHCH₃, and —N(CH₃)₂.

l. Ar¹ Groups

In one aspect, Ar¹ is selected from 6-membered monocyclic aryl and pyridinyl, and is substituted with 0, 1, 2, 3, or 4 R² groups. In a further aspect, Ar¹ is selected from 6-membered monocyclic aryl and pyridinyl, and is substituted with 0, 1, 2, or 3 R² groups. In a still further aspect, Ar¹ is selected from 6-membered monocyclic aryl and pyridinyl, and is substituted with 0, 1, or 2 R² groups. In yet a further aspect, Ar¹ is selected from 6-membered monocyclic aryl and pyridinyl, and is substituted with 0 or 1 R² groups. In an even further aspect, Ar¹ is selected from 6-membered monocyclic aryl and pyridinyl, and is monosubstituted with a R² groups. In a still further aspect, Ar¹ is selected from 6-membered monocyclic aryl and pyridinyl, and is substituted with 0 R² groups.

In a further aspect, Ar¹ is 6-membered monocyclic aryl substituted with 0, 1, 2, 3, or 4 R² groups. In a still further aspect, Ar¹ is 6-membered monocyclic aryl substituted with 0, 1, 2, or 3 R² groups. In yet a further aspect, Ar¹ is 6-membered monocyclic aryl substituted with 0, 1, or 2 R² groups. In an even further aspect, Ar¹ is 6-membered monocyclic aryl substituted with 0 or 1 R² groups. In a still further aspect, Ar¹ is 6-membered monocyclic aryl monosubstituted with a R² group. In yet a further aspect, Ar¹ is 6-membered monocyclic aryl substituted with 0 R² groups.

In a further aspect, Ar¹ is pyridinyl substituted with 0, 1, 2, 3, or 4 R² groups. In a still further aspect, Ar¹ is pyridinyl substituted with 0, 1, 2, or 3 R² groups. In yet a further aspect, Ar¹ is pyridinyl substituted with 0, 1, or 2 R² groups. In an even further aspect, Ar¹ is pyridinyl substituted with 0 or 1 R² groups. In a still further aspect, Ar¹ is pyridinyl monosubstituted with a R² group. In yet a further aspect, Ar¹ is pyridinyl substituted with 0 R² groups.

In a further aspect, Ar¹ is selected from 6-membered monocyclic aryl and pyridinyl, and is substituted with 1 halogen group. In a still further aspect, Ar¹ is 6-membered monocyclic aryl substituted with 1 halogen group. In yet a further aspect, Ar¹ is pyridinyl substituted with 1 halogen group.

In a further aspect, Ar¹ is selected from 6-membered monocyclic aryl and pyridinyl, and is substituted with 1 —Cl group. In a still further aspect, Ar¹ is 6-membered monocyclic aryl substituted with 1 —Cl group. In yet a further aspect, Ar¹ is pyridinyl substituted with 1 —Cl group.

m. Cy¹ Groups

In one aspect, Cy¹, when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a further aspect, Cy¹, when present, is selected from cycloalkyl, heterocycloalkyl, aryl, and heteroaryl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is selected from cycloalkyl, heterocycloalkyl, aryl, and heteroaryl, and is substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is selected from cycloalkyl, heterocycloalkyl, aryl, and heteroaryl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from cycloalkyl, heterocycloalkyl, aryl, and heteroaryl, and is unsubstituted.

In a further aspect, Cy¹, when present, is selected from cycloalkyl and heterocycloalkyl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from cycloalkyl and heterocycloalkyl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is selected from cycloalkyl and heterocycloalkyl, and is substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is selected from cycloalkyl and heterocycloalkyl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from cycloalkyl and heterocycloalkyl, and is unsubstituted.

In a further aspect, Cy¹, when present, is cycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is cycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is cycloalkyl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is cycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is unsubstituted cycloalkyl.

In a further aspect, Cy¹, when present, is selected from cyclopropyl, cyclobutyl, cyclopentyl, and cyclohexyl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from cyclopropyl, cyclobutyl, cyclopentyl, and cyclohexyl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is selected from cyclopropyl, cyclobutyl, cyclopentyl, and cyclohexyl, and is substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is selected from cyclopropyl, cyclobutyl, cyclopentyl, and cyclohexyl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from cyclopropyl, cyclobutyl, cyclopentyl, and cyclohexyl, and is unsubstituted.

In a further aspect, Cy¹, when present, is heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is heterocycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is heterocycloalkyl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is heterocycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is unsubstituted heterocycloalkyl.

In a further aspect, Cy¹, when present, is selected from azetidinyl, aziridinyl, oxetanyl, oxiranyl, pyrrolidonyl, tetrahydrofuranyl, tetrahydrothiophenyl, thietanyl, and thiiranyl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from azetidinyl, aziridinyl, oxetanyl, oxiranyl, pyrrolidonyl, tetrahydrofuranyl, tetrahydrothiophenyl, thietanyl, and thiiranyl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is selected from azetidinyl, aziridinyl, oxetanyl, oxiranyl, pyrrolidonyl, tetrahydrofuranyl, tetrahydrothiophenyl, thietanyl, and thiiranyl, and is substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is selected from azetidinyl, aziridinyl, oxetanyl, oxiranyl, pyrrolidonyl, tetrahydrofuranyl, tetrahydrothiophenyl, thietanyl, and thiiranyl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from azetidinyl, aziridinyl, oxetanyl, oxiranyl, pyrrolidonyl, tetrahydrofuranyl, tetrahydrothiophenyl, thietanyl, and thiiranyl, and is unsubstituted.

In a further aspect, Cy¹, when present, is selected from aryl and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from aryl and heteroaryl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is selected from aryl and heteroaryl, and is substituted with 0 or 1 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is selected from aryl and heteroaryl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from aryl and heteroaryl, and is unsubstituted.

In a further aspect, Cy¹, when present, is aryl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is aryl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is aryl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is aryl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is unsubstituted aryl.

In a further aspect, Cy¹, when present, is phenyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is phenyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is phenyl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is phenyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is unsubstituted phenyl.

In a further aspect, Cy¹, when present, is heteroaryl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is heteroaryl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is heteroaryl substituted with 0 or 1 group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is heteroaryl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is unsubstituted heteroaryl.

In a further aspect, Cy¹, when present, is selected from imidazolyl, pyrrolyl, furanyl, isothiazolyl, isoxazolyl, oxazolyl, pyridinyl, pyrimidinyl, thiazolyl, and thiophenyl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from imidazolyl, pyrrolyl, furanyl, isothiazolyl, isoxazolyl, oxazolyl, pyridinyl, pyrimidinyl, thiazolyl, and thiophenyl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy¹, when present, is selected from imidazolyl, pyrrolyl, furanyl, isothiazolyl, isoxazolyl, oxazolyl, pyridinyl, pyrimidinyl, thiazolyl, and thiophenyl, and is substituted with 0 or 1 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy¹, when present, is selected from imidazolyl, pyrrolyl, furanyl, isothiazolyl, isoxazolyl, oxazolyl, pyridinyl, pyrimidinyl, thiazolyl, and thiophenyl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy¹, when present, is selected from imidazolyl, pyrrolyl, furanyl, isothiazolyl, isoxazolyl, oxazolyl, pyridinyl, pyrimidinyl, thiazolyl, and thiophenyl, and is unsubstituted.

n. Cy² Groups

In one aspect, Cy², when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a further aspect, Cy², when present, is selected from cycloalkyl, heterocycloalkyl, aryl, and heteroaryl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from cycloalkyl, heterocycloalkyl, aryl, and heteroaryl, and is substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is selected from cycloalkyl, heterocycloalkyl, aryl, and heteroaryl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is selected from cycloalkyl, heterocycloalkyl, aryl, and heteroaryl, and is unsubstituted.

In a further aspect, Cy², when present, is selected from cycloalkyl and heterocycloalkyl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from cycloalkyl and heterocycloalkyl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is selected from cycloalkyl and heterocycloalkyl, and is substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is selected from cycloalkyl and heterocycloalkyl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from cycloalkyl and heterocycloalkyl, and is unsubstituted.

In a further aspect, Cy², when present, is cycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is cycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is cycloalkyl substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is cycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is cycloalkyl unsubstituted.

In a further aspect, Cy², when present, is selected from cyclopentyl and cyclohexyl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from cyclopentyl and cyclohexyl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is selected from cyclopentyl and cyclohexyl, and is substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is selected from cyclopentyl and cyclohexyl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from cyclopentyl and cyclohexyl, and is unsubstituted.

In a further aspect, Cy², when present, is heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is heterocycloalkyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is heterocycloalkyl substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is heterocycloalkyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is heterocycloalkyl unsubstituted.

In a further aspect, Cy², when present, is selected from morpholinyl, piperidinyl, and pyrrolidinyl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from morpholinyl, piperidinyl, and pyrrolidinyl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is selected from morpholinyl, piperidinyl, and pyrrolidinyl, and is substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is selected from morpholinyl, piperidinyl, and pyrrolidinyl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from morpholinyl, piperidinyl, and pyrrolidinyl, and is unsubstituted.

In a further aspect, Cy², when present, is selected from aryl and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from aryl and heteroaryl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is selected from aryl and heteroaryl, and is substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is selected from aryl and heteroaryl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from aryl and heteroaryl, and is unsubstituted.

In a further aspect, Cy², when present, is aryl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is aryl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is aryl substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is aryl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is aryl unsubstituted.

In a further aspect, Cy², when present, is phenyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is phenyl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is phenyl substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is phenyl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is phenyl unsubstituted. In yet a further aspect, Cy², when present, is phenyl monosubstituted with halogen.

In a further aspect, Cy², when present, is heteroaryl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is heteroaryl substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is heteroaryl substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is heteroaryl monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is heteroaryl unsubstituted.

In a further aspect, Cy², when present, is selected from pyrrolyl, pyridinyl, and primidinyl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from pyrrolyl, pyridinyl, and primidinyl, and is substituted with 0, 1, or 2 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In yet a further aspect, Cy², when present, is selected from pyrrolyl, pyridinyl, and primidinyl, and is substituted with 0 or 1 groups selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In an even further aspect, Cy², when present, is selected from pyrrolyl, pyridinyl, and primidinyl, and is monosubstituted with a group selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino. In a still further aspect, Cy², when present, is selected from pyrrolyl, pyridinyl, and primidinyl, and is unsubstituted.

### 2. Example Compounds

In one aspect, a compound can be present as one or more of the following structures:

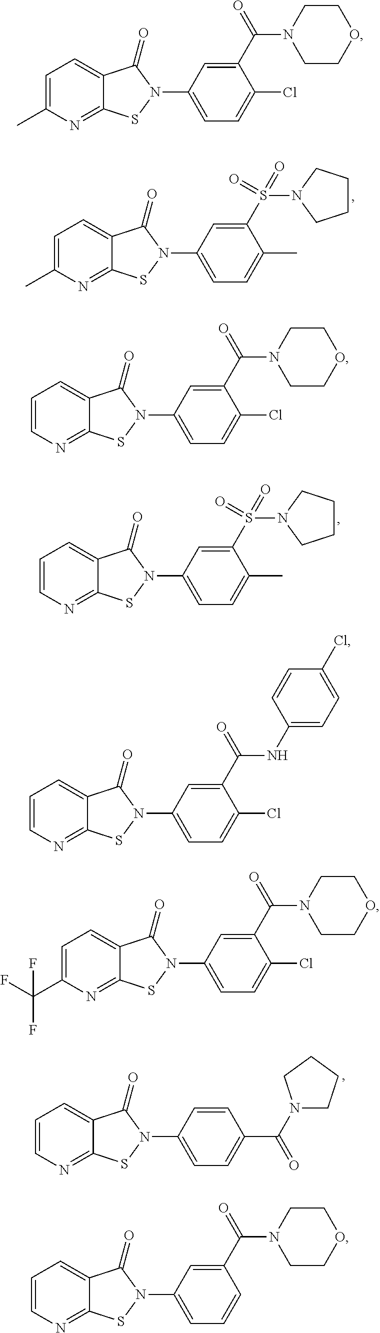

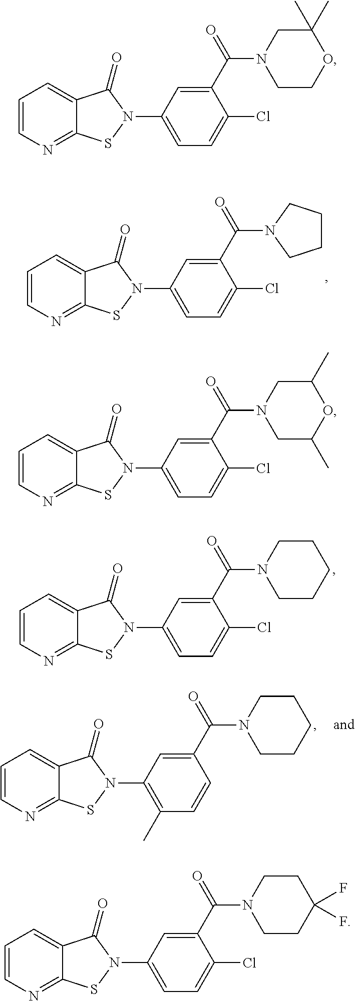

or a pharmaceutically acceptable salt thereof.

### 3. Prophetic Compounds

The following compound examples are prophetic, and can be prepared using the synthesis methods described herein above and other general methods as needed as would be known to one skilled in the art. It is anticipated that the prophetic compounds would be active as inhibitors of a viral infection, and such activity can be determined using the assay methods described herein below.

In one aspect, a compound can be selected from:

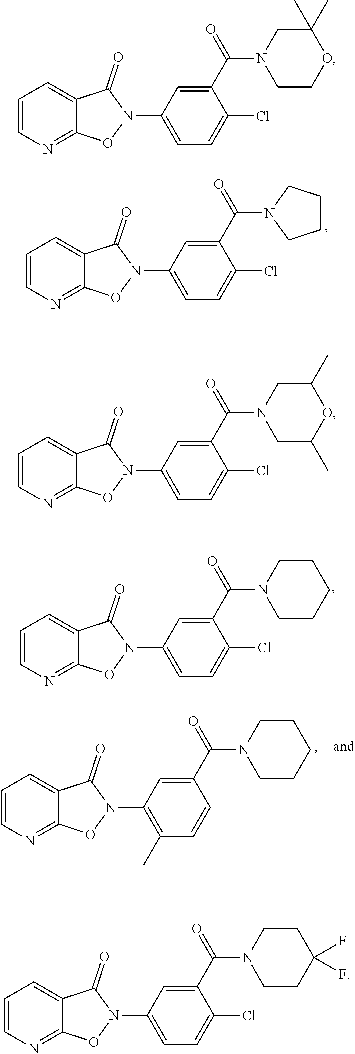

In one aspect, a compound can be selected from:

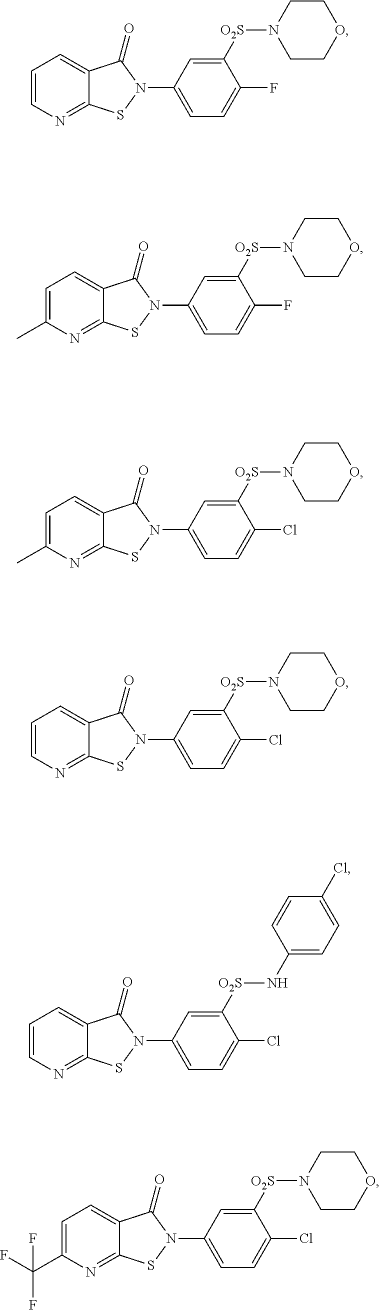 [SMILES: O=c1c2cccnc2sn1-c1ccc(F)c(S(=O)(=O)N2CCOCC2)c1 ; Cc1ccc2c(=O)n(-c3ccc(F)c(S(=O)(=O)N4CCOCC4)c3)sc2n1 ; Cc1ccc2c(=O)n(-c3ccc(Cl)c(S(=O)(=O)N4CCOCC4)c3)sc2n1 ; O=c1c2cccnc2sn1-c1ccc(Cl)c(S(=O)(=O)N2CCOCC2)c1 ; O=c1c2cccnc2sn1-c1ccc(Cl)c(S(=O)(=O)Nc2ccc(Cl)cc2)c1 ; O=c1c2ccc(C(F)(F)F)nc2sn1-c1ccc(Cl)c(S(=O)(=O)N2CCOCC2)c1]

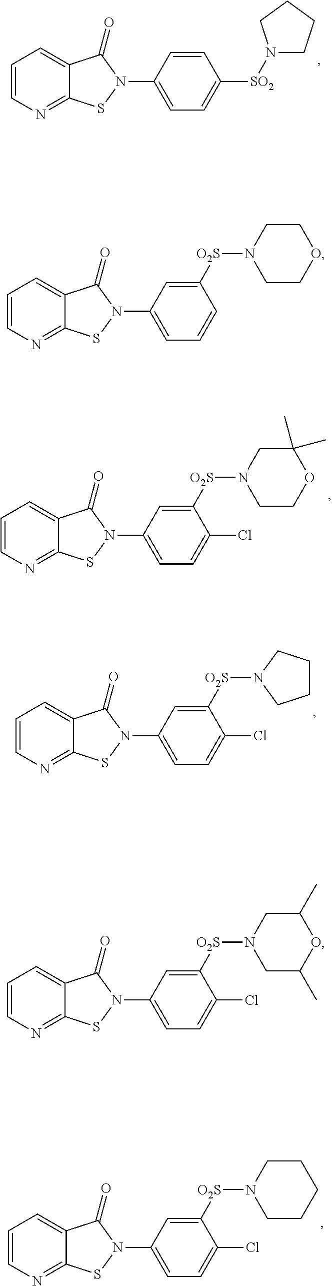 [SMILES: O=c1c2cccnc2sn1-c1ccc(S(=O)(=O)N2CCCC2)cc1 ; O=c1c2cccnc2sn1-c1cccc(S(=O)(=O)N2CCOCC2)c1 ; CC1(C)CN(S(=O)(=O)c2cc(-n3sc4ncccc4c3=O)ccc2Cl)CCO1 ; O=c1c2cccnc2sn1-c1ccc(Cl)c(S(=O)(=O)N2CCCC2)c1 ; CC1CN(S(=O)(=O)c2cc(-n3sc4ncccc4c3=O)ccc2Cl)CC(C)O1 ; O=c1c2cccnc2sn1-c1ccc(Cl)c(S(=O)(=O)N2CCCCC2)c1]

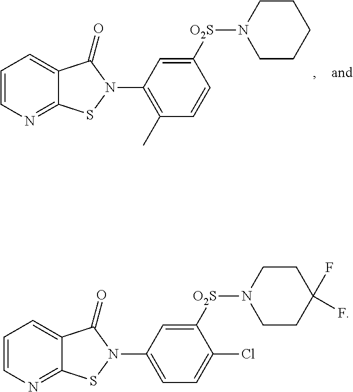 [SMILES: Cc1ccc(S(=O)(=O)N2CCCCC2)cc1-n1sc2ncccc2c1=O ; O=c1c2cccnc2sn1-c1ccc(Cl)c(S(=O)(=O)N2CCC(F)(F)CC2)c1]

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. Methods of Making the Compounds

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-III, as described and exemplified below. The following examples are provided so that the invention might be more fully understood, are illustrative only, and should not be construed as limiting.

1. Route I

In one aspect, intermediates towards the preparation of substituted heterocyclic pyridinones can be prepared as shown below.

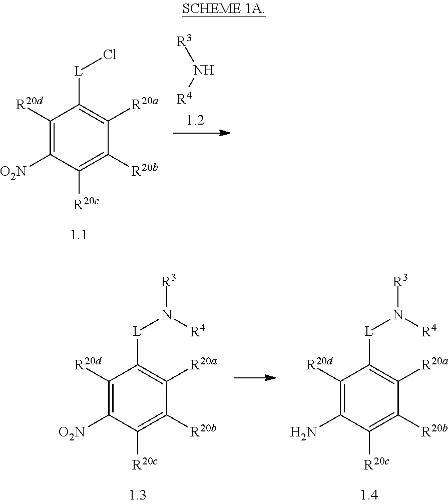

Compounds are represented in generic form, with substituents as noted in compound descriptions elsewhere herein. A more specific example is set forth below.

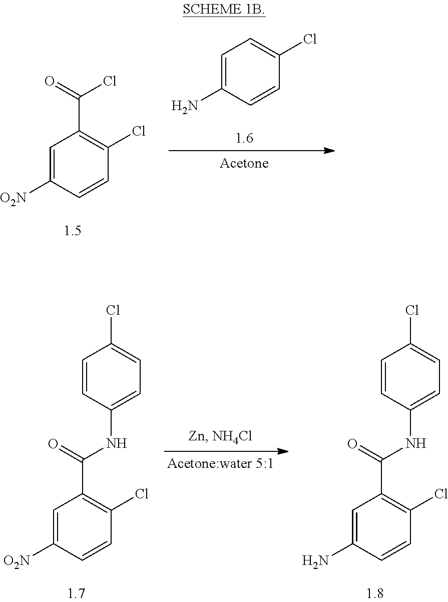 [SMILES: O=C(Cl)c1cc([N+](=O)[O-])ccc1Cl ; Nc1ccc(Cl)cc1 ; O=C(Nc1ccc(Cl)cc1)c1cc([N+](=O)[O-])ccc1Cl ; Nc1ccc(Cl)c(C(=O)Nc2ccc(Cl)cc2)c1]

In one aspect, compounds of type 1.4, and similar compounds, can be prepared according to reaction Scheme 1B above. Thus, compounds of type 1.3 can be prepared by a coupling reaction of an appropriate acid chloride, e.g., 1.1 as shown above, and an appropriate amine, e.g., 1.2. Appropriate acid chlorides and appropriate amines are commercially available or prepared by methods known to one skilled in the art. The coupling reaction is carried out in an appropriate solvent, e.g., acetone. Compounds of type 1.4 can be prepared by reduction of an appropriate nitro analog, e.g., 1.3 as shown above. The reduction is carried out in the presence of an appropriate reducing agent, e.g., zinc metal as shown above, and an appropriate base, e.g., ammonium chloride, in an appropriate solvent system, e.g., acetone: water 5:1. As can be appreciated by one skilled in the art, the above reaction provides an example of a generalized approach wherein compounds similar in structure to the specific reactants above (compounds similar to compounds of type 1.5, 1.6, and 1.7), can be substituted in the reaction to provide intermediates towards the preparation of substituted heterocyclic pyridinone derivatives similar to Formula 1.8.

2. Route II

In one aspect, substituted heterocyclic pyridinones can be prepared as shown below.

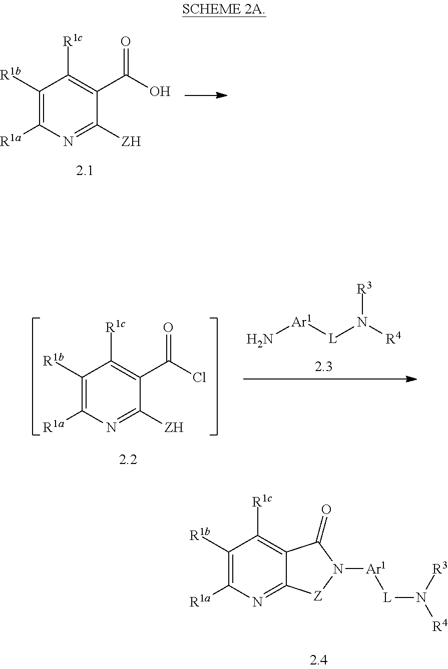

Compounds are represented in generic form, with substituents as noted in compound descriptions elsewhere herein. A more specific example is set forth below.

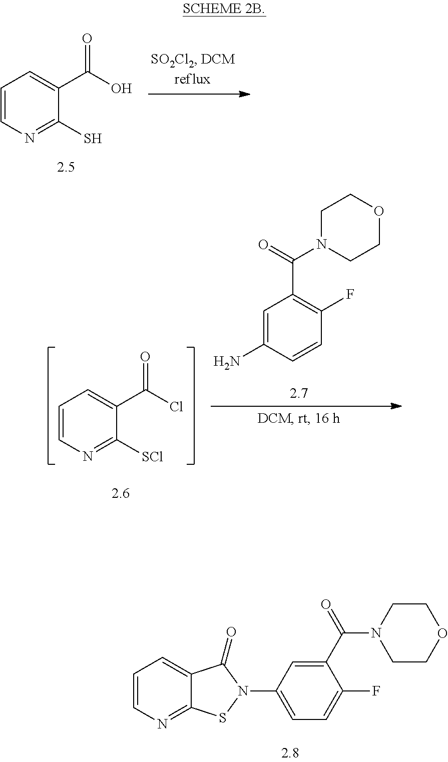

In one aspect, compounds of type 2.4, and similar compounds, can be prepared according to reaction Scheme 2B above. Thus, compounds of type 2.2 can be prepared by a displacement reaction of an appropriate carboxylic acid, e.g., 2.1 as shown above. Appropriate carboxylic acids are commercially available or prepared by methods known to one skilled in the art. The displacement reaction is carried out in the presence of an appropriate electrophile, e.g., thionyl chloride, in an appropriate solvent, e.g., dichloromethane. Compounds of type 2.4 can be prepared by a coupling reaction of an appropriate acid chloride, e.g., 2.2 as shown above, and an appropriate amine, e.g., 2.3 as shown above. Appropriate amines are commercially available or prepared by methods known to one skilled in the art. The coupling reaction is carried out in the presence of an appropriate solvent, e.g., dichloromethane, for an appropriate period of time, e.g., 16 hours. As can be appreciated by one skilled in the art, the above reaction provides an example of a generalized approach wherein compounds similar in structure to the specific reactants above (compounds similar to compounds of type 2.5, 2.6, and 2.7), can be substituted in the reaction to provide substituted heterocyclic pyridinone derivatives similar to Formula 2.8.

3. Route III

In one aspect, substituted heterocyclic pyridinones can be prepared as shown below.

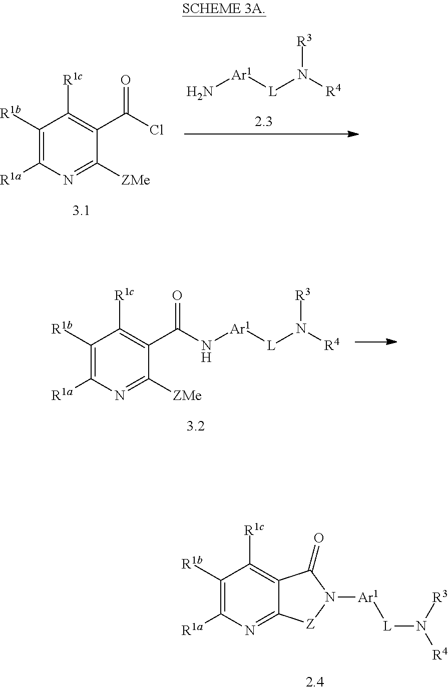

Compounds are represented in generic form, with substituents as noted in compound descriptions elsewhere herein. A more specific example is set forth below.

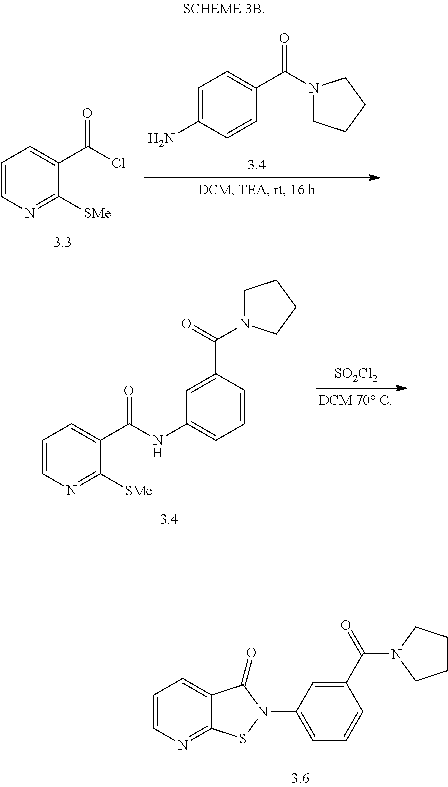

In one aspect, compounds of type 2.4, and similar compounds, can be prepared according to reaction Scheme 3B above. Thus, compounds of type 3.2 can be prepared by a coupling reaction between an appropriate acid chloride, e.g., 3.1 as shown above, and an appropriate amine, e.g., 2.3 as shown above. Appropriate acid chlorides and appropriate amines are commercially available or prepared by methods known to one skilled in the art. The coupling reaction is carried out in the presence of an appropriate base, e.g., triethylamine, in an appropriate solvent, e.g., dichloromethane, for an appropriate period of time, e.g., 16 hours. Compounds of type 2.4 can be prepared by a cyclization reaction of an appropriate amide, e.g., 3.2 as shown above. The cyclization reaction is carried out in the presence of an appropriate electrophilic agent, e.g., thionyl chloride, in an appropriate solvent, e.g., dichloromethane, at an appropriate temperature, e.g., 70° C. As can be appreciated by one skilled in the art, the above reaction provides an example of a generalized approach wherein compounds similar in structure to the specific reactants above (compounds similar to compounds of type 3.3, 3.4, and 3.5), can be substituted in the reaction to provide substituted heterocyclic pyridinone derivatives similar to Formula 3.6.

### D. Pharmaceutical Compositions

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

Thus, in one aspect, disclosed are pharmaceutical compositions comprising a pharmaceutically acceptable carrier and a therapeutically effective amount of at least one compound having a structure represented by a formula:

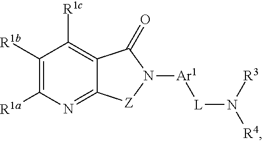

wherein Z is selected from O and S; wherein L is selected from C═O and SO₂; wherein each of R^{1a}, R^{1b}, and R^{1c} is independently selected from hydrogen, —NH₂, C1-C4 alkyl, C1-C4 haloalkyl, —CO₂H, —CO₂(C1-C4 alkyl), C1-C4 alkylamino, (C1-C4)(C1-C4) dialkylamino, and Cy¹; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; wherein Ar¹ is selected from 6-membered monocyclic aryl and pyridinyl, and is substituted with 0, 1, 2, 3, or 4 R² groups; wherein each occurrence of R², when present, is independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl); or wherein any two adjacent R² groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂(C1-C4 alkyl)(C1-C4 alkyl), —SO₂NH₂, —SO₂NH(C1-C4 alkyl), and —SO₂NH(C1-C4 alkyl)(C1-C4 alkyl); wherein R³ is hydrogen or C1-C4 alkyl; wherein R⁴ is selected from C1-C8 alkyl, Cy², and (C1-C4)Cy²; wherein Cy², when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; or wherein each of R³ and R⁴ are covalently bonded together and, together with the intermediate atoms, comprise a 3- to 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino, or a pharmaceutically acceptable salt thereof.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a viral infection such as, for example, HIV-1.

It is understood that the disclosed compositions can be prepared from the disclosed compounds. It is also understood that the disclosed compositions can be employed in the disclosed methods of using.

### E. Methods of Using the Compounds and Compositions

Also provided is a method of use of a disclosed compound, composition, or medicament. In one aspect, the method of use is directed to the treatment of a disorder. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

The pharmaceutical compositions and methods of the present invention can further comprise other therapeutically active compounds as noted herein which are usually applied in the treatment of the above mentioned pathological conditions.

1. Treatment Methods

The compounds disclosed herein are useful for treating or controlling disorders associated with a viral infection, in particular, HIV-1. Thus, provided is a method comprising administering a therapeutically effective amount of a composition comprising a disclosed compound to a subject. In a further aspect, the method can be a method for treating a viral infection.

a. Treating a Viral Infection

In one aspect, disclosed are methods of treating a viral infection in a subject having the viral infection, the method comprising the step of administering to the subject a therapeutically effective amount of at least one disclosed compound, or a pharmaceutically acceptable salt thereof.

Thus, in one aspect, disclosed are compounds having a structure represented by a formula:

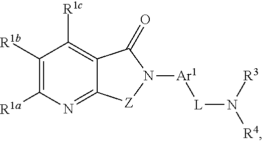

wherein Z is selected from O and S; wherein L is selected from C═O and SO₂; wherein each of R^{1a}, R^{1b}, and R^{1c} is independently selected from hydrogen, —NH₂, C1-C4 alkyl, C1-C4 haloalkyl, —CO₂H, —CO₂(C1-C4 alkyl), C1-C4 alkylamino, (C1-C4)(C1-C4) dialkylamino, and Cy¹; wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; wherein Ar¹ is selected from 6-membered monocyclic aryl and pyridinyl, and is substituted with 0, 1, 2, 3, or 4 R² groups; wherein each occurrence of R², when present, is independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl); or wherein any two adjacent R² groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂(C1-C4 alkyl)(C1-C4 alkyl), —SO₂NH₂, —SO₂NH(C1-C4 alkyl), and —SO₂NH(C1-C4 alkyl)(C1-C4 alkyl); wherein R³ is hydrogen or C1-C4 alkyl; wherein R⁴ is selected from C1-C8 alkyl, Cy², and (C1-C4)Cy²; wherein Cy², when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino; or wherein each of R³ and R⁴ are covalently bonded together and, together with the intermediate atoms, comprise a 3- to 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino, or a pharmaceutically acceptable salt thereof.

Examples of viral infections include, but are not limited to, human immunodeficiency virus (HIV), human papillomavirus (HPV), influenza, chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, chikungunya, Venezuelan equine encephalitis, dengue, influenza, and zika.

In a further aspect, the subject has been diagnosed with a need for treatment of the viral infection prior to the administering step.

In a further aspect, the subject is a mammal. In a still further aspect, the mammal is a human.

In a further aspect, the method further comprises the step of identifying a subject in need of treatment of the viral infection.

In a further aspect, the disorder is associated with a viral infection. In a still further aspect, the viral infection is selected from human immunodeficiency virus (HIV), human papillomavirus (HPV), influenza, chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, chikungunya, Venezuelan equine encephalitis, dengue, influenza, and zika. In yet a further aspect, the viral infection is HIV. In an even further aspect, the HIV is HIV-1.

In a further aspect, the method further comprises the step of administering a therapeutically effective amount of at least one antiviral agent. In a still further aspect, the at least one agent is selected from acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscamet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

In a further aspect, the antiviral agent is a HIV therapeutic agent. In a still further aspect, the HIV therapeutic agent is selected from: a) a HIV fusion/lysis inhibitor, or a pharmaceutically acceptable prodrug, salt, solvate, or polymorph thereof; b) a HIV integrase inhibitor, or a pharmaceutically acceptable prodrug, salt, solvate, or polymorph thereof; c) a HIV non-nucleoside reverse transcriptase inhibitor, or a pharmaceutically acceptable prodrug, salt, solvate, or polymorph thereof; d) a HIV nucleoside reverse transcriptase inhibitor, or a pharmaceutically acceptable prodrug, salt, solvate, or polymorph thereof; and e) a HIV protease inhibitor, or a pharmaceutically acceptable prodrug, salt, solvate, or polymorph thereof.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, and elvitegravir, or a pharmaceutically acceptable prodrug, salt, solvate, or polymorph thereof. In a still further aspect, the HIV integrase inhibitor is raltegravir, or a pharmaceutically acceptable prodrug, salt, solvate, or polymorph thereof. In yet a further aspect, the HIV integrase inhibitor is dolutegravir, or a pharmaceutically acceptable prodrug, salt, solvate, or polymorph thereof. In an even further aspect, the HIV integrase inhibitor is elvitegravir, or a pharmaceutically acceptable prodrug, salt, solvate, or polymorph thereof.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is lersivirine, or a pharmaceutically acceptable prodrug, salt, solvate, or polymorph thereof. In yet a further aspect, the HIV non-nucleoside reverse transcriptase inhibitor is nevirapine, or a pharmaceutically acceptable prodrug, salt, solvate, or polymorph thereof. In an even further aspect, the HIV non-nucleoside reverse transcriptase inhibitor is rilpivirine, or a pharmaceutically acceptable prodrug, salt, solvate, or polymorph thereof.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, elvucitabine, emtricitabine, lamivudine, stavudine, tenofovir, and zidovudine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is elvucitabine, or a pharmaceutically acceptable prodrug, salt, solvate, or polymorph thereof. In yet a further aspect, the HIV nucleoside reverse transcriptase inhibitor is emtricitabine, or a pharmaceutically acceptable prodrug, salt, solvate, or polymorph thereof. In an even further aspect, the HIV nucleoside reverse transcriptase inhibitor is lamivudine, or a pharmaceutically acceptable prodrug, salt, solvate, or polymorph thereof. In a still further aspect, the HIV nucleoside reverse transcriptase inhibitor is stavudine, or a pharmaceutically acceptable prodrug, salt, solvate, or polymorph thereof. In yet a further aspect, the HIV nucleoside reverse transcriptase inhibitor is tenofovir, or a pharmaceutically acceptable prodrug, salt, solvate, or polymorph thereof. In an even further aspect, the HIV nucleoside reverse transcriptase inhibitor is zidovudine, or a pharmaceutically acceptable prodrug, salt, solvate, or polymorph thereof.

In a further aspect, the HIV protease inhibitor is selected from wherein the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof.

In a further aspect, the effective amount is a therapeutically effective amount. In a still further aspect, the effective amount is a prophylactically effective amount.

### 2. Methods of Inhibiting a Viral Infection in a Mammal

In one aspect, disclosed are methods of inhibiting a viral infection in a mammal, the method comprising the step of administering to the mammal a therapeutically effective amount of at least one disclosed compound, or a pharmaceutically acceptable salt thereof.

In a further aspect, the compound exhibits inhibition of a viral infection. In a still further aspect, the compound exhibits a decrease in a viral infection. In yet a further aspect, the viral infection is HIV. In an even further aspect, HIV is HIV-1.

In a further aspect, the compound exhibits inhibition of Nef-Hck activity with an IC₅₀ of less than about 30 μM. In a still further aspect, the compound exhibits inhibition of Nef-Hck activity with an IC₅₀ of less than about 25 μM. In yet a further aspect, the compound exhibits inhibition of Nef-Hck activity with an IC₅₀ of less than about 20 μM. In an even further aspect, the compound exhibits inhibition of Nef-Hck activity with an IC₅₀ of less than about 15 μM. In a still further aspect, the compound exhibits inhibition of Nef-Hck activity with an IC₅₀ of less than about 10 μM. In yet a further aspect, the compound exhibits inhibition of Nef-Hck activity with an IC₅₀ of less than about 5 μM. In an even further aspect, the compound exhibits inhibition of Nef-Hck activity with an IC₅₀ of less than about 1 μM. In a still further aspect, the compound exhibits inhibition of Nef-Hck activity with an IC₅₀ of less than about 0.5 μM.

In a further aspect, the subject is a mammal. In a still further aspect, the subject is a human.

In a further aspect, the subject has been diagnosed with a need for treatment of the disorder prior to the administering step. In a still further aspect, the method further comprises the step of identifying a subject in need of treatment of the disorder.

3. Methods of Inhibiting a Viral Infection in at Least One Cell

In one aspect, disclosed are methods for inhibiting a viral infection in at least one cell, the method comprising the step of contacting the at least one cell with an effective amount of at least one disclosed compound, or a pharmaceutically acceptable salt thereof.

In a further aspect, the cell is mammalian. In a still further aspect, the cell is human. In yet a further aspect, the cell has been isolated from a mammal prior to the contacting step.

In a further aspect, contacting is via administration to a mammal.

4. Use of Compounds

In one aspect, the invention relates to the use of a disclosed compound or a product of a disclosed method. In a further aspect, a use relates to the manufacture of a medicament for the treatment of a viral infection in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a viral infection in a subject. Also disclosed is the use of a compound for antagonism of a viral infection. In one aspect, the use is characterized in that the subject is a human. In one aspect, the use is characterized in that the disorder is a viral infection.

In a further aspect, the use relates to the manufacture of a medicament for the treatment of a viral infection in a subject.

In a further aspect, the use relates to antagonism of a viral infection in a subject. In a further aspect, the use relates to modulating viral activity in a subject. In a still further aspect, the use relates to modulating viral activity in a cell. In yet a further aspect, the subject is a human.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a viral infection in a mammal. In a further aspect, the viral infection is selected from chikungunya, Venezuelan equine encephalitis, dengue, influenza, and zika.

5. Manufacture of a Medicament

In one aspect, the invention relates to a method for the manufacture of a medicament for treating a viral infection in a subject having the viral infection, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the inhibition of a viral infection. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

6. Kits

In one aspect, disclosed are kits comprising at least one disclosed compound and one or more of (a) at least one antiviral agent; (b) a instructions for administering the at least one compound in connection with treating a viral infection; (c) instructions for administering the at least one compound in connection with reducing the risk of viral infection; and (d) instructions for treating a viral infection.

In a further aspect, the viral infection is selected from human immunodeficiency virus (HIV), human papillomavirus (HPV), influenza, chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, chikungunya, Venezuelan equine encephalitis, dengue, influenza, and zika. In a still further aspect, the viral infection is selected from chikungunya, Venezuelan equine encephalitis, dengue, influenza, and zika.

In a still further aspect, the antiviral agent is selected from selected from acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In a further aspect, the antiviral agent is an HIV therapeutic agent. In a still further aspect, the HIV therapeutic agent is selected from: a) a HIV fusion/lysis inhibitor, or a pharmaceutically acceptable prodrug, salt, solvate, or polymorph thereof; b) a HIV integrase inhibitor, or a pharmaceutically acceptable prodrug, salt, solvate, or polymorph thereof; c) a HIV non-nucleoside reverse transcriptase inhibitor, or a pharmaceutically acceptable prodrug, salt, solvate, or polymorph thereof; d) a HIV nucleoside reverse transcriptase inhibitor, or a pharmaceutically acceptable prodrug, salt, solvate, or polymorph thereof; and e) a HIV protease inhibitor, or a pharmaceutically acceptable prodrug, salt, solvate, or polymorph thereof.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, and elvitegravir, or a pharmaceutically acceptable prodrug, salt, solvate, or polymorph thereof. In a still further aspect, the HIV integrase inhibitor is raltegravir, or a pharmaceutically acceptable prodrug, salt, solvate, or polymorph thereof. In yet a further aspect, the HIV integrase inhibitor is dolutegravir, or a pharmaceutically acceptable prodrug, salt, solvate, or polymorph thereof. In an even further aspect, the HIV integrase inhibitor is elvitegravir, or a pharmaceutically acceptable prodrug, salt, solvate, or polymorph thereof.

In a further aspect, the HIV non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the HIV non-nucleoside reverse transcriptase inhibitor is delavirdine, or a pharmaceutically acceptable prodrug, salt, solvate, or polymorph thereof. In yet a further aspect, the HIV non-nucleoside reverse transcriptase inhibitor is efavirenz, or a pharmaceutically acceptable prodrug, salt, solvate, or polymorph thereof. In an even further aspect, the HIV non-nucleoside reverse transcriptase inhibitor is etravirine, or a pharmaceutically acceptable prodrug, salt, solvate, or polymorph thereof. In a still further aspect, the HIV non-nucleoside reverse transcriptase inhibitor is lersivirine, or a pharmaceutically acceptable prodrug, salt, solvate, or polymorph thereof. In yet a further aspect, the HIV non-nucleoside reverse transcriptase inhibitor is nevirapine, or a pharmaceutically acceptable prodrug, salt, solvate, or polymorph thereof. In an even further aspect, the HIV non-nucleoside reverse transcriptase inhibitor is rilpivirine, or a pharmaceutically acceptable prodrug, salt, solvate, or polymorph thereof.

In a further aspect, the HIV nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the HIV nucleoside reverse transcriptase inhibitor is selected from abacavir, didansine, elvucitabine, emtricitabine, lamivudine, stavudine, tenofovir, and zidovudine, or a pharmaceutically acceptable prodrug, salt, solvate, or polymorph thereof. In yet a further aspect, the HIV nucleoside reverse transcriptase inhibitor is abacavir, or a pharmaceutically acceptable prodrug, salt, solvate, or polymorph thereof. In an even further aspect, the HIV nucleoside reverse transcriptase inhibitor is didansine, or a pharmaceutically acceptable prodrug, salt, solvate, or polymorph thereof. In a still further aspect, the HIV nucleoside reverse transcriptase inhibitor is elvucitabine, or a pharmaceutically acceptable prodrug, salt, solvate, or polymorph thereof. In yet a further aspect, the HIV nucleoside reverse transcriptase inhibitor is emtricitabine, or a pharmaceutically acceptable prodrug, salt, solvate, or polymorph thereof. In an even further aspect, the HIV nucleoside reverse transcriptase inhibitor is lamivudine, or a pharmaceutically acceptable prodrug, salt, solvate, or polymorph thereof. In a still further aspect, the HIV nucleoside reverse transcriptase inhibitor is stavudine, or a pharmaceutically acceptable prodrug, salt, solvate, or polymorph thereof. In yet a further aspect, the HIV nucleoside reverse transcriptase inhibitor is tenofovir, or a pharmaceutically acceptable prodrug, salt, solvate, or polymorph thereof. In an even further aspect, the HIV nucleoside reverse transcriptase inhibitor is zidovudine, or a pharmaceutically acceptable prodrug, salt, solvate, or polymorph thereof.

In a further aspect, the HIV protease inhibitor is selected from wherein the HIV protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the HIV protease inhibitor is atazanir, or a pharmaceutically acceptable prodrug, salt, solvate, or polymorph thereof. In yet a further aspect, the HIV protease inhibitor is darunavir, or a pharmaceutically acceptable prodrug, salt, solvate, or polymorph thereof. In an even further aspect, the HIV protease inhibitor is fosamprenavir, or a pharmaceutically acceptable prodrug, salt, solvate, or polymorph thereof. In a still further aspect, the HIV protease inhibitor is indinavir, or a pharmaceutically acceptable prodrug, salt, solvate, or polymorph thereof. In yet a further aspect, the HIV protease inhibitor is lopinavir, or a pharmaceutically acceptable prodrug, salt, solvate, or polymorph thereof. In an even further aspect, the HIV protease inhibitor is nelfinavir, or a pharmaceutically acceptable prodrug, salt, solvate, or polymorph thereof. In a still further aspect, the HIV protease inhibitor is ritonavir, or a pharmaceutically acceptable prodrug, salt, solvate, or polymorph thereof. In yet a further aspect, the HIV protease inhibitor is saquinavir, or a pharmaceutically acceptable prodrug, salt, solvate, or polymorph thereof. In an even further aspect, the HIV protease inhibitor is tipranavir, or a pharmaceutically acceptable prodrug, salt, solvate, or polymorph thereof.

In a further aspect, the at least one compound and the at least one agent are co-formulated. In a further aspect, the at least one compound and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. General Experimental

The reactions were performed under a dry argon atmosphere and reaction temperatures were measured externally. Anhydrous solvents over molecular sieves were purchased from Aldrich and used as such in reactions. Purification of compounds was performed on an Isco Teledyne Combiflash Rf200 with four channels to carryout sequential purification. Universal RediSep solid sample loading pre-packed cartridges (5.0 g Silica) were used to absorb crude product and purified on 12 g silica RediSep Rf Gold Silica (20-40 μm spherical silica) columns using appropriate solvent gradients. Pure samples were dried overnight under high vacuum over P₂O₅ at 78° C. before analyses. The reactions were monitored by thin-layer chromatography (TLC) on pre-coated silica gel (60F₂₅₄) aluminium plates (0.25 mm) from E. Merck and visualized using UV light (254 nm). Pure samples were dried overnight under high vacuum over P₂O₅ at 78° C. before analyses. The HR-mass spectral data were obtained on an Agilent LC-MSTOF by electrospray ionization (ESI). ¹H NMR spectra were recorded at 400 MHz on Agilent/Varian MR-400 spectrometer in CDCl₃ or DMSO-d₆ as solvents. The chemical shifts (6) are in ppm downfield from standard tetramethylsilane (TMS). Coupling constants (J) are reported in Hertz (Hz). Purity of final compounds was checked by HPLC using Waters HPLC equipped with a 3100 Mass Detector using Sunfire C18 column (5 μm, 4.6×150 mm) using Acetonitrile-H₂O (both containing 0.1% formic acid) 10-90% in 15 min.

2. Biological Assay Descriptions

a. Surface Plasmon Resonance (SPR)

Interaction of inhibitor analogs with recombinant purified Nef proteins was performed using an SPR-based binding assay on a Reichert 4-channel SPR instrument. Full-length recombinant Nef proteins (HIV-1 Nef-SF2, HIV-1 Nef NL4-3 and SIV Nef-mac239) were expressed in E. coli and purified via N-terminal His-6 tags, ion-exchange chromatography, and gel filtration. Purity and identity of the Nef proteins was confirmed by SDS-PAGE and mass spectrometry. For SPR, Nef proteins were immobilized on carboxymethyl dextran biosensor chips (Reichert) in HBS-EP running buffer (10 mM HEPES, pH 7.4, 150 mM NaCl, 3 mM EDTA, 0.05% v/v Surfactant P20). Test compounds were injected in triplicate over a range of concentrations (typically 0.1 to 30 μM) at a rate of 10 μL/min for one min until equilibrium was reached, followed by dissociation in HBS-EP for 2 min. Kinetic rate constants were calculated from reference-corrected sensorgrams using the TraceDrawer software package (Reichert), and were best fit via 1:1 Langmuir or 2-step induced-fit binding models. Some initial SPR experiments were performed on a BIAcore T100 instrument (GE Healthcare) using four-channel CM5 biosensor chips, and ligand-protein interactions were best-fit using the BIAevaluations software (version 2.0.4).

b. HIV-1 Infectivity Assay

The antiretroviral activity of Nef inhibitor analogs was assessed using the reporter cell line, TZM-bl, which are HeLa cells engineered to express CD4, HIV-1 co-receptors, as well as an HIV-1 LTR-luciferase reporter gene. TZM-bl cells (2.5×10⁴) were plated in 96-well plates and allowed to adhere overnight prior to infection. Adherent cells and HIV-1_{NL4-3} (typically 5,000 pg p24/ml) were then incubated separately with compounds for 3 h and combined in a final volume of 200 μl in each well. Following incubation for 48 h at 37° C., the cells were lysed in 50 μl luciferase cell culture lysis reagent (Promega). Lysates (40 μl) were then transferred to white 96-well plates followed by 50 μl injections of luciferase reagent per well (Promega). Luminescence was then recorded with a delay time of 2 s and an integration period of 10 s. Cytotoxicity of each compound in the absence of HIV-1 was evaluated in TZM-bl cells using the CellTiter-Blue cell viability assay (Promega).

3. Chemistry Experimental

### a. General Synthesis of Amino-(Substituted Phenyl)-Carbonyl Amine (C1-C9)

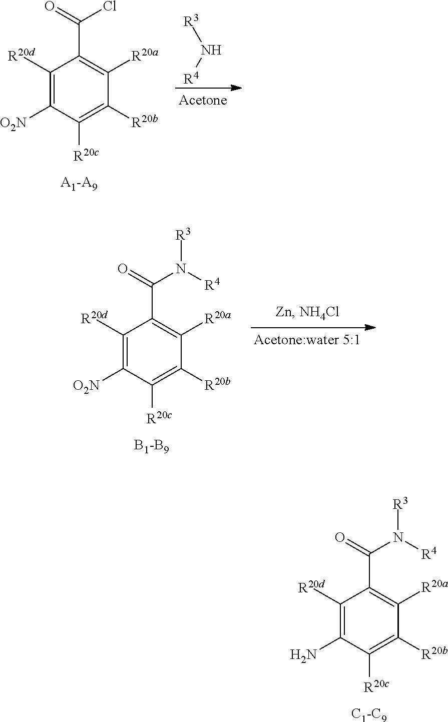

(1) Preparation of Nitro-Substituted Phenyl-Carbonyl Amine (B1-B9)

To a solution of substituted nitro benzoyl chloride A1-A9 (4.5 mmol) in acetone (5.0 mL) was added a solution of appropriate amine (0.992 mg, 2.5 eq.) in acetone (1.0 mL) at RT. The mixture was stirred in a sealed vial for 0.5 h. Upon complete consumption of starting material, the reaction mixture was concentrated in vacuo to afford a yellow oil. The oil was triturated in (1:1) Et₂O/hexanes or chromatographed to liberate a pale yellow solid B1-B9.

### (a) (2-chloro-5-nitrophenyl)(morpholino)methanone B1

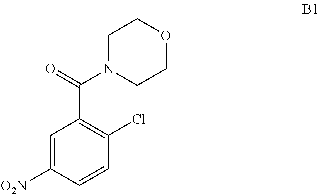 [SMILES: O=C(c1cc([N+](=O)[O-])ccc1Cl)N1CCOCC1]

Yield: 80%; 1H NMR (400 MHz, DMSO-d6) δ 8.32-8.21 (m, 2H), 7.83 (d, J=8.8 Hz, 1H), 3.76-3.41 (m, 4H), 3.63-3.41 (m, 2H), 3.23-3.04 (m, 2H).

### (b) 2-chloro-N-(4-chlorophenyl)-5-nitrobenzamide B2

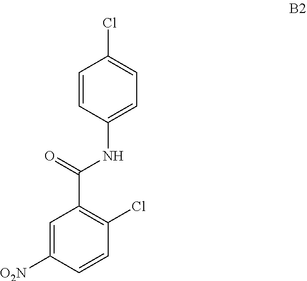 [SMILES: O=C(Nc1ccc(Cl)cc1)c1cc([N+](=O)[O-])ccc1Cl]

Yield: 91%; 1H NMR (400 MHz, DMSO-d6) δ 10.85 (s, 1H), 8.49 (d, J=2.7 Hz, 1H), 8.35 (dd, J=8.8, 2.8 Hz, 1H), 7.90 (d, J=8.8 Hz, 1H), 7.78-7.69 (m, 2H), 7.49-7.40 (m, 2H).

### (c) (2-chloro-5-nitrophenyl)(2,2-dimethylmorpholino)methanone B3

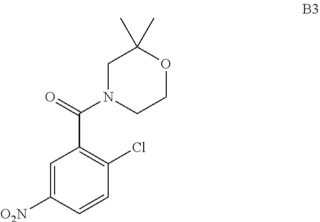 [SMILES: CC1(C)CN(C(=O)c2cc([N+](=O)[O-])ccc2Cl)CCO1]

Yield: 88%; 1H NMR (400 MHz, DMSO-d6) δ 8.28-8.22 (m, 2H), 7.86-7.80 (m, 1H), 3.76-3.46 (m, 4H), 3.16-2.98 (m, 2H), 1.22 (d, J=2.3 Hz, 3H), 1.05 (d, J=13.1 Hz, 3H).

### (d) (2-chloro-5-nitrophenyl)(pyrrolidin-1-yl)methanone B4

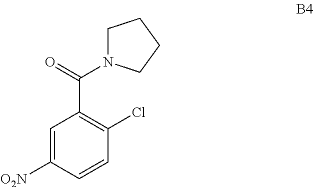 [SMILES: O=C(c1cc([N+](=O)[O-])ccc1Cl)N1CCCC1]

Yield: 86%; 1H NMR (400 MHz, Chloroform-d) δ 8.24-8.14 (m, 2H), 7.59 (dt, J=8.7, 0.4 Hz, 1H), 3.73-3.64 (m, 3H), 2.06-1.87 (m, 5H).

### (e) (2-chloro-5-nitrophenyl)(2,6-dimethylmorpholino)methanone B5

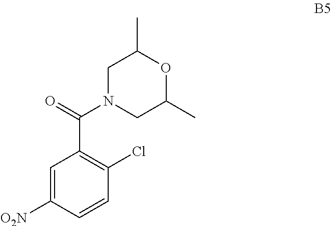 [SMILES: CC1CN(C(=O)c2cc([N+](=O)[O-])ccc2Cl)CC(C)O1]

Yield: 59%; 1H NMR (400 MHz, Chloroform-d) δ 8.23-8.13 (m, 2H), 7.64-7.58 (m, 1H), 4.60 (dddd, J=16.3, 13.3, 2.6, 1.7 Hz, 1H), 3.74-3.48 (m, 2H), 3.13-2.77 (m, 2H), 2.63-2.54 (m, 1H), 1.27 (dd, J=6.3, 1.5 Hz, 3H), 1.10 (dd, J=6.2, 2.6 Hz, 3H).

### (f) (2-chloro-5-nitrophenyl)(piperidin-1-yl)methanone B6

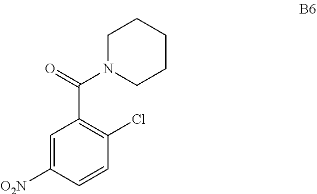 [SMILES: O=C(c1cc([N+](=O)[O-])ccc1Cl)N1CCCCC1]

Yield: 57%; 1H NMR (400 MHz, Chloroform-d) δ 8.21-8.12 (m, 2H), 7.62-7.54 (m, 1H), 3.85-3.68 (m, 3H), 3.27-3.10 (m, 3H), 1.70 (s, 3H), 1.74-1.66 (m, 1H).

### (g) (2-chloro-4-nitrophenyl)(morpholino)methanone B7

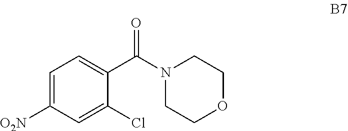 [SMILES: O=C(c1ccc([N+](=O)[O-])cc1Cl)N1CCOCC1]

Yield: 97%; 1H NMR (400 MHz, Chloroform-d) δ 8.30 (dd, J=2.2, 0.4 Hz, 1H), 8.19 (dd, J=8.4, 2.1 Hz, 1H), 7.50 (dd, J=8.4, 0.4 Hz, 1H), 3.93-3.84 (m, 1H), 3.82-3.78 (m, 3H), 3.77-3.58 (m, 2H), 3.29-3.16 (m, 2H).

### (h) (4-methyl-3-nitrophenyl)(morpholino)methanone B8

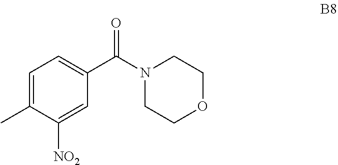 [SMILES: Cc1ccc(C(=O)N2CCOCC2)cc1[N+](=O)[O-]]

Yield: 88%; 1H NMR (400 MHz, Chloroform-d) δ 8.01 (dt, J=1.8, 0.4 Hz, 1H), 7.54 (ddd, J=7.8, 1.8, 0.4 Hz, 1H), 7.40 (dp, J=7.8, 0.6 Hz, 1H), 3.70 (s, 8H), 2.61 (dd, J=0.4 Hz, 3H).

### (i) (2-chloro-5-nitrophenyl)(4,4-difluoropiperidin-1-yl)methanone B9

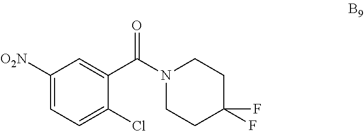 [SMILES: O=C(c1cc([N+](=O)[O-])ccc1Cl)N1CCC(F)(F)CC1]

Yield: 86%; 1H NMR (400 MHz, Chloroform-d) δ 8.23-8.17 (m, 2H), 7.62 (dd, J=8.7, 0.5 Hz, 1H), 4.13-3.97 (m, 1H), 3.85 (dt, J=13.3, 6.1 Hz, 1H), 3.36 (qdd, J=13.8, 7.4, 4.7 Hz, 2H), 2.17-1.86 (m, 4H).

(2) Preparation of Amino-(Substituted Phenyl)-Carbonyl Amine (C1-C9)

To a solution of nitro-substituted phenyl-carbonyl amine B1-B9 (970 mg, 3.58 mmol) in a 5:1 mixture of acetone:water (30 mL) was added ammonium chloride (2875 mg, 53.8 mmol) and zinc (2343 mg, 35.8 mmol) powder. The solution was stirred at RT for 10 minutes then filtered over celite and rinsed with acetone (5×5 mL). The solvent was removed in vacuo and the obtained crude was purified via flash chromatography to afford the desired aniline derivative C1-C9.

### (a) (5-amino-2-chlorophenyl)(morpholino)methanone C1

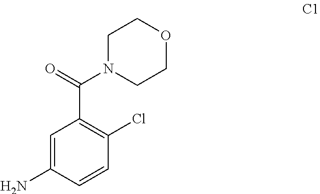 [SMILES: Nc1ccc(Cl)c(C(=O)N2CCOCC2)c1]

Yield: 77%; 1H NMR (400 MHz, DMSO-d6) δ 6.90 (dd, J=9.4, 8.8 Hz, 1H), 6.58 (ddd, J=8.8, 4.4, 2.9 Hz, 1H), 6.45 (dd, J=5.6, 2.9 Hz, 1H), 5.12 (s, 2H), 3.50 (t, J=4.8 Hz, 2H), 3.22 (t, J=4.7 Hz, 2H), 1.28-1.18 (m, 4H).

### (b) 5-amino-2-chloro-N-(4-chlorophenyl)benzamide C2

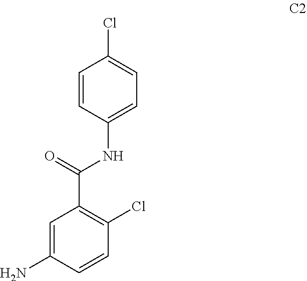 [SMILES: Nc1ccc(Cl)c(C(=O)Nc2ccc(Cl)cc2)c1]

Yield: 89%; 1H NMR (400 MHz, DMSO-d6) δ 10.50 (s, 1H), 7.78-7.68 (m, 2H), 7.44-7.35 (m, 2H), 7.13 (d, J=8.6 Hz, 1H), 6.72-6.61 (m, 2H), 5.48 (s, 2H).

### (c) (5-amino-2-chlorophenyl)(2,2-dimethylmorpholino)methanone C3

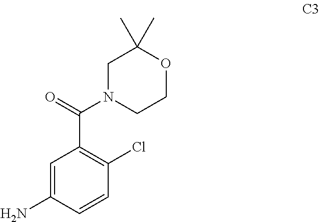 [SMILES: CC1(C)CN(C(=O)c2cc(N)ccc2Cl)CCO1]

Yield: 98%; 1H NMR (400 MHz, DMSO-d6) δ 7.07 (t, J=8.6 Hz, 1H), 6.56 (ddd, J=8.6, 4.9, 2.7 Hz, 1H), 6.45 (dd, J=6.2, 2.7 Hz, 1H), 5.40 (d, J=3.0 Hz, 2H), 3.87-3.46 (m, 4H), 3.14-2.94 (m, 2H), 1.23-1.03 (m, 6H); LCMS (M+H): 269.

### (d) (5-amino-2-chlorophenyl)(pyrrolidin-1-yl)methanone C4

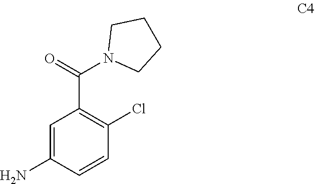 [SMILES: Nc1ccc(Cl)c(C(=O)N2CCCC2)c1]

Yield: 85%; 1H NMR (400 MHz, DMSO-d6) δ 7.05 (d, J=8.6 Hz, 1H), 6.55 (dd, J=8.7, 2.8 Hz, 1H), 6.43 (s, 1H), 5.36 (s, 2H), 3.40 (t, J=6.7 Hz, 2H), 3.09 (d, J=6.6 Hz, 2H), 1.88-1.75 (m, 4H); LCMS (M+H): 225.

### (e) (5-amino-2-chlorophenyl)(2,6-dimethylmorpholino)methanone C5

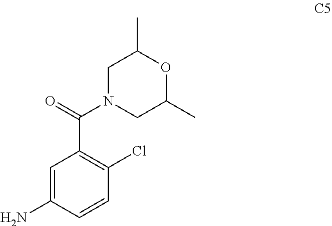 [SMILES: CC1CN(C(=O)c2cc(N)ccc2Cl)CC(C)O1]

Yield: 90%; 1H NMR (400 MHz, Chloroform-d) δ 7.15-7.09 (m, 1H), 6.64-6.49 (m, 2H), 3.80-3.43 (m, 3H), 3.23 (dddd, J=13.3, 9.1, 2.6, 1.8 Hz, 1H), 2.79 (ddd, J=71.7, 13.2, 10.6 Hz, 2H), 2.59-2.48 (m, 1H), 1.24 (dd, J=6.3, 2.2 Hz, 3H, 1.09 (d, J=6.2 Hz, 3H); LCMS (M+H): 269.

### (f) (5-amino-2-chlorophenyl)(piperidin-1-yl)methanone C6

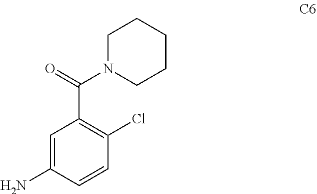 [SMILES: Nc1ccc(Cl)c(C(=O)N2CCCCC2)c1]

Yield: 61%; 1H NMR (400 MHz, Chloroform-d) δ 7.05 (dd, J=8.2, 0.3 Hz, 1H), 6.67-6.64 (m, 1H), 6.56 (dd, J=8.2, 2.2 Hz, 1H), 3.86-3.53 (m, 8H), 3.29 (d, J=27.5 Hz, 2H); LCMS (M+H): 241.

### (g) (4-amino-2-chlorophenyl)(morpholino)methanone C7

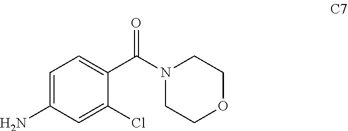 [SMILES: Nc1ccc(C(=O)N2CCOCC2)c(Cl)c1]

Yield: 93%; 1H NMR (400 MHz, Chloroform-d) δ 7.03 (ddt, J=7.5, 0.9, 0.5 Hz, 1H), 6.72-6.64 (m, 2H), 3.75 (d, J=67.7 Hz, 11H), 2.15 (dd, J=0.8, 0.4 Hz, 3H); LCMS (M+H): 221.

### (h) (3-amino-4-methylphenyl)(morpholino)methanone C8

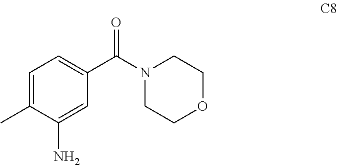 [SMILES: Cc1ccc(C(=O)N2CCOCC2)cc1N]

Yield: 92%; 1H NMR (400 MHz, Chloroform-d) δ 7.03 (ddt, J=7.5, 0.9, 0.5 Hz, 1H), 6.72-6.64 (m, 2H), 3.75 (d, J=67.7 Hz, 11H), 2.15 (dd, J=0.8, 0.4 Hz, 3H); LCMS (M+H): 221.

### (i) (5-amino-2-chlorophenyl)(4,4-difluoropiperidin-1-yl)methanone C9

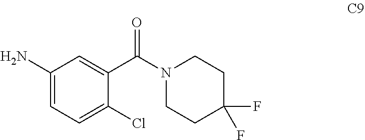 [SMILES: Nc1ccc(Cl)c(C(=O)N2CCC(F)(F)CC2)c1]

Yield: 98%; 1H NMR (400 MHz, Chloroform-d) δ 7.16-7.12 (m, 1H), 6.63 (dd, J=8.6, 2.8 Hz, 1H), 6.56 (dd, J=2.8, 0.3 Hz, 1H), 4.08-4.00 (m, 1H), 3.81-3.74 (m, 1H), 3.51-3.28 (m, 2H), 2.15-2.00 (m, 4H), 1.96-1.83 (m, 1H); LCMS (M+H): 275.

### b. Synthesis of 4-methyl-3-(pyrrolidin-1-ylsulfonyl)aniline F

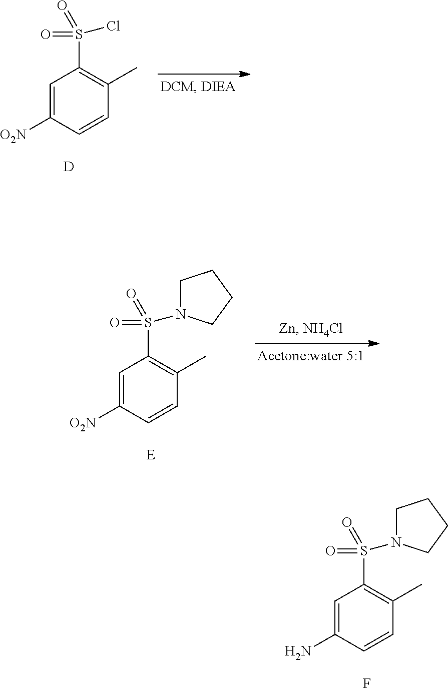

### (1) Preparation of 1-((2-methyl-5-nitrophenyl)sulfonyl)pyrrolidine E

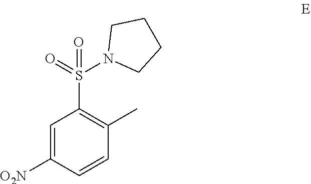 [SMILES: Cc1ccc([N+](=O)[O-])cc1S(=O)(=O)N1CCCC1]

To as solution of 5-nitro-4-methyl phenyl sulfonyl chloride D (4.24 mmol) in DCM (7 mL) at 0° C. was added pyrrolidine (4.24 mmol) followed by DIPEA (0.741 ml, 4.24 mmol). The reaction mixture was stirred at RT for 16 h. The obtained crude material, after solvent removal, was purified via biotage flash chromatography to give the desired nitro-(substituted phenyl)-sulfonyl amine E. Yield: 96%; 1H NMR (400 MHz, DMSO-d6) δ 8.46 (dt, J=2.5, 0.4 Hz, 1H), 8.43-8.35 (m, 1H), 7.81-7.72 (m, 1H), 3.31-3.19 (m, 3H), 3.17 (d, J=5.2 Hz, 1H), 2.69 (d, J=0.6 Hz, 3H), 1.94-1.79 (m, 4H); FABMS (M+H) calculated for C₁₁H₁₄N₂O₄S.H was 271.0747 found 271.0746.

### (2) Preparation of 4-methyl-3-(pyrrolidin-1-ylsulfonyl)aniline F

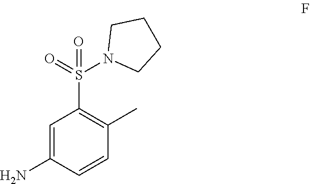 [SMILES: Cc1ccc(N)cc1S(=O)(=O)N1CCCC1]

Same procedure described for the preparation of C. Yield: 89%; 1H NMR (400 MHz, DMSO-d6) δ 7.07-6.99 (m, 2H), 6.69 (dd, J=8.1, 2.5 Hz, 1H), 5.36 (s, 2H), 3.20-3.09 (m, 4H), 2.35 (s, 3H), 1.88-1.74 (m, 4H); LCMS (M+H): 241.

### c. General Synthesis of (Substituted Phenyl)isothiazolo[5,4-b]pyridin-3(2H)-one (Compounds 1-8)

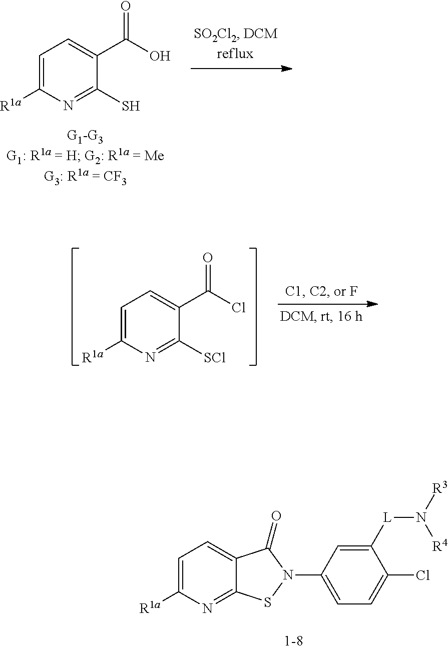

To a solution of 2-mercaptonicotinic acid derivative G1-G3 (6.44 mmol) in DCM (20 ml) thionyl chloride (1 mL) was added and the solution was refluxed for 2.5 h. After solvent removal, the yellow solid was dissolved in DCM (20 ml), then appropriate aniline C1-C9 or F (6.44 mmol) was added dropwise and the reaction mixture was stirred overnight at r.t. Water was added and the mixture was filtered, extracted with DCM and chromatographed to afford the desiret compound 1-8 as a white solid.

### (1) 2-(4-fluoro-3-(morpholine-4-carbonyl)phenyl)isothiazolo[5,4-b]pyridin-3(2H)-one 1

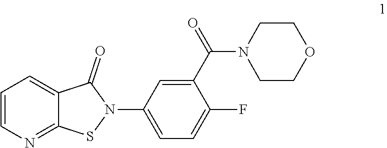 [SMILES: O=C(c1cc(-n2sc3ncccc3c2=O)ccc1F)N1CCOCC1]

Yield: 17%; 1H NMR (400 MHz, DMSO-d6) δ 7.86-7.74 (m, 1H), 7.59 (dd, J=7.9, 4.7 Hz, 1H), 3.64 (s, 2H), 3.54 (s, 1H), 3.27 (t, J=4.8 Hz, 1H), 2.90 (s, 1H), 2.85 (s, 1H), 2.66 (s, 4H), 1.22 (q, J=7.4, 6.6 Hz, 2H); LCMS (M+H): 360; HPLC purity >99 (% of AUC), t_{R}=6.3 minutes.

### (2) 2-(4-fluoro-3-(morpholine-4-carbonyl)phenyl)-6-methylisothiazolo[5,4-b]pyridin-3(2H)-one 2

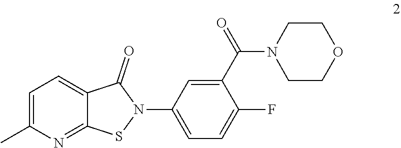 [SMILES: Cc1ccc2c(=O)n(-c3ccc(F)c(C(=O)N4CCOCC4)c3)sc2n1]

Yield: 5%; 1H NMR (400 MHz, DMSO-d6) δ 8.25 (d, J=8.1 Hz, 1H), 7.85-7.72 (m, 2H), 7.53-7.41 (m, 2H), 3.64 (bs, 4H), 3.54 (t, J=4.8 Hz, 2H), 3.27 (t, J=4.8 Hz, 2H), 2.65 (s, 3H); FABMS (M+H) calculated for C₁₈H₁₆FN₃O₃S.H was 374.0969 found 374.0958; HPLC purity >99 (% of AUC), t_{R}=8.84 minutes.

### (3) 2-(4-chloro-3-(morpholine-4-carbonyl)phenyl)-6-methylisothiazolo[5,4-b]pyridin-3(2H)-one 3

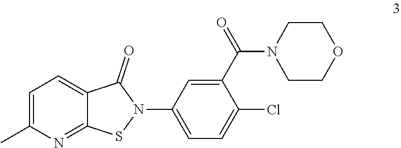 [SMILES: Cc1ccc2c(=O)n(-c3ccc(Cl)c(C(=O)N4CCOCC4)c3)sc2n1]

Yield: 18%; 1H NMR (400 MHz, DMSO-d6) δ 8.25 (d, J=8.1 Hz, 1H), 7.85-7.75 (m, 2H), 7.72-7.64 (m, 1H), 7.45 (d, J=8.1 Hz, 1H), 3.74-3.58 (m, 4H), 3.54 (dt, J=6.5, 4.5 Hz, 2H), 3.17 (q, J=4.1 Hz, 2H), 2.65 (s, 3H); FABMS (M+H) calculated for C₁₈H₁₆ClN₃O₃S.H was 390.0674 found 390.0677; HPLC purity >94 (% of AUC), t_{R}=9.47 minutes.

### (4) 6-methyl-2-(4-methyl-3-(pyrrolidin-1-ylsulfonyl)phenyl)isothiazolo[5,4-b]pyridin-3(2H)-one 4

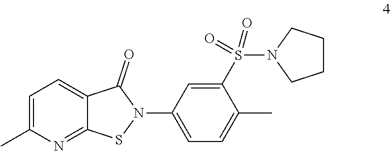 [SMILES: Cc1ccc2c(=O)n(-c3ccc(C)c(S(=O)(=O)N4CCCC4)c3)sc2n1]

Yield: 5%; 1H NMR (400 MHz, DMSO-d6) δ 8.25 (d, J=8.1 Hz, 1H), 8.20 (d, J=2.4 Hz, 1H), 7.79 (dd, J=8.2, 2.4 Hz, 1H), 7.59 (d, J=8.3 Hz, 1H), 7.46 (d, J=8.1 Hz, 1H), 3.30-3.21 (m, 4H), 2.65 (s, 3H), 2.58 (s, 3H), 1.92-1.7μ9 (m, 4H); FABMS (M+H) calculated for C₁₈H₁₆FN₃O₃S.H was 390.0941 found 394.0942; HPLC purity >99 (% of AUC), t_{R}=8.84 minutes.

### (5) 2-(4-chloro-3-(morpholine-4-carbonyl)phenyl)isothiazolo[5,4-b]pyridin-3(2H)-one 5

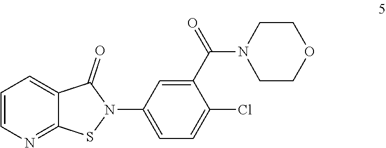 [SMILES: O=C(c1cc(-n2sc3ncccc3c2=O)ccc1Cl)N1CCOCC1]

Yield: 7%; 1H NMR (400 MHz, DMSO-d6) δ 8.94 (dd, J=4.7, 1.6 Hz, 1H), 8.41 (dd, J=7.9, 1.7 Hz, 1H), 7.82 (dd, J=7.0, 2.5 Hz, 2H), 7.75-7.68 (m, 1H), 7.61 (dd, J=7.9, 4.7 Hz, 1H), 3.74-3.61 (m, 4H), 3.57 (q, J=4.6 Hz, 2H), 3.19 (s, 2H); FABMS (M+H) calculated for C₁₇H₁₄CN₃O₃S.H was 376.0517 found 376.0508; HPLC purity >99 (% of AUC), t_{R}=7.46 minutes.

### (6) 2-(4-methyl-3-(pyrrolidin-1-ylsulfonyl)phenyl)isothiazolo[5,4-b]pyridin-3(2H)-one 6

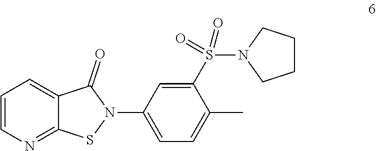 [SMILES: Cc1ccc(-n2sc3ncccc3c2=O)cc1S(=O)(=O)N1CCCC1]

Yield: 7%; 1H NMR (400 MHz, DMSO-d6) δ 8.94 (dd, J=4.7, 1.6 Hz, 1H), 8.41 (dd, J=7.9, 1.7 Hz, 1H), 7.82 (dd, J=7.0, 2.5 Hz, 2H), 7.75-7.68 (m, 1H), 7.61 (dd, J=7.9, 4.7 Hz, 1H), 3.74-3.61 (m, 4H), 3.57 (q, J=4.6 Hz, 2H), 3.19 (s, 2H); FABMS (M+H) calculated for C₁₇H₁₇N₃O₃S₂.H was 376.0784 found 376.0785; HPLC purity >96 (% of AUC), t_{R}=5.05 minutes.

### (7) 2-chloro-N-(4-chlorophenyl)-5-(3-oxoisothiazolo[5,4-b]pyridin-2(3H)-yl)benzamide 7

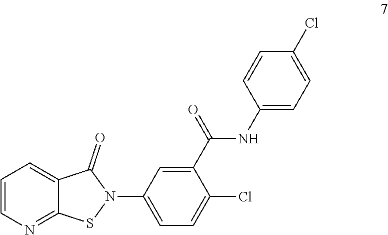 [SMILES: O=C(Nc1ccc(Cl)cc1)c1cc(-n2sc3ncccc3c2=O)ccc1Cl]

Yield: 7%; 1H NMR (400 MHz, DMSO-d6) δ 10.78 (s, 1H), 8.93 (dd, J=4.7, 1.7 Hz, 1H), 8.40 (dd, J=7.9, 1.7 Hz, 1H), 7.96 (dd, J=2.7, 0.4 Hz, 1H), 7.87 (dd, J=8.7, 2.7 Hz, 1H), 7.78-7.69 (m, 3H), 7.60 (dd, J=7.9, 4.7 Hz, 1H), 7.46-7.37 (m, 2H); FABMS (M+H) calculated for C₁₉H₁₁Cl₂N₃O₂S2.H was 416.0022 found 416.0018; HPLC purity >94 (% of AUC), t_{R}=11.8 minutes.

### (8) 2-(4-chloro-3-(morpholine-4-carbonyl)phenyl)-6-(trifluoromethyl)isothiazolo[5,4-b]pyridin-3(2H)-one

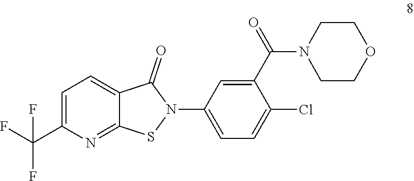 [SMILES: O=C(c1cc(-n2sc3nc(C(F)(F)F)ccc3c2=O)ccc1Cl)N1CCOCC1]

Yield: 45%; 1H NMR (400 MHz, DMSO-d6) δ 8.71-8.63 (m, 1H), 8.07 (d, J=8.1 Hz, 1H), 7.86-7.78 (m, 2H), 7.74 (dt, J=9.0, 1.3 Hz, 1H), 3.74-3.61 (m, 4H), 3.57 (dt, J=6.3, 3.6 Hz, 2H), 3.20 (dt, J=4.3, 2.4 Hz, 2H); FABMS (M+H) calculated for C₁₈H₁₃ClF₃N₃O₃S₂.H was 444.0391 found 444.0380; HPLC purity >99 (% of AUC), t_{R}=10.3 minutes.

### d. General Synthesis of (Substituted phenyl)isothiazolo[5,4-b]pyridin-3(2H)-one 9-16 (Compounds 9-16)

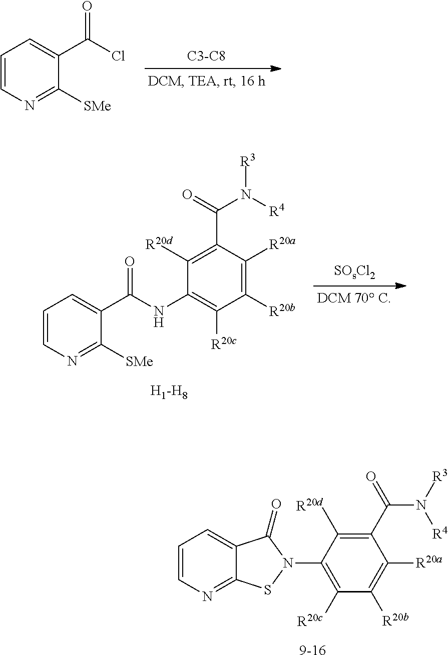

### (1) Preparation of 2-methylthio-nicotinamide Derivatives H1-H8

2-Methylthio-nicotinoyl chloride (300 mg, 1.599 mmol) was dissolved in DCM (6 ml) and appropriate aniline and TEA (0.223 ml, 1.599 mmol) were added at 0° C. The reaction mixture was stirred overnight at RT, concentrated and purified to afford 2-methylthio-nicotinamide intermediates H1-H8.

### (a) 2-(methylthio)-N-(4-(pyrrolidine-1-carbonyl)phenyl)nicotinamide H1

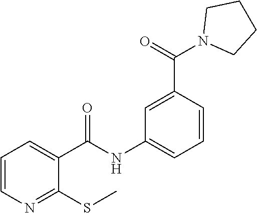 [SMILES: CSc1ncccc1C(=O)Nc1cccc(C(=O)N2CCCC2)c1]

Yield: 59%; 1H NMR (400 MHz, DMSO-d6) δ 10.60 (s, 1H), 8.58 (dd, J=4.9, 1.8 Hz, 1H), 7.91 (dd, J=7.6, 1.8 Hz, 1H), 7.76-7.71 (m, 2H), 7.55-7.50 (m, 2H), 7.25 (dd, J=7.6, 4.9 Hz, 1H), 3.43 (d, J=9.0 Hz, 4H), 2.46 (s, 3H), 1.82 (dq, J=13.0, 6.5 Hz, 4H); LCMS (M+H): 442.

### (b) 2-(methylthio)-N-(4-(morpholine-4-carbonyl)phenyl)nicotinamide H2

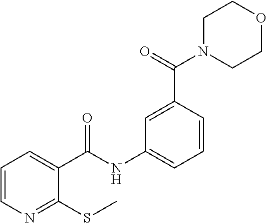 [SMILES: CSc1ncccc1C(=O)Nc1cccc(C(=O)N2CCOCC2)c1]

Yield: 49%; 1H NMR (400 MHz, DMSO-d6) δ 10.57 (s, 1H), 8.58 (dd, J=4.9, 1.7 Hz, 1H), 7.91 (dd, J=7.6, 1.8 Hz, 1H), 7.79 (t, J=1.8 Hz, 1H), 7.72 (ddd, J=8.2, 2.2, 1.1 Hz, 1H), 7.44-7.39 (m, 1H), 7.25 (dd, J=7.6, 4.9 Hz, 1H), 7.13 (ddd, J=7.5, 1.6, 1.1 Hz, 1H), 3.59 (s, 8H), 2.46 (s, 3H).

### (c) N-(4-chloro-3-(2,2-dimethylmorpholine-4-carbonyl)phenyl)-2-(methylthio)nicotinamide H3

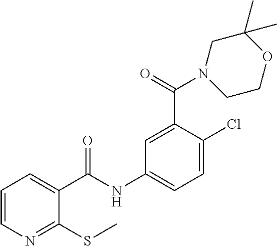 [SMILES: CSc1ncccc1C(=O)Nc1ccc(Cl)c(C(=O)N2CCOC(C)(C)C2)c1]

Yield: 75%; 1H NMR (400 MHz, DMSO-d6) δ 10.65 (s, 1H), 8.58 (dd, J=4.9, 1.8 Hz, 1H), 7.92 (ddd, J=7.6, 3.7, 1.8 Hz, 1H), 7.77-7.70 (m, 2H), 7.54-7.47 (m, 1H), 7.25 (dd, J=7.6, 4.9 Hz, 1H), 3.74-3.51 (m, 2H), 3.41-3.32 (m, 2H), 3.16-3.00 (m, 2H), 2.46 (s, 3H), 1.21 (d, J=11.8 Hz, 3H), 1.08-1.05 (m, 3H).

### (d) N-(4-chloro-3-(pyrrolidine-1-carbonyl)phenyl)-2-(methylthio)nicotinamide H4

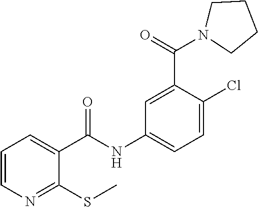 [SMILES: CSc1ncccc1C(=O)Nc1ccc(Cl)c(C(=O)N2CCCC2)c1]

Yield: 30%; 1H NMR (400 MHz, Chloroform-d) δ 8.89 (s, 1H), 8.55 (dd, J=4.8, 1.8 Hz, 1H), 7.97 (dd, J=7.7, 1.8 Hz, 1H), 7.73 (dd, J=8.7, 2.6 Hz, 1H), 7.52 (d, J=2.6 Hz, 1H), 7.34 (d, J=8.7 Hz, 1H), 7.10 (dd, J=7.7, 4.8 Hz, 1H), 3.56 (t, J=6.9 Hz, 2H), 3.25 (t, J=6.5 Hz, 2H), 2.61 (s, 3H), 2.00-1.86 (m, 2H); LCMS (M+H): 376.

### (e) N-(4-chloro-3-(2,6-dimethylmorpholine-4-carbonyl)phenyl)-2-(methylthio)nicotinamide H5

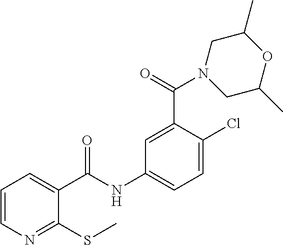 [SMILES: CSc1ncccc1C(=O)Nc1ccc(Cl)c(C(=O)N2CC(C)OC(C)C2)c1]

Yield: 52%; 1H NMR (400 MHz, Chloroform-d) δ 9.29 (d, J=3.7 Hz, 1H), 7.87-7.82 (m, 1H), 7.66 (td, J=8.8, 2.6 Hz, 1H), 7.48 (dd, J=27.5, 2.5 Hz, 1H), 7.31-7.25 (m, 1H), 7.09-6.98 (m, 2H), 4.55-4.44 (m, 2H), 3.74-3.55 (m, 4H), 2.89-2.66 (m, 2H), 1.25-1.22 (m, 3H), 1.07 (d, J=3.3 Hz, 3H); LCMS (M+H): 437.

### (f) N-(4-chloro-3-(piperidine-1-carbonyl)phenyl)-2-(methylthio)nicotinamide H6

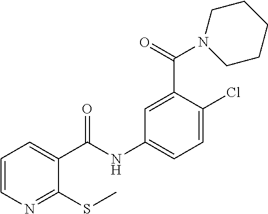 [SMILES: CSc1ncccc1C(=O)Nc1ccc(Cl)c(C(=O)N2CCCCC2)c1]

Yield: 98%; 1H NMR (400 MHz, Chloroform-d) δ 8.70 (s, 1H), 8.56 (dd, J=4.8, 1.8 Hz, 1H), 7.96 (dd, J=7.7, 1.8 Hz, 1H), 7.70 (dd, J=8.7, 2.6 Hz, 1H), 7.48 (d, J=2.6 Hz, 1H), 7.35 (d, J=8.7 Hz, 1H), 7.13-7.08 (m, 1H), 3.70 (d, J=5.5 Hz, 3H), 3.30-3.15 (m, 2H), 2.62 (s, 3H), 1.45-1.52 (m, 5H); LCMS (M+H): 390.

### (g) N-(2-methyl-5-(piperidine-1-carbonyl)phenyl)-2-(methylthio)nicotinamide H7

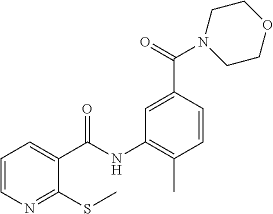 [SMILES: CSc1ncccc1C(=O)Nc1cc(C(=O)N2CCOCC2)ccc1C]

Yield: 52%; 1H NMR (400 MHz, Chloroform-d) δ 8.56 (dd, J=4.8, 1.8 Hz, 1H), 8.37 (s, 1H), 7.98-7.89 (m, 2H), 7.26-7.23 (m, 1H), 7.16-7.09 (m, 2H), 3.69 (s, 6H), 3.54 (s, 2H), 2.62 (s, 3H), 2.35 (d, J=0.6 Hz, 3H); LCMS (M+H): 372.

### (h) N-(4-chloro-3-(4,4-difluoropiperidine-1-carbonyl)phenyl)-2-(methylthio)nicotinamide H8

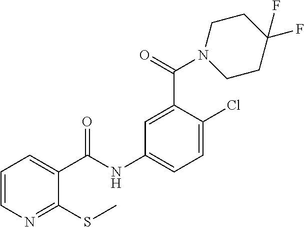 [SMILES: CSc1ncccc1C(=O)Nc1ccc(Cl)c(C(=O)N2CCC(F)(F)CC2)c1]

Yield: 48%; 1H NMR (400 MHz, Chloroform-d) δ 8.68 (s, 1H), 8.56 (dd, J=4.8, 1.8 Hz, 1H), 7.93 (dd, J=7.7, 1.8 Hz, 1H), 7.65-7.59 (m, 2H), 7.36 (d, J=8.6 Hz, 1H), 7.10 (dd, J=7.7, 4.8 Hz, 1H), 3.98 (dt, J=12.4, 5.8 Hz, 1H), 3.77 (dt, J=13.2, 6.2 Hz, 1H), 3.50-3.41 (m, 1H), 3.35 (ddd, J=13.6, 7.9, 4.4 Hz, 1H), 2.62 (s, 3H), 2.14-1.86 (m, 4H); LCMS (M+H): 426.

### (2) Preparation of Compounds 9-16

2-Methylthio-nicotinamide derivative (200 mg, 0.586 mmol) was suspended in DCM (5 ml) and sulfuryl chloride (0.048 ml, 0.586 mmol) was added. The reaction mixture was stirred at RT for 1 h then at 70° C. overnight. Concentrated and purified to afford the final target 9-16.

### (a) 2-(4-(pyrrolidine-1-carbonyl)phenyl)isothiazolo[5,4-b]pyridin-3(2H)-one 9

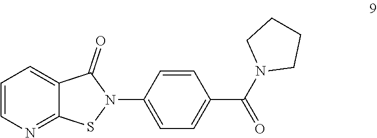 [SMILES: O=C(c1ccc(-n2sc3ncccc3c2=O)cc1)N1CCCC1]

Yield: 12%; 1H NMR (400 MHz, Chloroform-d) δ 8.81 (dd, J=4.7, 1.7 Hz, 1H), 8.34 (dd, J=7.9, 1.7 Hz, 1H), 7.81-7.76 (m, 2H), 7.67-7.62 (m, 2H), 7.41 (dd, J=7.9, 4.7 Hz, 1H), 3.66 (t, J=6.9 Hz, 2H), 3.47 (t, J=6.6 Hz, 2H), 2.01-1.86 (m, 4H); FABMS (M+H) calculated for C₁₇H₁₅N₃O₂S.H was 326.0958 found 326.0946; HPLC purity >90 (% of AUC), t_{R}=8.25 minutes.

### (b) 2-(3-(morpholine-4-carbonyl)phenyl)isothiazolo[5,4-b]pyridin-3(2H)-one 10

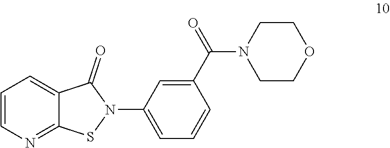 [SMILES: O=C(c1cccc(-n2sc3ncccc3c2=O)c1)N1CCOCC1]

Yield: 26%; 1H NMR (400 MHz, Chloroform-d) δ 8.81 (dd, J=4.7, 1.7 Hz, 1H), 8.34 (dd, J=7.9, 1.7 Hz, 1H), 7.81-7.76 (m, 2H), 7.67-7.62 (m, 2H), 7.41 (dd, J=7.9, 4.7 Hz, 1H), 3.66 (t, J=6.9 Hz, 2H), 3.47 (t, J=6.6 Hz, 2H), 2.01-1.86 (m, 4H); FABMS (M+H) calculated for C₁₇H₁₅N₃O₃S.H was 342.0907 found 342.0900; HPLC purity >97 (% of AUC), t_{R}=7.34 minutes.

### (c) 2-(4-chloro-3-(2,2-dimethylmorpholine-4-carbonyl)phenyl)isothiazolo[5,4-b]pyridin-3(2H)-one 11

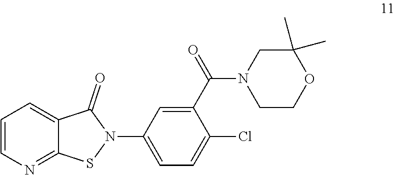 [SMILES: CC1(C)CN(C(=O)c2cc(-n3sc4ncccc4c3=O)ccc2Cl)CCO1]

Yield: 15%; 1H NMR (400 MHz, Chloroform-d) δ 8.83 (dd, J=4.7, 1.7 Hz, 1H), 8.33 (dt, J=7.9, 1.8 Hz, 1H), 7.75-7.67 (m, 2H), 7.52 (ddd, J=8.6, 2.0, 0.4 Hz, 1H), 7.43 (dd, J=7.9, 4.8 Hz, 1H), 4.12-3.75 (m, 3H), 3.71-3.06 (m, 4H), 1.33 (d, J=19.7 Hz, 4H), 1.20 (d, J=15.6 Hz, 3H); FABMS (M+H) calculated for C₁₉H₁₈ClN₃O₃S.H was 404.0830 found 404.0823; HPLC purity >93 (% of AUC), t_{R}=9.85 minutes.

### (d) 2-(4-chloro-3-(pyrrolidine-1-carbonyl)phenyl)isothiazolo[5,4-b]pyridin-3(2H)-one 12

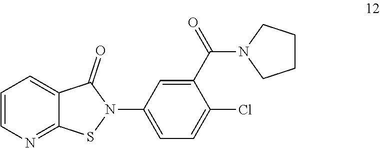 [SMILES: O=C(c1cc(-n2sc3ncccc3c2=O)ccc1Cl)N1CCCC1]

Yield: 6%; 1H NMR (400 MHz, Chloroform-d) δ 8.83 (dd, J=4.7, 1.7 Hz, 1H), 8.33 (dt, J=7.9, 1.8 Hz, 1H), 7.75-7.67 (m, 2H), 7.52 (ddd, J=8.6, 2.0, 0.4 Hz, 1H), 7.43 (dd, J=7.9, 4.8 Hz, 1H), 4.12-3.75 (m, 3H), 3.71-3.06 (m, 4H), 1.33 (d, J=19.7 Hz, 4H), 1.20 (d, J=15.6 Hz, 3H); LCMS (M+H): 360; HPLC purity >94 (% of AUC), t_{R}=9.39 minutes.

### (e) 2-(4-chloro-3-(2,6-dimethylmorpholine-4-carbonyl)phenyl)isothiazolo[5,4-b]pyridin-3(2H)-one 13

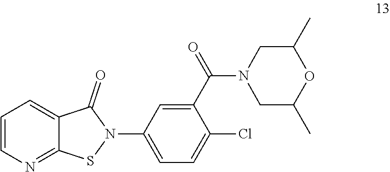 [SMILES: CC1CN(C(=O)c2cc(-n3sc4ncccc4c3=O)ccc2Cl)CC(C)O1]

Yield: 15%; 1H NMR (400 MHz, Chloroform-d) δ 8.81 (dd, J=4.7, 1.7 Hz, 1H), 8.31 (ddd, J=8.0, 1.8, 0.8 Hz, 1H), 7.76-7.62 (m, 2H), 7.50 (dd, J=8.6, 5.9 Hz, 1H), 7.41 (dd, J=7.9, 4.7 Hz, 1H), 4.65-4.54 (m, 1H), 3.77-3.49 (m, 2H), 3.23 (ddt, J=13.1, 4.3, 2.2 Hz, 1H), 2.86 (ddd, J=44.9, 13.2, 10.6 Hz, 1H), 2.56 (ddd, J=14.8, 13.3, 10.7 Hz, 1H), 1.25 (d, J=6.3 Hz, 3H), 1.10 (d, J=6.2 Hz, 3H); LCMS (M+H): 404; HPLC purity >96 (% of AUC), t_{R}=11.42 minutes.

### (f) 2-(4-chloro-3-(piperidine-1-carbonyl)phenyl)isothiazolo[5,4-b]pyridin-3(2H)-one 14

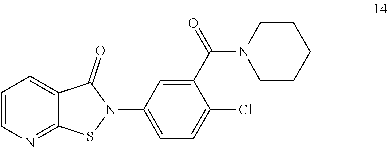 [SMILES: O=C(c1cc(-n2sc3ncccc3c2=O)ccc1Cl)N1CCCCC1]

Yield: 4%; 1H NMR (400 MHz, Chloroform-d) δ 8.82 (dd, J=4.7, 1.7 Hz, 1H), 8.33 (dd, J=7.9, 1.7 Hz, 1H), 7.72 (dd, J=8.7, 2.7 Hz, 1H), 7.65 (d, J=2.6 Hz, 1H), 7.50 (d, J=8.7 Hz, 1H), 7.42 (dd, J=7.9, 4.7 Hz, 1H), 3.82-3.70 (m, 2H), 3.33-3.18 (m, 2H), 1.71-1.66 (m, 4H), 1.51 (s, 2H); FABMS (M+H) calculated for C₁₈H₁₆C1N₃O₂S.H was 374.0725 found 374.0727; HPLC purity >97 (% of AUC), t_{R}=11.0 minutes.

### (g) 2-(2-methyl-5-(morpholine-4-carbonyl)phenyl)isothiazolo[5,4-b]pyridin-3(2H)-one 15

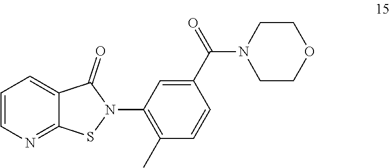 [SMILES: Cc1ccc(C(=O)N2CCOCC2)cc1-n1sc2ncccc2c1=O]

Yield: 9%; 1H NMR (400 MHz, Chloroform-d) δ 8.82 (dd, J=4.7, 1.7 Hz, 1H), 8.34 (dd, J=7.9, 1.7 Hz, 1H), 7.45-7.40 (m, 4H), 3.80-3.57 (m, 8H), 2.29 (d, J=0.5 Hz, 3H); FABMS (M+H) calculated for C₁₈H₁₇N₃O₃S.H was 356.1272 found 356.1074; HPLC purity >94 (% of AUC), t_{R}=11.54 minutes.

### (h) 6-(4-chloro-3-(4,4-difluoropiperidine-1-carbonyl)phenyl)-6,7-dihydro-5H-pyrrolo[3,4-b]pyridin-5-one 16

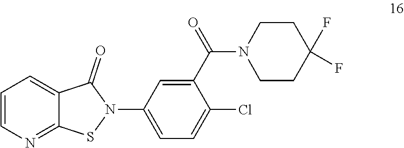 [SMILES: O=C(c1cc(-n2sc3ncccc3c2=O)ccc1Cl)N1CCC(F)(F)CC1]

Yield: 13%; 1H NMR (400 MHz, Chloroform-d) δ 8.81 (dd, J=4.7, 1.7 Hz, 1H), 8.31 (dd, J=7.9, 1.7 Hz, 1H), 7.75-7.69 (m, 2H), 7.55-7.49 (m, 1H), 7.42 (dd, J=7.9, 4.7 Hz, 1H), 4.02 (dt, J=12.3, 5.8 Hz, 1H), 3.84 (dt, J=13.1, 6.2 Hz, 1H), 3.51-3.45 (m, 1H), 3.43-3.35 (m, 1H), 2.14-1.91 (m, 4H); FABMS (M+H) calculated for C₁₈H₁₄ClF₂N₃O₂S.H was 410.0536 found 410.0541; HPLC purity >94 (% of AUC), t_{R}=11.42 minutes.

### e. Synthesis of 6-(4-chloro-3-(morpholine-4-carbonyl)phenyl)-6,7-dihydro-5H-pyrrolo[3,4-b]pyridin-5-one 17

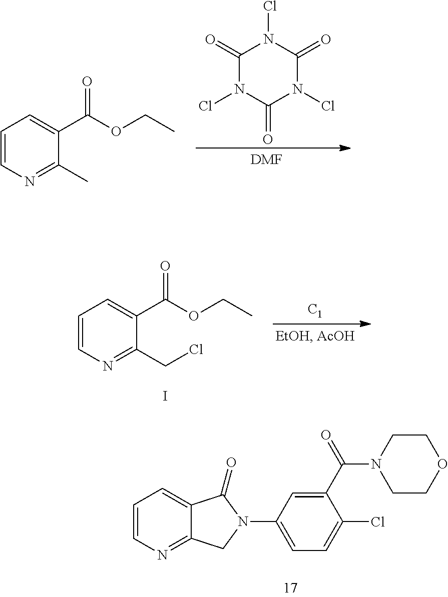

DMF (0.047 ml, 0.605 mmol) and trichloroisocyanuric acid (2.95 g, 12.71 mmol) were added to a solution of ester ethyl 2-methylnicotinate (1.000 g, 6.05 mmol) in DCM (7 ml) and resulted mixture was stirred 16 h at RT. Concentrated and purified on ISCO using 10-30% ethylactate in hexanes to give ethyl 2-(chloromethyl)nicotinate I in 99% yield (LCMS (M+H): 200). Ethyl 2-(chloromethyl)nicotinate I (100 mg, 0.501 mmol) and (5-amino-2-chlorophenyl)(morpholino) methanone (121 mg, 0.501 mmol) were dissolved in EtOH (5 ml) and AcOH (0.5 ml) and reaction mixture was refluxed overnight. Concentrated and purified on ISCO using 0-10% MeOH in DCM gave the desired product 17. Yield: 28%; 1H NMR (400 MHz, DMSO-d6) δ 8.83 (dd, J=4.9, 1.6 Hz, 1H), 8.19 (dd, J=7.7, 1.6 Hz, 1H), 8.02 (dd, J=8.9, 2.8 Hz, 1H), 7.97 (d, J=2.6 Hz, 1H), 7.62-7.55 (m, 2H), 5.16-5.01 (m, 2H), 3.66 (d, J=2.9 Hz, 4H), 3.55 (t, J=5.0 Hz, 2H), 3.17 (dd, J=6.4, 3.6 Hz, 2H); FABMS (M+H) calculated for C₁₈H₁₆ClN₃O₃.H was 358.0953 found 358.0953; HPLC purity >93 (% of AUC), t_{R}=9.85 minutes.

### 4. Evaluation of Antiretroviral Activity

A list of compounds evaluated for antiretroviral activity is shown in Table 1 below.

TABLE 1

| | | % Inhibition of |
|---|---|---|
| | | HIV-1 Infectivity at |
| | | 3 μM relative to |
| No. | Structure | ΔNef^{a} |
| 1 | 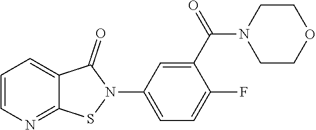 [SMILES: O=C(c1cc(-n2sc3ncccc3c2=O)ccc1F)N1CCOCC1] | 84 |
| 2 | 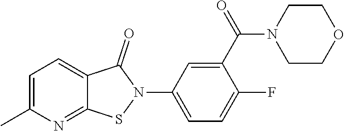 [SMILES: Cc1ccc2c(=O)n(-c3ccc(F)c(C(=O)N4CCOCC4)c3)sc2n1] | 52 |
| 3 | 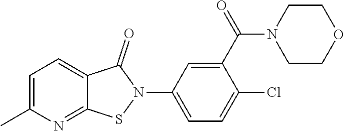 [SMILES: Cc1ccc2c(=O)n(-c3ccc(Cl)c(C(=O)N4CCOCC4)c3)sc2n1] | 23 |
| 4 | 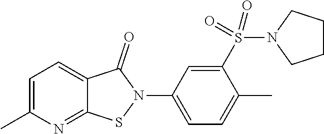 [SMILES: Cc1ccc2c(=O)n(-c3ccc(C)c(S(=O)(=O)N4CCCC4)c3)sc2n1] | 32 |
| 5 | 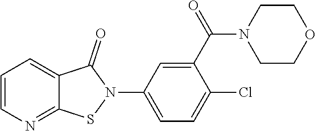 [SMILES: O=C(c1cc(-n2sc3ncccc3c2=O)ccc1Cl)N1CCOCC1] | 99 |
| 6 | 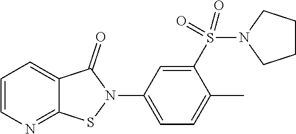 [SMILES: Cc1ccc(-n2sc3ncccc3c2=O)cc1S(=O)(=O)N1CCCC1] | 28 |
| 7 | 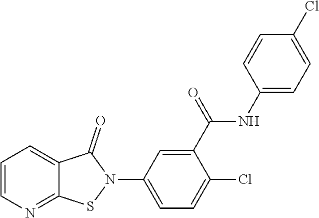 [SMILES: O=C(Nc1ccc(Cl)cc1)c1cc(-n2sc3ncccc3c2=O)ccc1Cl] | 58 |
| 8 | 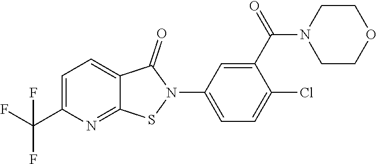 [SMILES: O=C(c1cc(-n2sc3nc(C(F)(F)F)ccc3c2=O)ccc1Cl)N1CCOCC1] | 71 |
| 9 | 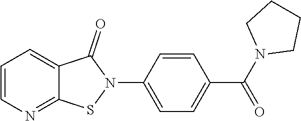 [SMILES: O=C(c1ccc(-n2sc3ncccc3c2=O)cc1)N1CCCC1] | 38 |
| 10 | 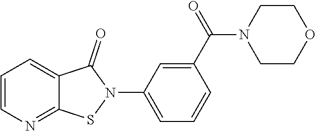 [SMILES: O=C(c1cccc(-n2sc3ncccc3c2=O)c1)N1CCOCC1] | 88 |
| 11 | 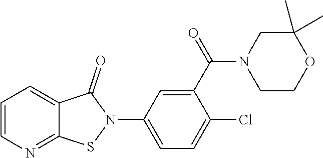 [SMILES: CC1(C)CN(C(=O)c2cc(-n3sc4ncccc4c3=O)ccc2Cl)CCO1] | 89 |
| 12 | 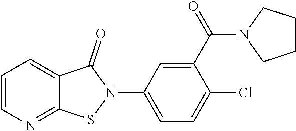 [SMILES: O=C(c1cc(-n2sc3ncccc3c2=O)ccc1Cl)N1CCCC1] | 100 |
| 13 | 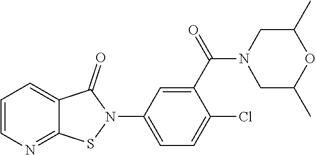 [SMILES: CC1CN(C(=O)c2cc(-n3sc4ncccc4c3=O)ccc2Cl)CC(C)O1] | 100 |
| 14 | 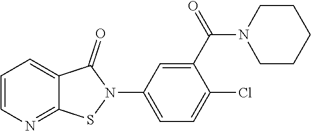 [SMILES: O=C(c1cc(-n2sc3ncccc3c2=O)ccc1Cl)N1CCCCC1] | 82 |
| 15 | 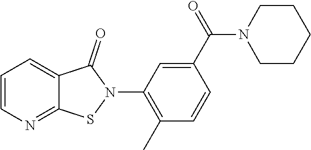 [SMILES: Cc1ccc(C(=O)N2CCCCC2)cc1-n1sc2ncccc2c1=O] | 81 |
| 16 | 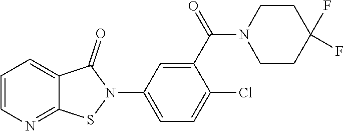 [SMILES: O=C(c1cc(-n2sc3ncccc3c2=O)ccc1Cl)N1CCC(F)(F)CC1] | 60 |
| 17 | 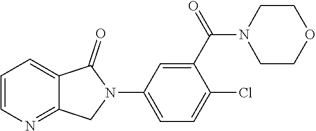 [SMILES: O=C(c1cc(N2Cc3ncccc3C2=O)ccc1Cl)N1CCOCC1] | 16 |

^{a}Infectivity was determined in TZM-bl reporter cells infected with HIV-1 NL4-3. No cytotoxicity was observed with any of these compounds at this concentration as determined by CellTiter-Blue cell viability assay (Promega).

### 5. Evaluation of Additional Properties

Table 2 below summarizes the ability of exemplary compounds to bind directly to recombinant purified HIV-1 and SIV Nef proteins in vitro, using the real-time technique of surface plasmon resonance (SPR).

TABLE 2

| | HIV-1 | HIV-1 | SIV Nef- | HIV-1 | HIV-1 | SIV Nef- |
|---|---|---|---|---|---|---|
| | Nef-SF2, | Nef-NL43, | mac239, | Nef-SF2, | Nef-NL43, | mac239, |
| No. | K_{D} (μM)¹ | K_{D}(μM)¹ | K_{D} (μM)¹ | K_{D} (μM)² | K_{D} (μM)² | K_{D} (μM)² |
| 1 | 1.03 | — | — | — | — | — |
| 2 | 0.50 | — | — | — | — | — |
| 3 | 0.87 | — | — | — | — | — |
| 4 | 0.26 | — | — | — | — | — |
| 5 | 0.162 | — | — | 2.24 | 7.33 | 1.51 |
| 6 | — | — | — | 2.40 | 8.66 | 1.60 |
| 7 | 0.12 | 0.19 | 0.28 | N/I | N/I | N/I |
| 8 | 0.11 | 0.31 | 0.78 | 2.34 | N/I | N/I |
| 9 | — | — | — | — | 0.04 | — |
| 10 | — | — | — | — | 0.25 | — |
| 11 | — | — | — | 0.22 | 0.21 | 1.69 |
| 12 | — | — | — | 1.71 | 0.96 | 3.66 |
| 13 | — | — | — | 0.38 | 0.59 | 1.41 |
| 14 | — | — | — | 1.40 | 1.76 | 3.11 |
| 15 | — | — | — | 0.39 | 0.14 | 1.28 |
| 16 | — | — | — | 9.39 | 56.2 | 21.1 |
| 17 | — | — | — | N/I | N/I | N/I |

¹Determined by SPR using Biacore T100 instrument.
²Determined by SPR using a Reichert 4-Channel instrument.
N/I, No Interaction up to 1 μM; a dash (—) indicates not tested.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 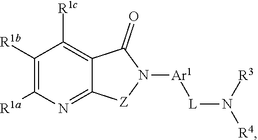
wherein Z is selected from O and S;
wherein L is selected from C═O and SO₂;
wherein each of R^{1a}, R^{1b}, and R^{1c} is independently selected from hydrogen, —NH₂, C1-C4 alkyl, C1-C4 haloalkyl, —CO₂H, —CO₂(C1-C4 alkyl), C1-C4 alkylamino, (C1-C4)(C1-C4) dialkylamino, and Cy¹;
wherein Cy¹, when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino;
wherein Ar¹ is selected from 6-membered monocyclic aryl and pyridinyl, and is substituted with 0, 1, 2, 3, or 4 R² groups;
wherein each occurrence of R², when present, is independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl);
or wherein any two adjacent R² groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl);
wherein R³ is hydrogen or C1-C4 alkyl;
wherein R⁴ is selected from C1-C8 alkyl, Cy², and (C1-C4)Cy²;
wherein Cy², when present, is selected from cycloalkyl, heterocycloalkyl, aryl, and heteroaryl, and is substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino;
or wherein each of R³ and R⁴ are covalently bonded together and, together with the intermediate atoms, comprise a 3- to 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino,
or a pharmaceutically acceptable salt thereof.

2. The compound of claim 1, wherein Z is S.

3. The compound of claim 1, wherein Ar¹ is selected from 6-membered monocyclic aryl and pyridinyl, and is substituted with 0 or 1 R² group.

4. The compound of claim 1, wherein each of R³ and R⁴ are covalently bonded together and, together with the intermediate atoms, comprise a 3- to 6-membered heterocycloalkyl substituted with 0, 1, 2, or 3 groups independently selected from halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino.

5. The compound of claim 1, wherein the compound has a structure represented by a formula: 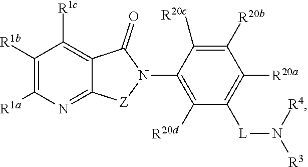
wherein each of R^{20a}, R^{20b}, R^{20c}, and R^{20d} is independently selected from hydrogen, halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl);
or wherein any two adjacent R^{20a}, R^{20b}, R^{20c}, and R^{20d} groups are covalently bonded together and, together with the intermediate atoms, comprise a 5- to 6-membered aryl or a 5- to 6-membered heteroaryl, and are substituted with 0, 1, 2, or 3 groups independently selected from halogen, —CN, —NH₂, —OH, C1-C4 alkyl, C1-C4 haloalkyl, C1-C4 hydroxyalkyl, C1-C4 alkoxy, —CO₂H, —CO₂(C1-C4 alkyl), —SO₂H, —SO₂(C1-C4 alkyl), C1-C4 alkylamino, (C1-C4)(C1-C4) dialkylamino, —CO₂NH₂, —CO₂NH(C1-C4 alkyl), —CO₂N(C1-C4 alkyl)(C1-C4 alkyl), —SO₂NH₂, —SO₂NH(C1-C4 alkyl), and —SO₂N(C1-C4 alkyl)(C1-C4 alkyl).

6. The compound of claim 5, wherein Z is S.

7. The compound of claim 5, wherein each of R^{20b}, R^{20c}, and R^{20d} is hydrogen.

8. The compound of claim 5, wherein the compound has a structure represented by a formula:

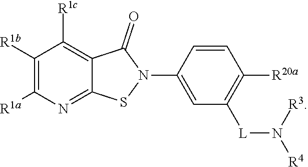

9. The compound of claim 5, wherein the compound has a structure represented by a formula:

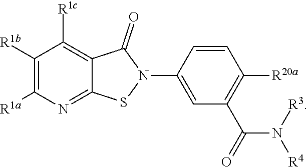

10. The compound of claim 1, wherein the compound has a structure represented by a formula: 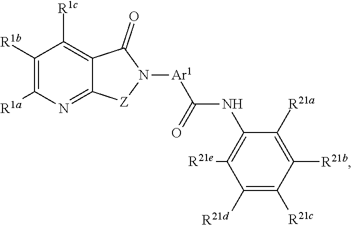
wherein each of R^{21a}, R^{21b}, R^{21c}, and R^{21d} is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino.

11. The compound of claim 10, wherein the compound has a structure represented by a formula:

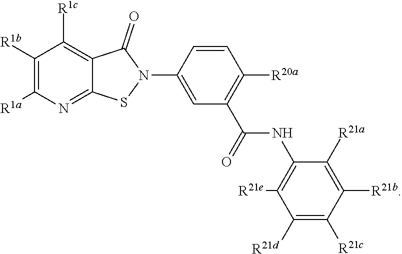

12. The compound of claim 1, wherein the compound has a structure represented by a formula: 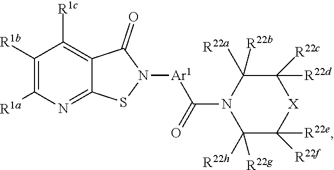
wherein X is selected from N, O, and CR^{30a}R^{30b};
wherein each of R^{30a} and R^{30b}, when present, is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino;
wherein each of R^{22a}, R^{22b}, R^{22c}, R^{22d}, R^{22e}, R^{22f}, R^{22g}, and R^{22h} is independently selected from hydrogen, halogen, —CN, —NH₂, —OH, C1-C4 alkyl, C1-C4 haloalkyl, C1-C4 cyanoalkyl, C1-C4 hydroxyalkyl, C1-C4 alkoxy, C1-C4 alkylamino, and (C1-C4)(C1-C4) dialkylamino.

13. The compound of claim 12, wherein the compound has a structure represented by a formula:

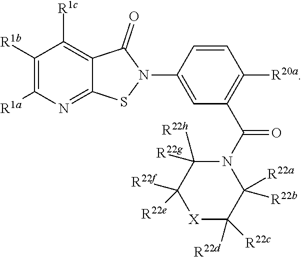

14. The compound of claim 12, wherein the compound has a structure represented by a formula:

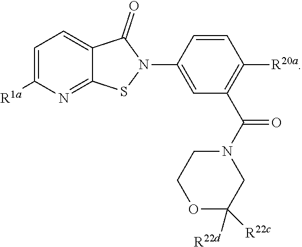

15. The compound of claim 1, wherein the compound is selected from:

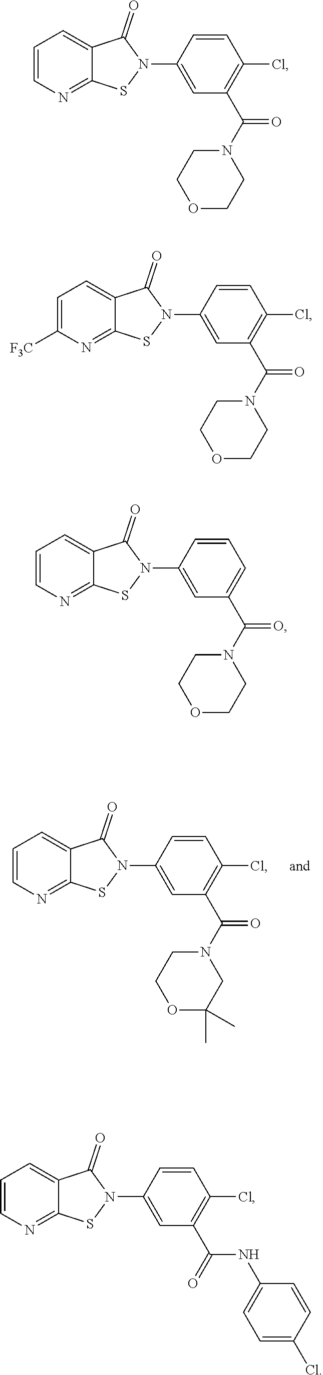 [SMILES: O=C(c1cc(-n2sc3ncccc3c2=O)ccc1Cl)N1CCOCC1 ; O=C(c1cc(-n2sc3nc(C(F)(F)F)ccc3c2=O)ccc1Cl)N1CCOCC1 ; O=C(c1cccc(-n2sc3ncccc3c2=O)c1)N1CCOCC1 ; CC1(C)CN(C(=O)c2cc(-n3sc4ncccc4c3=O)ccc2Cl)CCO1 ; "and" ; O=C(Nc1ccc(Cl)cc1)c1cc(-n2sc3ncccc3c2=O)ccc1Cl]

16. A pharmaceutical composition comprising a therapeutically effective amount of at least one compound of claim 1 and a pharmaceutically acceptable carrier.

17. A method for the treatment of a viral infection in a subject, the method comprising the step of administering to the subject an effective amount of at least one compound of claim 1.

18. The method of claim 17, wherein the viral infection is HIV.

19. The method of claim 17, wherein the subject has been diagnosed with a need for treatment of the viral infection prior to the administering step.

20. The method of claim 17, further comprising the step of identifying a subject in need of treatment of the viral infection.
